SEQUENCE LISTING (13 sequences)

SEQ ID NO: 1 - DNA, 137 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 2 - DNA, 123 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 3 - DNA, 110 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 4 - DNA, 102 bp, Artificial Sequence
  Description of Artificial Sequence Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 5 - DNA, 88 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtgttttttt

SEQ ID NO: 6 - DNA, 76 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 7 - DNA, 20 bp, Artificial Sequence
  Description of Artificial Sequence Synthetic oligonucleotide
gtctacctgaccaatcgatg

SEQ ID NO: 8 - DNA, 20 bp, Artificial Sequence
  Description of Artificial Sequence Synthetic oligonucleotide
agcacagtagaactaagggt

SEQ ID NO: 9 - DNA, 20 bp, Artificial Sequence
  Description of Artificial Sequence Synthetic oligonucleotide
aattatggcaggttgttacg

SEQ ID NO: 10 - DNA, 20 bp, Artificial Sequence
  Description of Artificial Sequence Synthetic oligonucleotide
gcatgacgcctttccgcggc

SEQ ID NO: 11 - DNA, 20 bp, Artificial Sequence
  Description of Artificial Sequence Synthetic oligonucleotide
ggtggcggccgtttttcggg

SEQ ID NO: 12 - DNA, 20 bp, Artificial Sequence
  Description of Artificial Sequence Synthetic oligonucleotide
cggtggcggccgtttttcgg

SEQ ID NO: 13 - DNA, 3978 bp, Homo sapiens
agcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctcta

(19) United States
(12) Patent Application Publication
(10) Pub. No.: US 2024/0150839 A1
(43) Pub. Date: May 9, 2024

(54) METHODS FOR PREDICTING RESPONSIVENESS OF PROSTATE CANCER PATIENTS TO PARP INHIBITORS

(71) Applicant: Memorial Sloan Kettering Cancer Center, New York, NY (US)

(72) Inventors: Goutam CHAKRABORTY, New York, NY (US); Philip KANTOFF, New York, NY (US)

(21) Appl. No.: 17/773,288

(22) PCT Filed: Oct. 29, 2020
(86) PCT No.: PCT/US2020/058003

§ 371 Date: Apr. 29, 2022

Related U.S. Application Data
(60) Provisional application No. 62/928,286, filed on Oct. 30, 2019.

(51) Int. Cl. C12Q 1/6886; A61K 45/00

(52) U.S. Cl. CPC C12Q 1/6886; A61K 45/00; C12Q 2600/106; C12Q 2600/156

(57) ABSTRACT

The present disclosure provides methods for determining whether a patient diagnosed with or at risk for metastatic castration-resistant prostate cancer will benefit from or is predicted to be responsive to treatment with a PARP inhibitor. These methods are based on detecting a co-deletion in BRCA2 and RB1 in a biological sample obtained from a prostate cancer patient. Kits for use in practicing the methods are also provided.

### CROSS-REFERENCE TO RELATED APPLICATIONS

This application is a 371 U.S. national phase application of PCT/US2020/058003, filed Oct. 29, 2020, which claims the benefit of and priority to U.S. Provisional Patent Application No. 62/928,286, filed Oct. 30, 2019, the entire contents of which are incorporated herein by reference.

### STATEMENT OF GOVERNMENT INTEREST

This invention was made with government support under CA008748, awarded by the National Institutes of Health/National Cancer Institute, and CA228696-02, awarded by the National Cancer Institute. The government has certain rights in the invention.

### TECHNICAL FIELD

The present technology relates generally to methods for determining whether a patient diagnosed with or at risk for metastatic castration-resistant prostate cancer will benefit from or is predicted to be responsive to treatment with a PARP inhibitor. These methods are based on detecting a co-deletion in BRCA2 and RB1 in a biological sample obtained from a prostate cancer patient. Kits for use in practicing the methods are also provided.

### SEQUENCE LISTING

The instant application contains a Sequence Listing which has been submitted electronically in ASCII format and is hereby incorporated by reference in its entirety. Said ASCII copy, created on Dec. 7, 2020, is named 115872-2014_SL.txt and is 9,580 bytes in size.

### BACKGROUND

The following description of the background of the present technology is provided simply as an aid in understanding the present technology and is not admitted to describe or constitute prior art to the present technology.

Approximately 11.6 percent of men will be diagnosed with prostate cancer at some point during their lifetime. Pathologic variants of DNA damage response (DDR) genes are prevalent in a subset of men with metastatic castration-resistant prostate cancer (mCRPC). DDR is an essential defense and cell survival mechanism. Inherited (germline) or somatic genetic abnormalities of DDR pathway components, primarily insertions or deleterious mutations resulting in protein truncations, occur in 20%-25% of men with mCRPC. Recent observations have shown that alterations of BRCA2 are more prevalent than previously appreciated in men with prostate cancer and more frequent than alterations in any other DDR gene (Mandelker D et al., JAMA 318(9):825-35 (2017)). In one study, BRCA2 alterations were seen in 13.3% of men with metastatic prostate cancer, while another found germline BRCA2 mutations in 5.3% of men with advanced prostate cancer (Pritchard C C et al., N Engl J Med. 375(5):443-53 (2016), Robinson D et al., Cell 162(2):454 (2015)). Importantly, in a cohort of 1,302 men with localized and locally advanced prostate cancer, the 67 patients with BRCA2 germline mutations experienced more rapid progression to mCRPC, with 5-year metastasis-free survival rates of approximately 50%-60%, suggesting a more aggressive phenotype (Castro E et al., Eur Urol. 68(2):186-93 (2015)). Deep sequencing of cell-free DNA (cfDNA) from 202 patients with mCRPC treated with abiraterone acetate or enzalutamide after development of CRPC revealed that defects in BRCA2 and ATM were strongly associated with poor clinical outcomes and resistance to these second-generation antiandrogens, independent of other prognostic factors (Annala M et al., Cancer Discov. 8(4):444-57 (2018)). The mechanisms by which loss of BRCA2 might promote aggressive prostate cancer and confer resistance to androgen deprivation therapy (ADT) and androgen signaling pathway inhibitors are not understood.

### SUMMARY OF THE PRESENT TECHNOLOGY

In one aspect, the present disclosure provides a method for selecting a prostate cancer patient for treatment with a PARP inhibitor comprising: (a) detecting a co-deletion in BRCA2 and RB1 in a biological sample obtained from a prostate cancer patient; and (b) administering a PARP inhibitor to the prostate cancer patient, optionally wherein the co-deletion comprises a frameshift mutation or a nonsense mutation in each of BRCA2 and RB1. In some embodiments, the co-deletion results in the production of non-functional BRCA2 and RB1 polypeptides. The co-deletion in BRCA2 and RB1 may be homozygous or heterozygous. Examples of PARP inhibitors include, but are not limited to, olaparib, rucaparib, niraparib, talazoparib, veliparib, an inhibitory nucleic acid targeting PARP, and an anti-PARP neutralizing antibody. The inhibitory nucleic acid targeting PARP may be a shRNA, a siRNA, a sgRNA, a ribozyme, or an anti-sense oligonucleotide.

Additionally or alternatively, in some embodiments, the patient has not previously received an anti-cancer therapy. Examples of anti-cancer therapy include chemotherapy, radiation therapy, surgery or any combination thereof. In certain embodiments, the prostate cancer patient is diagnosed with or at risk for metastatic castration-resistant prostate cancer. The prostate cancer may be castration-resistant prostate cancer or primary (localized) prostate cancer. Additionally or alternatively, in some embodiments, the patient harbors a mutation in TP53 and/or ATM.

In any and all embodiments of the methods disclosed herein, the co-deletion in BRCA2 and RB1 is detected via polymerase chain reaction (PCR), reverse transcriptase polymerase chain reaction (RT-PCR), next-generation sequencing, Northern blotting, Southern blotting, microarray, dot or slot blots, fluorescent in situ hybridization (FISH), electrophoresis, chromatography, or mass spectroscopy. In certain embodiments, the biological sample is blood, plasma, serum, or a prostate tissue sample.

In another aspect, the present disclosure provides a method for treating or preventing metastatic castration-resistant prostate cancer in a patient in need thereof comprising administering to the patient an effective amount of a PARP inhibitor, wherein the patient harbors a co-deletion in BRCA2 and RB1, and wherein the co-deletion comprises a frameshift mutation or a nonsense mutation in each of BRCA2 and RB1. In some embodiments, the co-deletion results in the production of non-functional BRCA2 and RB1 polypeptides. The co-deletion in BRCA2 and RB1 may be homozygous or heterozygous. Examples of PARP inhibitors include, but are not limited to, olaparib, rucaparib, niraparib, talazoparib, veliparib, an inhibitory nucleic acid targeting PARP, and an anti-PARP neutralizing antibody. The inhibitory nucleic acid targeting PARP may be a shRNA, a siRNA, a sgRNA, or an anti-sense oligonucleotide.

Additionally or alternatively, in some embodiments, the patient has not previously received an anti-cancer therapy. Examples of anti-cancer therapy include chemotherapy, radiation therapy, surgery or any combination thereof. In certain embodiments, the prostate cancer patient is diagnosed with or at risk for metastatic castration-resistant prostate cancer. The prostate cancer may be castration-resistant prostate cancer or primary (localized) prostate cancer. Additionally or alternatively, in some embodiments, the patient harbors a mutation in TP53 and/or ATM.

In any and all embodiments of the methods disclosed herein, the co-deletion in BRCA2 and RB1 is detected via polymerase chain reaction (PCR), reverse transcriptase polymerase chain reaction (RT-PCR), next-generation sequencing, Northern blotting, Southern blotting, microarray, dot or slot blots, fluorescent in situ hybridization (FISH), electrophoresis, chromatography, or mass spectroscopy.

Also disclosed herein are kits for selecting a prostate cancer patient for treatment with a PARP inhibitor disclosed herein. The kits comprise reagents for detecting a co-deletion in BRCA2 and RB1 in a biological sample obtained from the patient. In some embodiments, the reagents for detecting a co-deletion in BRCA2 and RB1 include primers or probes that are complementary to a portion of the BRCA2 gene, along with primers or probes that are complementary to a portion of the RB1 gene. Additionally or alternatively, in some embodiments, the primers or probes comprise one or more detectable labels (e.g., fluorophores).

### BRIEF DESCRIPTION OF THE DRAWINGS

FIGS. 1A-1G demonstrate that the BRCA2 loss induces castration resistance in prostate cancer cells. FIG. 1A shows western blots of protein in LNCaP cells transduced with three different guide RNAs (gRNAs) targeting BRCA2 (CRISPR-BRCA2). Cells infected with scrambled (scr) gRNA were used as a negative control. Cas9 and RHoGDI served as loading controls. FIG. 1B shows the immunofluorescence study of phospho-gamma H2AX (pγH2AX) and DNA-PKcs (S2056) in BRCA2 CRISPR-edited LNCaP cells. Nuclei were stained with DAPI (blue). FIG. 1C shows bar graphs of pγH2AX and DNA-PKcs (S2056) positive foci counted in high power field. P-values were determined by Student's t-test. ***P<0.001. FIGS. 1D-1E shows a bar graph (FIG. 1D) and growth curve (FIG. 1E) of the proliferation of LNCaP BRCA2 CRISPR-edited and non-targeting control gRNA (scr) infected cells in charcoal-stripped medium (CSS) or complete medium supplemented with enzalutamide (ENZ; indicated concentration) for 7 days. Equivalent volume of DMSO was used as placebo treatment. Cell growth was measured by BRDU incorporation assay (see Example 1) (±SD); P-values were determined by Student's t-test.***P<0.001. FIG. 1F shows the analysis of BRCA2 mRNA by qPCR. Parental LAPC4 cells were transiently transfected with BRCA2-specific SMARTpool siRNA for 96 hours. Total RNA was isolated, and BRCA2 mRNA was analyzed by qPCR. Scrambled SMARTpool siRNA-transfected cells were used as control (top). BRCA2- or scrambled SMARTpool siRNA-transfected LAPC4 cells were cultured in charcoal-stripped medium (CSS) or complete medium supplemented with enzalutamide (ENZ; 20 μM) for 72 hours after transfection (bottom). Equivalent volume of DMSO was used as placebo treatment. Cell growth was measured by MTT assay; SD, p-values were determined by Student's t-test. FIG. 1G shows the effect of BRCA2 on 3D organoid growth. Control and CRISPR-edited LNCaP cells (10³ cells/well) were mixed with Matrigel, and 3D cell cultures (organoids) were grown for 7 days in androgen-depleted, growth factor-enriched media. The photographs show the picture of the 24-well plate at day 7 (top left) and the 40× magnification images of representative 3D organoids (bottom left). The graph (right) shows the number of 3D organoids (>100 μM diameter, ±SD); each point represents the number of organoids grown from 10³ cells in each individual well, p-value was determined by Student's t-test.

FIGS. 2A-2H demonstrate that co-loss of BRCA2 and RB1 induces invasive phenotype in LNCaP cells. FIG. 2A shows western blots of indicated protein levels in LNCaP-BRCA2 CRISPR-edited (CRISPR gRNA 2) and non-targeting gRNA infected control (Scr-CRISPR) cells infected with lentiviral RB1 short hairpin RNA (shRNA). Scr-CRISPR and BRCA2-CRISPR2 cells were also transfected with non-targeting shRNA (scr-Sh) for control of shRNA. RHoGDI served as the loading control. FIG. 2B shows the cell growth of indicated cells treated with 3 μM palbociclib (CDK4/6 inhibitor) for 3 days. Equivalent volume of DMSO was used as placebo treatment. Cell growth was measured by MTT assay; SD, p-values were determined by Student's t-test. FIG. 2C (Top row) shows the phase contrast bright field micrograph (200× magnification) of the morphology of LNCaP cells after infection with indicated CRISPR/shRNA in stable lentiviral vector. FIG. 2C (2^{nd} and 3^{rd} rows) show the immunofluorescence (400× magnification) of F-actin filament stained with phalloidin in indicated CRISPR/shRNA-infected LNCaP cells. Nuclei were stained with DAPI (blue). Note that LNCaP-BRCA2-RB1 cells exhibit cytoskeleton rearrangement compared to scrambled control LNCaP cells. FIG. 2C (4^{th} row) shows the micrographs (in 40× magnification) of 24-hour wound migration of indicated cells (see Example 1). FIG. 2C (bottom row) shows Matrigel invasion. 5×10³ indicated cells were plated on the top of Boyden chamber (see Example 1) in serum-free media; 10% serum in the bottom chamber was used as chemo-attractant. After 48 hours, cells in the lower side of the chamber were fixed, stained, and photographed (100× magnification). FIG. 2D shows the immunofluorescence images showing phospho-gamma H2AX (pγH2AX) and DNA-PKcs (S2056) in indicated LNCaP cells. Nuclei were stained with DAPI (blue). FIG. 2E shows the viability of the indicated cells treated with PARP inhibitors (olaparib 3 μM, talazoparib 0.005 μM) for indicated days. The graphs show cell growth measured by MTT assay (±SD); P-values determined by Student's t-test. FIG. 2F shows the RNA sequencing heat map generated by RNA sequencing followed by hierarchical clustering of the genes altered in LNCaP cells stably infected with indicated CRISPR/shRNA (false discovery rate [FDR]±0.1). RNA sequencing was analyzed by Partek. FIG. 2G (top panel) shows the volcano plot showing the genes altered in LNCaP cells stably co-infected with BRCA2 CRISPR and RB1 shRNA compared to scrambled gRNA- scrambled shRNA (scr) infected LNCaP cells.

FIG. 2G (bottom panel) shows the bar graph representing the disease-specific pathway analysis of the genes unregulated in BRCA2-RB1 knockout/knockdown LNCaP cells. Pathway analyses were performed using ToppGene. FIG. 2H shows the BRCA2-RB1 signature score (see Example 1) generated from the 10 most upregulated (top panel) or downregulated (bottom panel) genes in LNCaP-BRCA2-RB1 cells compared to control LNCaP cells from the RNA sequencing (FIG. 2F) and converted into an mRNA score using ssGSEA. Clinical significance of BRCA2-RB1 score determined by biochemical recurrence-free survival in Taylor primary prostate cancer cohort (n=131). Log-rank test was used to compare groups.

FIGS. 3A-3J demonstrate that the induction of EMT phenotype resulted in co-loss of BRCA2 and RB1 phenotype in LNCaP cells. FIG. 3A shows the western blots showing indicated protein levels in LNCaP-BRCA2 CRISPR-edited (CRISPR gRNA 2) and non-targeting gRNA infected control (Scr-CRISPR) cells infected with lentiviral RB1 short hairpin RNA (shRNA). Scr-CRISPR and BRCA2-CRISPR2 cells were also transfected with non-targeting shRNA (scr-Sh) for control of shRNA. GAPDH served as the loading control. FIG. 3B shows the immunofluorescence (400× magnification) of E-cadherin, vimentin and β-catenin on indicated CRISPR/shRNA knockdown and scrambled CRISPR control LNCaP cells. Nuclei were stained with DAPI (blue). Note that LNCaP-BRCA2-RB1 cells exhibited significant loss of cell surface E-cadherin and β-catenin but exhibited gain of vimentin compared to scrambled CRISPR control LNCaP cells. FIG. 3C shows the levels of indicated protein in BRCA2 and/or RB1 transiently overexpressed in PC3M cells as assayed by western blots. Control cells were transfected with empty vector. Western blot shows expression of indicated proteins. GAPDH served as the loading control. FIG. 3D shows the western blots showing BRCA2 and RB1 levels in RWPE1-BRCA2 CRISPR-edited (CRISPR gRNA 2) and non-targeting gRNA infected control cells. LNCaP cells were used as control. GAPDH served as the loading control. Note that RWPE1 cells exhibit significantly depleted RB1 protein compare to LNCaP cells. FIG. 3E (top panel) shows the phase contrast bright field micrograph (200× magnification) showing the morphology of RWPE1 cells after infection with BRCA2 CRISPR. FIG. 3E (middle panel) shows the immunofluorescence (400× magnification) of F-actin filament stained with phalloidin in indicated BRCA2 CRISPR-infected RWPE1 cells. Nuclei were stained with DAPI (blue). Note that RWPE1-BRCA2 cells exhibit cytoskeleton rearrangement compared to control RWPE1 cells. FIG. 3E (bottom panel) shows the micrographs (in 40× magnification) of 24-hour wound migration of indicated cells (see Example 1). FIG. 3F shows the immunofluorescence (400× magnification) of E-cadherin, vimentin and β-catenin on BRCA2 CRISPR-infected RWPE1 and CRISPR control RWPE1 cells. Note that RWPE1-BRCA2 cells exhibit significant loss of cell surface E-cadherin and β-catenin but exhibit gain of vimentin compared to control RWPE1 cells. FIG. 3G shows the BRCA2 CRISPR-infected RWPE1 and CRISPR control RWPE1 cells were treated with 3 μM and 10 μM olaparib for 7 days. Equivalent volume of DMSO was used as placebo treatment. Cell growth was measured by MTT assay; SD, P-values determined by Student's t-test. FIG. 3H shows the bar graph showing the changes in selected EMT and stem cell markers after co-elimination of BRCA2 and RB1 in LNCaP cells as determined by qPCR, compared to scrambled control cells. LNCaP-BRCA2-RB1 or control cells were incubated in charcoal-stripped medium (CSS) for 24 hours followed by treatment with 1 nM R1881 for another 48 hours (in CSS). FIG. 3I shows the bar graph showing the changes (via qPCR) of SLUG and PRRX1 in treated and untreated cells. Expression of the indicated genes normalized with untreated control and GAPDH. FIG. 3J shows the invasion in SLUG-, SNAIL- and PRRX1- or SMARTpool siRNA-transfected LNCaP-BRCA2-RB1 cells. Scrambled-SMARTpool siRNA transfected LNCaP cells were used as control. 2.5×10³ indicated cells (72 hours after indicated siRNA transfection) were plated on the top of Boyden chamber in serum-free media; 10% serum in the bottom chamber was used as chemo-attractant. After 24 hours, cells in the lower side of the chamber were fixed, stained, photographed in 100× magnification (top panel), and counted and represented in the form of the bar graph (bottom panel). P-values were determined by Student's t-test.

FIGS. 4A-4J demonstrate that concomitant deletion of BRCA2 and RB1 represents an aggressive variant of prostate cancer. FIG. 4A shows the alteration frequency of various DNA damage response (DDR) components in the cohort from Armenia et al., Nat Genet 50:645-51 (2018); P-values calculated by Fisher's exact test. FIG. 4B shows the significance of BRCA2 alteration (either homozygous or heterozygous deletion) and disease/progression-free survival (5 years) in TCGA provisional cohort (primary prostate cancer). Kaplan-Meier curves were calculated for BRCA2 wild-type (wt) (diploid+chromosomal gain) and BRCA2 homozygous or heterozygous deletion; the log-rank test was used to compare groups and to determine the significance. FIG. 4C shows the association between BRCA2 protein expression (reverse-phase protein arrays [RPPA]) and genomic deletion in TCGA cohort; P-value (±SD) and P-trend determined by one-way ANOVA. FIG. 4D (top panel) shows the co-deletion (homozygous or heterozygous) of BRCA2 and RB1 in TCGA provisional cohort. Note that BRCA2 is frequently deleted with RB1. FIG. 4D (bottom panel) shows the significance of co-deletion of BRCA2 and RB1 as determined by disease/progression-free survival in primary prostate cancer patients in the TCGA provisional cohort. Kaplan-Meier curves for 60 months were defined for each group. Log-rank test was used to compare groups. FIG. 4E shows the higher rates of co-deletion of BRCA2 and RB1 and higher risk in primary tumors and advanced-stage disease. Gleason grade and metastatic status are shown by alteration status in the cohort from Armenia et al., Nat Genet 50:645-51 (2018); P-value calculated by Fisher's exact test (FIG. 19). FIG. 4F shows the fraction of genome alteration (FGA) in prostate cancer patients with BRCA2 and/or RB1 deletion analyzed from primary and metastatic cases in the prostate cancer cohort from Armenia et al., Nat Genet 50:645-51 (2018) (±SD); individual blue circles indicate individual patients. Due to the very low number of cases with BRCA2 deletion only, those patients are not shown on this graph. P-values determined by Student's t-test; P-trends determined by one-way ANOVA. FIG. 4G shows the copy number (CN) segment analysis of BRCA2-RB1 region of chromosome 13q in the cohort from Armenia et al., Nat Genet 50:645-51 (2018). Samples are divided into primary and metastatic prostate cancer. FIG. 4H shows the copy number (top panel) and mRNA expression (bottom panel) of the chromosome 13q genes in TCGA 2015 cohort. Genes located in the region between BRCA2 and RB1 and outside this region are marked in different colors. Median expression of mRNA indicated by red lines. FIG. 4I shows the comparison between mean mRNA expression of the 13q genes in prostate cancer patients. The transcriptomic analyzed data from TCGA pan-cancer prostate cohort. Parents harboring BRCA2-RB1 co-deletion indicated as yellow and unaltered indicated as blue. The genes were divided in 3 groups on the basis of their chromosomal position (upstream from BRCA2 [n=69], in the region between BRCA2 and RB1 [n=63], or downstream from the BRCA2-RB1 region [n=150]) (±SD); P-values determined by Student's t-test. Each point represents a single gene. FIG. 4J shows the heat map (hierarchical clustering) of the mRNA expression of 63 genes (BRCA2-RB1 region of chromosome 13q) in primary and mCRPC samples in Grasso cohort. The heat map is generated in Oncomine suite. Genes are ranked on the basis of P-value and fold changes.

FIGS. 5A-5G demonstrate the concomitant heterozygous co-deletion of BRCA2-RB1 in prostate cancer cell lines. FIG. 5A shows the FISH analysis of indicated human prostate cancer cell lines using 3-color probes. The bar graph shows the deletion of BRCA2 and/or RB1 per 100 cells. Normal peripheral blood cells and RWPE1 cells were used as controls. FIG. 5B shows the BRCA2 and RB1 status in various prostate cancer cell lines in the Cancer Cell Line Encyclopedia. FIG. 5C shows the micrographs of FISH analysis of indicated human prostate cancer cell lines using a 3-color probe (red: BRCA2; orange: RB1; green: 13q 12, internal control). FIG. 5D shows a graph representing the copies of BRCA2, RB1 and 13q LNCaP cells analyzed by the 3-color FISH. Each point represents a single cell. A total of 100 individual cells from each cell line were counted and represented graphically. FIG. 5E shows the BRCA2 and RB1 protein expression in various prostate cancer cell lines as analyzed by western blot. RHoGDI was used as loading control. FIG. 5F shows the expression of the androgen receptor, vimentin and E-cadherin in human prostate cancer cells analyzed by western blots. GAPDH was used as loading control. FIG. 5G shows the viability of LNCaP and LNCaP-Abl cells treated with a PARP inhibitor (PARPi) (rucaparib [500 nM] or talazoparib [5 nM]) or cisplatin (500 nM) for 4 days. DMSO was used as a control. The graph shows cell growth measured by MTT assay (±SD); P-values determined by Student's t-test (***P<0.001).

FIGS. 6A-6E demonstrate that the organoids derived from mCRPC patients represent an experimental model for BRCA2-RB1 co-deletion. FIG. 6A shows the FISH analysis of indicated mCRPC-derived organoids (MSK-PCa 1-3) and benign prostate organoids using 3-color probes (see Example 1). FIG. 6B shows a bar graph showing the deletion of BRCA2 and/or RB1 per 100 cells. Near-diploid benign prostate organoid is used as a control. FIG. 6C shows the copy number (CN) segment analysis of BRCA2-RB1 region of chromosome 13q in mCRPC organoids. FIG. 6D shows western blots showing indicated protein levels in human mCRPC organoids. GAPDH served as the loading control. FIG. 6E shows the cell growth of organoids treated with PARPi (olaparib and talazoparib) in indicated concentrations for 3 days. The graphs show cell growth measured by MTT assay (±SD); P-trends determined by 2-way ANOVA.

FIGS. 7A-7G demonstrate the effect of BRCA2 deletion in prostate cancer. FIG. 7A shows a western blot (top panel) and qPCR (bottom panel) showing BRCA2 protein and mRNA in LNCaP cells transduced with three different guide RNAs (gRNAs) targeting BRCA2 (CRISPR-BRCA2). Cells infected with scrambled (scr) gRNA were used as control. Equal amount of proteins was loaded in WedgeWell 6% gel and western blot was performed. ˜400 kDa BRCA2 band is indicated by arrow. GAPDH served as loading controls for western blot and qPCR. FIG. 7B shows an ethidium bromide stained agarose gel showing BRCA2 genotype in BRCA2-CRISPR-edited LNCaP cells. Genotypes are detected by PCR amplification followed by being treated with T7 endonuclease. Wt and mutant BRCA2 are indicated by red and green arrows, respectively. FIG. 7C shows the growth curve of LNCaP CRISPR-edited cells cultured in complete medium supplemented with indicated amount of various PARP inhibitors or cisplatin for 6 days. Equivalent volume of DMSO was used as placebo treatment. Cell growth was measured by MTT assay (see Example 1); SD, P-values determined by Student's t-test. *P<0.05, **P<0.01, ***P<0.001. FIGS. 7D-7E show the growth curve (FIG. 7D) and a bar graph (FIG. 7E) representing the cell growth of LNCaP BRCA2 CRISPR-edited and non-targeting control gRNA (scr) infected cells in complete medium supplemented with enzalutamide (ENZ; indicated concentration) for 7 days or charcoal-stripped medium (CSS) for 5 days, respectively. Equivalent volume of DMSO was used as placebo treatment. Cell growth was measured by crystal violet staining assay (see Example 1) (±SD); P-values determined by Student's t-test. FIG. 7F shows the growth curve representing the growth of LNCaP BRCA2 CRISPR-edited and non-targeting control gRNA (scr) infected cells in charcoal-stripped medium (CSS) or complete medium supplemented with enzalutamide (ENZ; 20 μM) for indicated days. Equivalent volume of DMSO was used as placebo treatment. Cell growth was measured by MTT assay (see Example 1) (±SD); P-values determined by Student's t-test. FIG. 7G (left panel) shows the expression of BRCA2 mRNA. Parental LNCaP cells were transiently transfected with BRCA2-specific SMARTpool siRNA for 96 hours. Total RNA was isolated, and BRCA2 mRNA was analyzed by qPCR. Scrambled SMARTpool siRNA-transfected cells were used as control. FIG. 7G (right panel) shows the cell growth of BRCA2- or scrambled SMARTpool siRNA-transfected LNCaP cells cultured in charcoal-stripped medium (CSS) or complete medium supplemented with enzalutamide (ENZ; 20 μM) for 96 hours post transfection. Equivalent volume of DMSO was used as placebo treatment. Cell growth was measured by MTT assay; SD, P-values determined by Student's t-test.

FIGS. 8A-8J demonstrate that co-loss of BRCA2 and RB1 induces invasive phenotype. FIG. 8A shows the qPCR analysis showing BRCA2 and RB1 mRNA expression in LNCaP-BRCA2 CRISPR-edited (CRISPR gRNA 2) and scrambled control cells infected with lentiviral RB1 shRNA. Scr-CRISPR and BRCA2-CRISPR2 cells also transfected with non-targeting shRNA (scr-Sh) for control of shRNA. mRNA expression normalized with internal control (GAPDH). FIG. 8B shows the western blots showing RB1 expression in LNCaP cells transduced with three different guide RNAs (gRNAs) targeting BRCA2 (CRISPR-BRCA2). Cells infected with scrambled (scr) gRNA were used as control. Cas9 and RHoGDI served as loading controls. FIG. 8C shows the western blot showing RB1 and BRCA2 expression in LNCaP cells transduced with two different guide RNAs (gRNAs) targeting RB1 (CRISPR-RB1). Cells infected with scrambled (scr) gRNA were used as control. Cas9 and RHoGDI served as loading controls. FIG. 8D shows the invasion assays. 5×10³ of indicated cells were plated on the top of Boyden chamber (see Example 1) in serum-free media; 10% serum in the bottom chamber was used as chemo-attractant. After 24 hours, cells in the lower side of the chamber were fixed and stained, cells were counted and represented in the form of the bar graph. P-values were determined by Student's t-test. *P<0.05, **P<0.01, ***P<0.001. FIG. 8E shows the bar graphs showing pγH2AX and DNA-PKcs (S2056) positive foci counted in high power field (reference to FIG. 2D). P-values determined by Student's t-test. FIG. 8F shows the western blot showing RB1 in 22RV1 cells transduced with three different guide RNAs (gRNAs) targeting RB1 (CRISPR-RB1). Cells infected with scrambled (scr) gRNA were used as control. Cas9 and RHoGDI served as loading controls. FIG. 8G shows the Matrigel invasion assay. 2.5×10³ of RB1 CRISPR-edited or scrambled CRISPR control 22RV1 cells were plated on the top of Boyden chamber (see Example 1) in serum-free media; 10% serum in the bottom chamber was used as chemo-attractant. After 72 hours, cells in the lower side of the chamber were fixed, stained and photographed. FIG. 8H shows the bar graph representing the pathways (gene ontology-biological pathways) that are positively enriched in BRCA2-RB1 knockout/knockdown LNCaP cells. Pathway analyses were performed using gene set enrichment analysis (GSEA). FIGS. 8I-8J show the GSEA utilizing previously published RB1 signatures. Mateo et al., N Engl J Med. 373(18):1697-708 (2015); Pritchard et al., N Engl J Med. 375(5):443-53 (2016). NES, normalized enrichment score.

FIGS. 9A-9F demonstrate that the induction of EMT phenotype resulted in co-loss of BRCA2 and RB1. FIG. 9A shows a bar graph representing the pathway analysis of the genes unregulated in BRCA2-RB1 knockout/knockdown LNCaP cells. Pathway analyses were performed using GSEA. FIG. 9B shows the qPCR analysis showing vimentin mRNA expression in LNCaP-BRCA2 CRISPR-edited (CRISPR gRNA 2) and scrambled control cells infected with lentiviral RB1 shRNA. mRNA expression normalized with internal control (GAPDH). FIG. 9C shows the Venn diagram showing the common pathways from the transcriptome of TCGA (BRCA2-RB1 co-deleted vs wild-type) and LNCaP (BRCA2-RB1 knockout/knockdown vs scrambled control cells). FIG. 9D shows the migration and invasion assay. 1×10³ BRCA2 and RB1 transiently co-overexpressed PC3M cells (72 hours post transfection) were plated on the top of a Boyden chamber or Matrigel invasion chamber in serum-free media; 10% serum in the bottom chamber was used as chemo-attractant. After 24 hours, cells in the lower side of the chambers were fixed, stained with crystal violet, and photographed in 100× magnification. FIG. 9E shows the relative mRNA expression of EMT and stem cell markers in LNCaP-BRCA2-RB1 cells. FIG. 9F shows the Venn diagram shows the common hallmark pathways that are in lethal compared to indolent prostate cancer (Setlur prostate cancer; Swedish watchful waiting cohort) and LNCaP (BRCA2-RB1 knockout/knockdown vs control cells).

FIGS. 10A-10J demonstrate that concomitant deletion of BRCA2 and RB1 represents an aggressive variant of prostate cancer. FIG. 10A shows the pan-cancer analysis of BRCA2 alteration. Samples are customized on the basis of alteration frequency (≥5% cases of BRCA2 alteration) and total number of cases in each group (≥50 cases in each group). FIG. 10B shows the in-depth analysis of BRCA2 alterations (homozygous or heterozygous deletions and mutations) in multiple publicly available prostate cancer cohorts using cBioPortal. Cohorts are divided into primary (n=925) and metastatic castration-resistant (n=444) groups. FIG. 10C shows the graph representing the BRCA2 mRNA expression for wt and BRCA2-deleted (heterozygous or homozygous) patients in TCGA provisional cohort; P-trend determined by one-way ANOVA. Individual blue circles indicate individual patients. FIG. 10D shows the Kaplan-Meier curves. Patients from TCGA provisional cohort were divided into 3 groups on the basis of BRCA2 protein expression (reverse phase protein array [RPPA]; ≥+1; +1 to −1; ≤−1), and Kaplan-Meier curves described disease/progression-free survival (5 years) in each group. Log-rank test was performed to examine significance. FIG. 10E (top panel) shows the alteration status of BRCA2 and RB1 in MSK-IMPACT prostate cancer cohort. Co-occurrence of indicated genes, as presented by cBioPortal. FIG. 10E (bottom panel) shows the pie charts showing the percentage of BRCA2 alteration (mutation and homozygous deletion) and co-occurrence with RB1 homozygous deletion in primary prostate cancer and mCRPC in MSK-IMPACT prostate cancer cohort. FIG. 10F shows the concordance of BRCA2 and RB1 copy number in TCGA (primary prostate cancer) and Kumar (mCRPC) cohorts. Individual blue circles indicate individual patients. FIG. 10G shows the graph representing the RB1 mRNA expression in wt and RB1 deleted (homozygous and heterozygous) patients in TCGA and Kumar cohorts; SD, P-trends determined by one-way ANOVA. Individual blue circles indicate individual patients. FIG. 10H shows the concomitant deletion of BRCA2 and RB1; significance was determined by disease/progression-free survival in Taylor et al. primary prostate cancer cohort (n=131). Kaplan-Meier curves for indicated months were defined for each group. Log-rank test was used to calculate P-value. FIG. 10I shows a volcano plot showing the genes altered in Gleason 6 patients (BRCA2-RB1 co-deleted vs wt) in TCGA cohort. Individual circles indicate individual genes. Heat map showed the expression of genes that are upregulated in BRCA2-RB1 co-deleted prostate cancer patients compared to wt prostate cancer (in TCGA cohort) analyzed in primary and mCRPC samples in Taylor cohort. The heat map was generated in Oncomine Suite. Genes are ranked on the basis of P-value and fold changes. FIG. 10J shows the heat map (hierarchical clustering) of the mRNA expression of 63 genes (BRCA2-RB1 region of chromosome 13q) in primary and mCRPC samples in Taylor cohort. The heat map is generated in Oncomine Suite. Genes are ranked on the basis of P-value and fold changes.

FIGS. 11A-11D demonstrate the concomitant heterozygous co-deletion of BRCA2-RB1 in prostate cancer cell lines. FIG. 11A shows the micrographs of FISH analysis of indicated human prostate cancer cell lines using a 3-color probe (see Example 1). The ploidy of each cell line is indicated. FIG. 11B shows the BRCA2 and RB1 mRNA expression in various prostate cancer cell lines was analyzed by qPCR. FIG. 11C shows the profile of 12 major DDR genes in prostate cancer cell lines in The Cancer Cell Line Encyclopedia. FIG. 11D shows the graphs representing the growth of 22RV1 (BRCA2-mutated and RB1-wt; near diploid) and PC3M (BRCA2-RB1 co-deleted; triploid) cultured in or complete medium supplemented with olaparib (2.5 μM), veliparib (5 μM), niraparib (1 μM), rucaparib (500 nM), talazoparib (5 nM), and cisplatin (500 nM) for 6 days. Equivalent volume of DMSO was used as placebo treatment. Cell growth was measured by MTT assay (see Example 1); SD, each point indicates the MTT count of each well of 96-well plate, P-values determined by Student's t-test. **P<0.01, ****P<0.0001. Note that talazoparib (5 nM) exhibited more growth inhibition in PC3M cells compared to olaparib (2.5 μM), P<0.0001.

FIGS. 12A-12D demonstrate that organoids derived from human mCRPC patients harbor co-heterozygous deletion of BRCA2 and RB1. FIG. 12A shows a graph representing the copies of BRCA2, RB1 and 13q in human mCRPC organoids analyzed by the 3-color FISH. Each point represents a single cell. A total of 100 individual cells from each cell line were counted and represented graphically. FIG. 12B shows the mutation profile of 12 major DDR genes in mCRPC organoids analyzed in cBioPortal. FIG. 12C shows the SLUG, SNAIL and PRRX1 mRNA in the organoids analyzed by qPCR. FIG. 12D shows the concomitant deletion of BRCA2 and RB1; significance was determined by overall survival in TCGA pan-cancer cohort (excluding the prostate cancer cases; n=10,830). Kaplan-Meier curves for indicated months were defined for each group. Log-rank test was used to calculate P-value.

FIG. 13 shows the list of antibodies and reagents used herein.

FIG. 14 shows the list of top 10 upregulated and downregulated genes BRCA2-RB1 Vs SCR obtained from RNA sequencing of LNCaP cells infected with BRCA2 CRISPR and/or RB1 shRNA in a stable lentiviral vector.

FIGS. 15A-15B show Toppgene (FIG. 15A) and GSEA (FIG. 15B) pathway analysis of the genes upregulated upon co-deletion of BRCA and RB1 in LNCaP cells.

FIG. 16 shows the hallmark pathway analysis of the genes upregulated upon co-deletion of BRCA and RB1 in LNCaP cells.

FIG. 17 shows the hallmark pathway analysis in TCGA provisional cohort (BRCA2-RB1 deleted vs unaltered patients).

FIG. 18 shows the hallmark pathway analysis in Setlur prostate cancer cohort (5-year lethal vs indolent patients).

FIG. 19 shows the analysis of BRCA2 and RB1 co-deletion in Armenia et al. cohort and calculated P-values between different groups.

FIG. 20 shows the changes of mRNA expression in BRCA2-RB1 co-deleted vs unaltered patients with Gleason 6 disease in TCGA provisional cohort (FDR 0.25). Changes of mRNA expression calculated in cBioPortal.

FIG. 21 shows the BRCA2-RB1 deletion status in matched prostate cancer (localized and metastatic) samples in Kumar et al. mCRPC cohort.

FIG. 22 shows the list of chromosome 13q genes. Genes located inside the BRCA2-RB1 region denoted as inside (without highlight), all the rest denoted as outside (highlighted in gray).

FIG. 23 shows the FISH of BRCA2 and RB1 in patient-derived human organoids.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3^{rd} edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

The present disclosure identifies a previously uncharacterized prostate cancer subset characterized by concomitant deletions (homozygous and heterozygous) of BRCA2 and RB1. Further, the cell line-based models of the present disclosure demonstrate that even single copy loss of both BRCA2 and RB1 is sufficient to induce an aggressive phenotype in prostate cancer.

Previous case studies reported mCRPC progression in a patient with germline BRCA2 mutation and a newly emerged RB1 single copy number loss following treatment with PARP inhibitor olaparib. Ma et al., BMC Med Genet. 19: 185 (2018). Previous papers have suggested that Retinoblastoma (RB1) tumor suppressor gene loss drives transformation of prostate adenocarcinoma (PADC) to neuroendocrine prostate cancer variants (NEPC) resistant to antiandrogen therapy (AAT) (Wadosky K et al., Molecular & Cellular Oncology 4(2):e1291397 (2017)), which may also be one of the mechanisms of PARP inhibitors resistance. As shown in the Examples described herein, PARP inhibition unexpectedly and significantly attenuated growth of prostate cancer cell lines and organoids derived from human mCRPC that harbor not only homozygous but also heterozygous co-deletion of BRCA2 and RB1. Accordingly, the present disclosure demonstrates that co-deletion of BRCA2 and RB1 in a subset of prostate cancer patients is an independent genomic driver of therapy-resistant aggressive prostate cancer rather than the consequence of exposure to therapy, and that co-loss of BRCA2 and RB1 may induce an epithelial-to-mesenchymal transition (EMT) mediated by induction of the transcription factors SLUG or SNAIL or transcriptional co-activator PRRX1. Thus, the methods disclosed herein permit the early recognition and intervention using PARP inhibitor-based therapy in prostate cancer cases identified as having a BRCA2-RB1 co-deletion.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5”-A-G-T-3′ is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, a “deletion” refers to a genetic aberration in which at least a part of a chromosome or a gene sequence is lost or missing. Deletion of a number of nucleotides that is not evenly divisible by three will lead to a frameshift mutation, causing all of the codons occurring after the deletion to be read incorrectly during translation, thereby producing a severely altered and potentially nonfunctional protein.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of prostate cancer, such as castration-resistant prostate cancer (e.g., mCRPC). As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al., 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample. As used herein, preventing prostate cancer such as castration-resistant prostate cancer (e.g., mCRPC), includes preventing or delaying the initiation of symptoms of prostate cancer such as castration-resistant prostate cancer (e.g., mCRPC). As used herein, prevention of prostate cancer such as castration-resistant prostate cancer (e.g., mCRPC) also includes preventing a recurrence of one or more signs or symptoms of prostate cancer such as castration-resistant prostate cancer (e.g., mCRPC).

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a methylated target nucleic acid. Probes are typically at least about 10, 15, 20, 25, 30, 35, 40, 50, 60, 75, 100 nucleotides or more in length.

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

As used herein, the terms “target sequence” and “target nucleic acid sequence” refer to a specific nucleic acid sequence to be detected, or quantified in the sample to be analyzed. Alternatively, the terms “target sequence” and “target nucleic acid sequence” refer to a specific nucleic acid sequence to be modulated (e.g., inhibited or downregulated).

The term “PARP inhibitor” as used herein refers to an agent that inhibits gene expression and/or biological activity of PARP. Examples of PARP biological activity include, but are not limited to, enzymatic activity, substrate binding activity, homo- or hetero-dimerization activity, and binding to a cellular structure. Examples of PARP inhibitors include, but are not limited to, olaparib, rucaparib, niraparib, talazoparib, veliparib, inhibitory nucleic acids targeting PARP (e.g., shRNAs, siRNAs or anti-sense oligonucleotides), and anti-PARP neutralizing antibodies.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### PARP

Poly (ADP-ribose) polymerase (PARP) is a family of proteins involved in a number of cellular processes such as DNA repair, genomic stability, and programmed cell death. DNA damage may be caused by normal cell actions, UV light, some anticancer drugs, and radiation. The main role of PARP, which is found in the nucleus, is to detect and initiate an immediate cellular response to metabolic, chemical, or radiation-induced single-strand DNA breaks (SSB) by signaling the enzymatic machinery involved in the SSB repair. Once PARP detects a SSB, it binds to the DNA, undergoes a structural change, and begins the synthesis of a polymeric adenosine diphosphate ribose (poly (ADP-ribose) or PAR) chain, which acts as a signal for the other DNA-repairing enzymes. Target enzymes include DNA ligase III (LigIII), DNA polymerase beta (polo), and scaffolding proteins such as X-ray cross-complementing gene 1 (XRCC1). Upon completion of the repair process, the PAR chains are degraded via Poly(ADP-ribose) glycohydrolase (PARG). NAD+ is required as a substrate for generating ADP-ribose monomers. It is believed that overactivation of PARP may deplete the stores of cellular NAD+ and induce progressive ATP depletion and necrotic cell death, since glucose oxidation is inhibited. PARP is inactivated by caspase-3 cleavage during programmed cell death.

### PARP Inhibitors

In one aspect, the present disclosure provides inhibitory nucleic acids (e.g., sgRNAs, antisense RNAs, ribozymes, or shRNAs) that inhibit PARP expression and/or activity. The mammalian nucleic acid sequences of PARP are known in the art (e.g., NCBI Gene ID: 142). The inhibitory nucleic acids of the present technology may comprise a nucleic acid molecule that is complementary to a portion of a PARP nucleic acid sequence. In some embodiments, the inhibitory nucleic acids (e.g., sgRNAs, antisense RNAs, ribozymes, or shRNAs) target at least one exon and/or intron of PARP. An exemplary nucleic acid sequence of Homo sapiens PARP1 is provided below:

| (SEQ ID NO: 13) | |
|---|---|
| 1 | agcaatctat cagggaacgg cggtggccgg tgcggcgtgt tcggtggcgg ctctggccgc |
| 61 | tcaggcgcct gcggctgggt gagcgcacgc gaggcggcga ggcggcagcg tgtttctagg |
| 121 | tcgtggcgtc gggcttccgg agctttggcg gcagctaggg gaggatggcg gagtcttcgg |
| 181 | ataagctcta tcgagtcgag tacgccaaga gcgggcgcgc ctcttgcaag aaatgcagcg |
| 241 | agagcatccc caaggactcg ctccggatgg ccatcatggt gcagtcgccc atgtttgatg |
| 301 | gaaaagtccc acactggtac cacttctcct gcttctggaa ggtgggccac tccatccggc |
| 361 | accctgacgt tgaggtggat gggttctctg agcttcggtg ggatgaccag cagaaagtca |
| 421 | agaagacagc ggaagctgga ggagtgacag gcaaaggcca ggatggaatt ggtagcaagg |
| 481 | cagagaagac tctgggtgac tttgcagcag agtatgccaa gtccaacaga agtacgtgca |
| 541 | aggggtgtat ggagaagata gaaaagggcc aggtgcgcct gtccaagaag atggtggacc |
| 601 | cggagaagcc acagctaggc atgattgacc gctggtacca tccaggctgc tttgtcaaga |
| 661 | acagggagga gctgggtttc cggcccgagt acagtgcgag tcagctcaag ggcttcagcc |
| 721 | tccttgctac agaggataaa gaagccctga agaagcagct cccaggagtc aagagtgaag |
| 781 | gaaagagaaa aggcgatgag gtggatggag tggatgaagt ggcgaagaag aaatctaaaa |
| 841 | aagaaaaaga caaggatagt aagcttgaaa aagccctaaa ggctcagaac gacctgatct |
| 901 | ggaacatcaa ggacgagcta aagaaagtgt gttcaactaa tgacctgaag gagctactca |
| 961 | tcttcaacaa gcagcaagtg ccttctgggg agtcggcgat cttggaccga gtagctgatg |
| 1021 | gcatggtgtt cggtgccctc cttccctgcg aggaatgctc gggtcagctg gtcttcaaga |
| 1081 | gcgatgccta ttactgcact ggggacgtca ctgcctggac caagtgtatg gtcaagacac |
| 1141 | agacacccaa ccggaaggag tgggtaaccc caaaggaatt ccgagaaatc tcttacctca |
| 1201 | agaaattgaa ggttaaaaaa caggaccgta tattcccccc agaaaccagc gcctccgtgg |
| 1261 | cggccacgcc tccgccctcc acagcctcgg ctcctgctgc tgtgaactcc tctgcttcag |
| 1321 | cagataagcc attatccaac atgaagatcc tgactctcgg gaagctgtcc cggaacaagg |
| 1381 | atgaagtgaa ggccatgatt gagaaactcg gggggaagtt gacggggacg gccaacaagg |
| 1441 | cttccctgtg catcagcacc aaaaaggagg tggaaaagat gaataagaag atggaggaag |
| 1501 | taaaggaagc caacatccga gttgtgtctg aggacttcct ccaggacgtc tccgcctcca |
| 1561 | ccaagagcct tcaggagttg ttcttagcgc acatcttgtc cccttggggg gcagaggtga |
| 1621 | aggcagagcc tgttgaagtt gtggccccaa gagggaagtc aggggctgcg ctctccaaaa |
| 1681 | aaagcaaggg ccaggtcaag gaggaaggta tcaacaaatc tgaaaagaga atgaaattaa |
| 1741 | ctcttaaagg aggagcagct gtggatcctg attctggact ggaacactct gcgcatgtcc |
| 1801 | tggagaaagg tgggaaggtc ttcagtgcca cccttggcct ggtggacatc gttaaaggaa |
| 1861 | ccaactccta ctacaagctg cagcttctgg aggacgacaa ggaaaacagg tattggatat |
| 1921 | tcaggtcctg gggccgtgtg ggtacggtga tcggtagcaa caaactggaa cagatgccgt |
| 1981 | ccaaggagga tgccattgag cacttcatga aattatatga agaaaaaacc gggaacgctt |
| 2041 | ggcactccaa aaatttcacg aagtatccca aaaagttcta ccccctggag attgactatg |
| 2101 | gccaggatga agaggcagtg aagaagctga cagtaaatcc tggcaccaag tccaagctcc |
| 2161 | ccaagccagt tcaggacctc atcaagatga tctttgatgt ggaaagtatg aagaaagcca |
| 2221 | tggtggagta tgagatcgac cttcagaaga tgcccttggg gaagctgagc aaaaggcaga |
| 2281 | tccaggccgc atactccatc ctcagtgagg tccagcaggc ggtgtctcag ggcagcagcg |
| 2341 | actctcagat cctggatctc tcaaatcgct tttacaccct gatcccccac gactttggga |
| 2401 | tgaagaagcc tccgctcctg aacaatgcag acagtgtgca ggccaaggtg gaaatgcttg |
| 2461 | acaacctgct ggacatcgag gtggcctaca gtctgctcag gggagggtct gatgatagca |
| 2521 | gcaaggatcc catcgatgtc aactatgaga agctcaaaac tgacattaag gtggttgaca |
| 2581 | gagattctga agaagccgag atcatcagga agtatgttaa gaacactcat gcaaccacac |
| 2641 | acaatgcgta tgacttggaa gtcatcgata tctttaagat agagcgtgaa ggcgaatgcc |
| 2701 | agcgttacaa gccctttaag cagcttcata accgaagatt gctgtggcac gggtccagga |
| 2761 | ccaccaactt tgctgggatc ctgtcccagg gtcttcggat agccccgcct gaagcgcccg |
| 2821 | tgacaggcta catgtttggt aaagggatct atttcgctga catggtctcc aagagtgcca |
| 2881 | actactgcca tacgtctcag ggagacccaa taggcttaat cctgttggga gaagttgccc |
| 2941 | ttggaaacat gtatgaactg aagcacgctt cacatatcag caagttaccc aagggcaagc |
| 3001 | acagtgtcaa aggtttgggc aaaactaccc ctgatccttc agctaacatt agtctggatg |
| 3061 | gtgtagacgt tcctcttggg accgggattt catctggtgt gaatgacacc tctctactat |
| 3121 | ataacgagta cattgtctat gatattgctc aggtaaatct gaagtatctg ctgaaactga |
| 3181 | aattcaattt taagacctcc ctgtggtaat tgggagaggt agccgagtca cacccggtgg |
| 3241 | ctctggtatg aattcacccg aagcgcttct gcaccaactc acctggccgc taagttgctg |
| 3301 | atgggtagta cctgtactaa accacctcag aaaggatttt acagaaacgt gttaaaggtt |
| 3361 | ttctctaact tctcaagtcc cttgttttgt gttgtgtctg tggggagggg ttgttttggg |
| 3421 | gttgtttttg ttttttcttg ccaggtagat aaaactgaca tagagaaaag gctggagaga |
| 3481 | gattctgttg catagactag tcctatggaa aaaaccaagc ttcgttagaa tgtctgcctt |
| 3541 | actggtttcc ccagggaagg aaaaatacac ttccaccctt ttttctaagt gttcgtcttt |
| 3601 | agttttgatt ttggaaagat gttaagcatt tatttttagt taaaaataaa aactaatttc |
| 3661 | atactattta gattttcttt tttatcttgc acttattgtc ccctttttag ttttttttgt |
| 3721 | ttgcctcttg tggtgagggg tgtgggaaga ccaaaggaag gaacgctaac aatttctcat |
| 3781 | acttagaaac aaaaagagct ttccttctcc aggaatactg aacatgggag ctcttgaaat |
| 3841 | atgtagtatt aaaagttgca tttgaaattc ttgactttct tatgggcact tttgtcttcc |
| 3901 | aaattaaaac tctaccacaa atatacttac ccaagggcta atagtaatac tcgattaaaa |
| 3961 | atgcagatgc cttctcta |

The present disclosure also provides an antisense nucleic acid comprising a nucleic acid sequence that is complementary to and specifically hybridizes with a portion of a PARP mRNA. The antisense nucleic acid may be antisense RNA, or antisense DNA. Antisense nucleic acids based on the known nucleic acid sequences of PARP can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire PARP coding strand, or to a portion thereof, e.g., all or part of the protein coding region (or open reading frame). In some embodiments, the antisense nucleic acid is an oligonucleotide which is complementary to only a portion of the coding region of PARP mRNA. In certain embodiments, an antisense nucleic acid molecule can be complementary to a noncoding region of the PARP coding strand. In some embodiments, the noncoding region refers to the 5′ and 3′ untranslated regions that flank the coding region and are not translated into amino acids. For example, the antisense oligonucleotide can be complementary to the region surrounding the translation start site of PARP. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual (β-units, the strands run parallel to each other (Gaultier et al., Nucleic Acids. Res. 15:6625-6641(1987)). The antisense nucleic acid molecule can also comprise a 2′-O-methylribonucleotide (Inoue et al., Nucleic Acids Res. 15:6131-6148 (1987)) or a chimeric RNA-DNA analogue (Inoue et al., FEBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of a PARP mRNA, thereby reducing or inhibiting PARP expression. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hybridize to an mRNA of a target gene. Following intracellular delivery, the siRNA or shRNA molecule associates with an RNA-induced silencing complex (RISC), which then binds and degrades a complementary target mRNA (such as PARP mRNA).

The present disclosure also provides a ribozyme comprising a nucleic acid sequence that is complementary to and specifically hybridizes with a portion of a PARP mRNA, thereby reducing or inhibiting PARP expression. Ribozymes are catalytic RNA molecules with ribonuclease activity which are capable of cleaving a complementary single-stranded nucleic acid, such as an mRNA. Thus, ribozymes (e.g., hammerhead ribozymes (described in Haselhoff and Gerlach, Nature 334:585-591 (1988))) can be used to catalytically cleave PARP transcripts, thereby inhibiting translation of PARP.

A ribozyme having specificity for a PARP-encoding nucleic acid can be designed based upon a PARP nucleic acid sequence. For example, a derivative of a Tetrahymena L-19 IVS RNA can be constructed in which the nucleotide sequence of the active site is complementary to the nucleotide sequence to be cleaved in a PARP-encoding mRNA. See, e.g., U.S. Pat. Nos. 4,987,071 and 5,116,742. Alternatively, PARP mRNA can be used to select a catalytic RNA having a specific ribonuclease activity from a pool of RNA molecules. See, e.g., Bartel and Szostak (1993) Science 261:1411-1418, incorporated herein by reference.

The present disclosure also provides a synthetic guide RNA (sgRNA) comprising a nucleic acid sequence that is complementary to and specifically hybridizes with a portion of a PARP nucleic acid sequence. Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of a PARP nucleic acid sequence, and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465.

In some embodiments, a synthetic guide RNA is a single RNA represented as comprising the following elements: 5′-X1-X2-Y-Z-3′ where X1 and X2 represent the crRNA segment, where X1 is the targeting sequence that binds to a portion of a PARP nucleic acid sequence, X2 is a stem sequence that hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of a PARP nucleic acid sequence, X2 is a stem sequence that hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator:

(a)
(SEQ ID NO: 1)
NNNNNNNNNNNNNNNNNNNNgtttttgtactctcaagatt
taGAAAtaaatcttgcagaagctacaaagataaggcttca
tgccgaaatcaacaccctgtcattttatggcagggtgttt
tcgttatttaaTTTTTT;
(b)
(SEQ ID NO: 2)
NNNNNNNNNNNNNNNNNNNNgtttttgtactctcaGAAAt
gcagaagctacaaagataaggcttcatgccgaaatcaaca
ccctgtcattttatggcagggtgttttcgttatttaaTTT
TTT;
(c)
(SEQ ID NO: 3)
NNNNNNNNNNNNNNNNNNNNgtttttgtactctcaGAAAt
gcagaagctacaaagataaggcttcatgccgaaatcaaca
ccctgtcattttatggcagggtgtTTTTTT;
(d)
(SEQ ID NO: 4)
NNNNNNNNNNNNNNNNNNNNgttttagagctaGAAAtagc
aagttaaaataaggctagtccgttatcaacttgaaaaagt
ggcaccgagtcggtgcTTTTTT;
(e)
(SEQ ID NO: 5)
NNNNNNNNNNNNNNNNNNNNgttttagagctaGAAATAGc
aagttaaaataaggctagtccgttatcaacttgaaaaagt
gTTTTTTT;
and
(f)
(SEQ ID NO: 6)
NNNNNNNNNNNNNNNNNNNNgttttagagctagAAATAGc
aagttaaaataaggctagtccgttatcaTTTTTTTT.

Selection of suitable oligonucleotides for use as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally, the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

In another aspect, the present disclosure provides pharmacological inhibitors of PARP including, but not limited to olaparib, rucaparib, niraparib, talazoparib, and veliparib. Anti-PARP neutralizing antibodies may also be employed in the methods disclosed herein.

### Pharmaceutical Compositions Including the PARP Inhibitors of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

Polynucleotides containing gene sequence alterations (e.g., deletions) may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

Nucleic Acid Amplification and/or Detection

Polynucleotides containing deletions in BRCA2 and RB1 can be detected by the use of nucleic acid amplification techniques that are well known in the art. The starting material may be genomic DNA, cDNA, RNA or mRNA. Nucleic acid amplification can be linear or exponential. Specific mutations (e.g., deletions) may be detected by the use of amplification methods with the aid of oligonucleotide primers or probes designed to interact with or hybridize to a particular target sequence in a specific manner, thus amplifying the target sequence.

Non-limiting examples of nucleic acid amplification techniques include polymerase chain reaction (PCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5@, Cy5®, Cy5.5@; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes can be designed so that they hybridize under stringent conditions to BRCA2 and/or RB1 target nucleic acid sequences in humans. In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, for example, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

NGS Platforms. Polynucleotides containing human-specific SNPs associated with cancer susceptibility can be detected using high throughput, massively parallel sequencing (a.k.a., next generation sequencing). In some embodiments, high throughput, massively parallel sequencing employs sequencing-by-synthesis with reversible dye terminators. In certain embodiments, sequencing is performed via sequencing-by-ligation. In other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Theranostic Methods of the Present Technology

In one aspect, the present disclosure provides a method for selecting a prostate cancer patient for treatment with a PARP inhibitor comprising: (a) detecting a co-deletion in BRCA2 and RB1 in a biological sample obtained from a prostate cancer patient; and (b) administering a PARP inhibitor to the prostate cancer patient. In some embodiments, the co-deletion comprises a frameshift mutation or a nonsense mutation in each of BRCA2 and RB1. In some embodiments, the co-deletion results in the production of non-functional BRCA2 and RB1 polypeptides. The co-deletion in BRCA2 and RB1 may be homozygous or heterozygous. Examples of PARP inhibitors include, but are not limited to, olaparib, rucaparib, niraparib, talazoparib, veliparib, an inhibitory nucleic acid targeting PARP, and an anti-PARP neutralizing antibody. The inhibitory nucleic acid targeting PARP may be a shRNA, a siRNA, a sgRNA, a ribozyme, or an anti-sense oligonucleotide. Additionally or alternatively, in some embodiments, the prostate cancer patient is human.

Additionally or alternatively, in some embodiments, the patient has not previously received an anti-cancer therapy. Examples of anti-cancer therapy include chemotherapy, radiation therapy, surgery or any combination thereof. In certain embodiments, the prostate cancer patient is diagnosed with or at risk for metastatic castration-resistant prostate cancer. The prostate cancer may be castration-resistant prostate cancer or primary (localized) prostate cancer. Additionally or alternatively, in some embodiments, the patient harbors a mutation in TP53 and/or ATM.

In any and all embodiments of the methods disclosed herein, the co-deletion in BRCA2 and RB1 is detected via polymerase chain reaction (PCR), reverse transcriptase polymerase chain reaction (RT-PCR), next-generation sequencing, Northern blotting, Southern blotting, microarray, dot or slot blots, fluorescent in situ hybridization (FISH), electrophoresis, chromatography, or mass spectroscopy. In certain embodiments, the biological sample is blood, plasma, serum, or a prostate tissue sample.

In another aspect, the present disclosure provides a method for treating or preventing metastatic castration-resistant prostate cancer in a patient in need thereof comprising administering to the patient an effective amount of a PARP inhibitor, wherein the patient harbors a co-deletion in BRCA2 and RB1. In some embodiments, the co-deletion comprises a frameshift mutation or a nonsense mutation in each of BRCA2 and RB1. In some embodiments, the co-deletion results in the production of non-functional BRCA2 and RB1 polypeptides. The co-deletion in BRCA2 and RB1 may be homozygous or heterozygous. Examples of PARP inhibitors include, but are not limited to, olaparib, rucaparib, niraparib, talazoparib, veliparib, an inhibitory nucleic acid targeting PARP, and an anti-PARP neutralizing antibody. The inhibitory nucleic acid targeting PARP may be a shRNA, a siRNA, a sgRNA, or an anti-sense oligonucleotide. Additionally or alternatively, in some embodiments, the prostate cancer patient is human.

Additionally or alternatively, in some embodiments, the patient has not previously received an anti-cancer therapy. Examples of anti-cancer therapy include chemotherapy, radiation therapy, surgery or any combination thereof. In certain embodiments, the prostate cancer patient is diagnosed with or at risk for metastatic castration-resistant prostate cancer. The prostate cancer may be castration-resistant prostate cancer or primary (localized) prostate cancer. Additionally or alternatively, in some embodiments, the patient harbors a mutation in TP53 and/or ATM.

In any and all embodiments of the methods disclosed herein, the co-deletion in BRCA2 and RB1 is detected via polymerase chain reaction (PCR), reverse transcriptase polymerase chain reaction (RT-PCR), next-generation sequencing, Northern blotting, Southern blotting, microarray, dot or slot blots, fluorescent in situ hybridization (FISH), electrophoresis, chromatography, or mass spectroscopy.

In therapeutic applications, compositions or medicaments comprising a PARP inhibitor disclosed herein are administered to a subject suspected of, or already suffering from such a disease or condition (such as a subject diagnosed with castration-resistant prostate cancer (e.g., mCRPC) and/or a subject diagnosed with prostate cancer), in an amount sufficient to cure, or at least partially arrest, the symptoms of the disease, including its complications and intermediate pathological phenotypes in development of the disease.

Subjects diagnosed with prostate cancer such as castration-resistant prostate cancer (e.g., mCRPC) can be identified by any or a combination of diagnostic or prognostic assays known in the art.

In some embodiments, subjects suffering from prostate cancer, such as castration-resistant prostate cancer (e.g., mCRPC), that are treated with the PARP inhibitor will show amelioration or elimination of one or more of the following symptoms: frequent urination, weak or interrupted urine flow or the need to strain to empty the bladder, the urge to urinate frequently at night, blood in the urine, blood in the seminal fluid, new onset of erectile dysfunction, pain or burning during urination, discomfort or pain when sitting, caused by an enlarged prostate.

In certain embodiments, subjects suffering from castration-resistant prostate cancer (e.g., mCRPC), and/or subjects suffering from prostate cancer that are treated with the PARP inhibitor will show reduced levels of EMT, metastasis or invasive phenotype and/or reduced PARP activity levels compared to untreated subjects suffering from castration-resistant prostate cancer (e.g., mCRPC)

In one aspect, the present technology provides a method for preventing or delaying the onset of prostate cancer, such as castration-resistant prostate cancer (e.g., mCRPC). Subjects at risk or susceptible to prostate cancer, or castration-resistant prostate cancer (e.g., mCRPC), include those that exhibit one or more mutations in BRCA2 and RB, increased levels of EMT, metastasis, or invasive phenotype. Such subjects can be identified by, e.g., any or a combination of diagnostic or prognostic assays known in the art.

In prophylactic applications, pharmaceutical compositions or medicaments comprising a PARP inhibitor disclosed herein are administered to a subject susceptible to, or otherwise at risk of prostate cancer or castration-resistant prostate cancer (e.g., mCRPC), in an amount sufficient to eliminate or reduce the risk, or delay the onset of the disease, including biochemical, histologic and/or behavioral symptoms of the disease, its complications and intermediate pathological phenotypes presenting during development of the disease. Administration of a prophylactic PARP inhibitor can occur prior to the manifestation of symptoms characteristic of the disease or disorder, such that the disease or disorder is prevented or, alternatively, delayed in its progression.

In some embodiments, treatment with the PARP inhibitor will prevent or delay the onset of one or more of the following symptoms: frequent urination, weak or interrupted urine flow or the need to strain to empty the bladder, the urge to urinate frequently at night, blood in the urine, blood in the seminal fluid, new onset of erectile dysfunction, pain or burning during urination, discomfort or pain when sitting, caused by an enlarged prostate.

For therapeutic and/or prophylactic applications, a composition comprising a PARP inhibitor disclosed herein, is administered to the subject. In some embodiments, the PARP inhibitor is administered one, two, three, four, or five times per day. In some embodiments, the PARP inhibitor is administered more than five times per day. Additionally or alternatively, in some embodiments, the PARP inhibitor is administered every day, every other day, every third day, every fourth day, every fifth day, or every sixth day. In some embodiments, the PARP inhibitor is administered weekly, bi-weekly, tri-weekly, or monthly. In some embodiments, the PARP inhibitor is administered for a period of one, two, three, four, or five weeks. In some embodiments, the PARP inhibitor is administered for six weeks or more. In some embodiments, the PARP inhibitor is administered for twelve weeks or more. In some embodiments, the PARP inhibitor is administered for a period of less than one year. In some embodiments, the PARP inhibitor is administered for a period of more than one year. In some embodiments, the PARP inhibitor is administered throughout the subject's life.

In some embodiments of the methods of the present technology, the PARP inhibitor is administered daily for 1 week or more. In some embodiments of the methods of the present technology, the PARP inhibitor is administered daily for 2 weeks or more. In some embodiments of the methods of the present technology, the PARP inhibitor is administered daily for 3 weeks or more. In some embodiments of the methods of the present technology, the PARP inhibitor is administered daily for 4 weeks or more. In some embodiments of the methods of the present technology, the PARP inhibitor is administered daily for 6 weeks or more. In some embodiments of the methods of the present technology, the PARP inhibitor is administered daily for 12 weeks or more. In some embodiments, the PARP inhibitor is administered daily throughout the subject's life.

### Modes of Administration and Effective Dosages

Any method known to those in the art for contacting a cell, organ or tissue with one or more PARP inhibitors disclosed herein may be employed. Suitable methods include in vitro, ex vivo, or in vivo methods. In vivo methods typically include the administration of one or more PARP inhibitors to a mammal, suitably a human. When used in vivo for therapy, the one or more PARP inhibitors described herein are administered to the subject in effective amounts (i.e., amounts that have desired therapeutic effect). The dose and dosage regimen will depend upon the degree of the disease state of the subject, the characteristics of the particular PARP inhibitor used, e.g., its therapeutic index, and the subject's history.

The effective amount may be determined during pre-clinical trials and clinical trials by methods familiar to physicians and clinicians. An effective amount of one or more PARP inhibitors useful in the methods may be administered to a mammal in need thereof by any of a number of well-known methods for administering pharmaceutical compounds. The PARP inhibitor may be administered systemically or locally.

The one or more PARP inhibitors described herein can be incorporated into pharmaceutical compositions for administration, singly or in combination, to a subject for the treatment or prevention of prostate cancer such as castration-resistant prostate cancer (e.g., mCRPC). Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more PARP inhibitors disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more PARP inhibitors disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more PARP inhibitor concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more PARP inhibitors may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, one or more of the PARP inhibitors disclosed herein may be combined with one or more additional therapies for the prevention or treatment of prostate cancer such as castration-resistant prostate cancer (e.g., mCRPC). Additional therapeutic agents include, but are not limited to, Abiraterone Acetate, Apalutamide, Bicalutamide, Cabazitaxel, Darolutamide, Degarelix, Docetaxel, Leuprolide Acetate, Enzalutamide, Flutamide, Goserelin Acetate, Mitoxantrone Hydrochloride, Nilutamide, Darolutamide, Sipuleucel-T, Radium 223 Dichloride, surgery, radiation, or a combination thereof.

In some embodiments, the one or more PARP inhibitors disclosed herein may be separately, sequentially or simultaneously administered with at least one additional therapeutic agent selected from the group consisting of alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, cytostatic alkaloids, cytotoxic antibiotics, antimetabolites, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent.

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits for selecting a prostate cancer patient for treatment with a PARP inhibitor disclosed herein. The kits comprise reagents for detecting a co-deletion in BRCA2 and RB1 in a biological sample obtained from the patient. In some embodiments, the reagents for detecting a co-deletion in BRCA2 and RB1 include primers or probes that are complementary to a portion of the BRCA2 gene, along with primers or probes that are complementary to a portion of the RB1 gene. Additionally or alternatively, in some embodiments, the primers or probes comprise one or more detectable labels (e.g., fluorophores). Optionally, the above described components of the kits of the present technology are packed in suitable containers and labeled for selecting a prostate cancer patient for treatment with a PARP inhibitor disclosed herein.

The kits are useful for selecting a prostate cancer patient for treatment with one or more PARP inhibitors disclosed herein based on the detection of a co-deletion in BRCA2 and RB1 in a biological sample, e.g., any body fluid including, but not limited to, e.g., serum, plasma, lymph, cystic fluid, urine, stool, cerebrospinal fluid, ascitic fluid or blood and including prostate tissue samples. The biological sample may be Formalin-Fixed Paraffin-Embedded (FFPE) tissue samples, fresh tissue samples or frozen tissue samples. For example, the kit can comprise primers or probes that are complementary to a portion of the BRCA2 gene, along with primers or probes that are complementary to a portion of the RB1 gene. One or more of the primers or probes may be labeled. The kit components, (e.g., reagents) can be packaged in a suitable container. The kit can further comprise instructions for using the kit to select a prostate cancer patient based on the detection of a co-deletion in BRCA2 and RB1.

The present disclosure also provides kits for the prevention and/or treatment of castration-resistant prostate cancer (e.g., mCRPC), comprising a) reagents for detecting a co-deletion in BRCA2 and RB1 in a biological sample; and b) one or more PARP inhibitors disclosed herein.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, and the like. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### Examples

The present technology is further illustrated by the following Examples, which should not be construed as limiting in any way. The examples herein are provided to illustrate advantages of the present technology and to further assist a person of ordinary skill in the art with preparing or using the compositions and methods of the present technology. The examples can include or incorporate any of the variations, aspects, or embodiments of the present technology described above.

### Example 1: Experimental Materials and Methods

Cell Culture. Human prostate cancer cells LNCaP, 22RV1, DU145, PC3, and VCaP were obtained from ATCC (Manassas, VA). LNCaP-C42 cells were obtained from VitroMed (Burlington, NC). The LNCaP-Abl cell line, E006AA-T cells, PC3M LAPC4 cell line were obtained. These cells were maintained in 10% FBS (LNCaP, LNCaP-C42, LAPC4, VCaP, 22RV1, DU145, PC3, PC3M, and E006AA) or 10% charcoal-stripped serum (LNCaP-Abl) supplemented with 2 mM of L-glutamine and 1×antibiotic/antimycotic (Gemini Bio-Products, Sacramento, CA) at 37° C. in 5% CO₂. Human prostate epithelial cell RWPE1 was obtained from ATCC and cultured in keratinocyte serum-free medium (Thermo Fisher Scientific, Waltham, MA) at 37° C. in 5% CO₂. Cells were authenticated by human short tandem repeat profiling at the MSK Integrated Genomics Operation Core. Patient-derived human prostate cancer organoids were cultured as described. Gao et al., Cell 159:176-87 (2014).

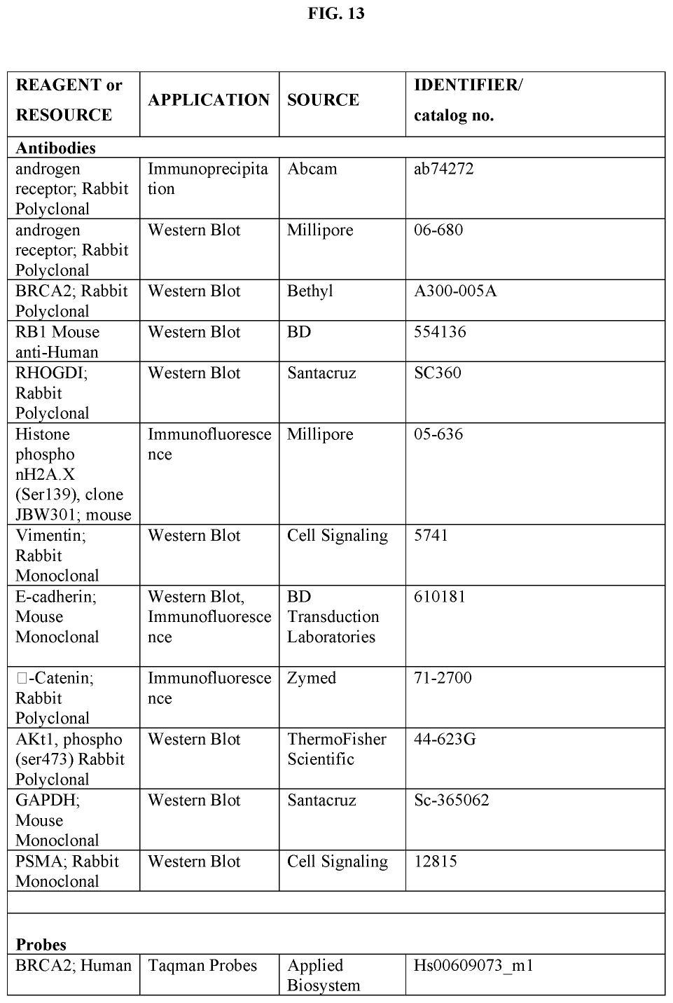

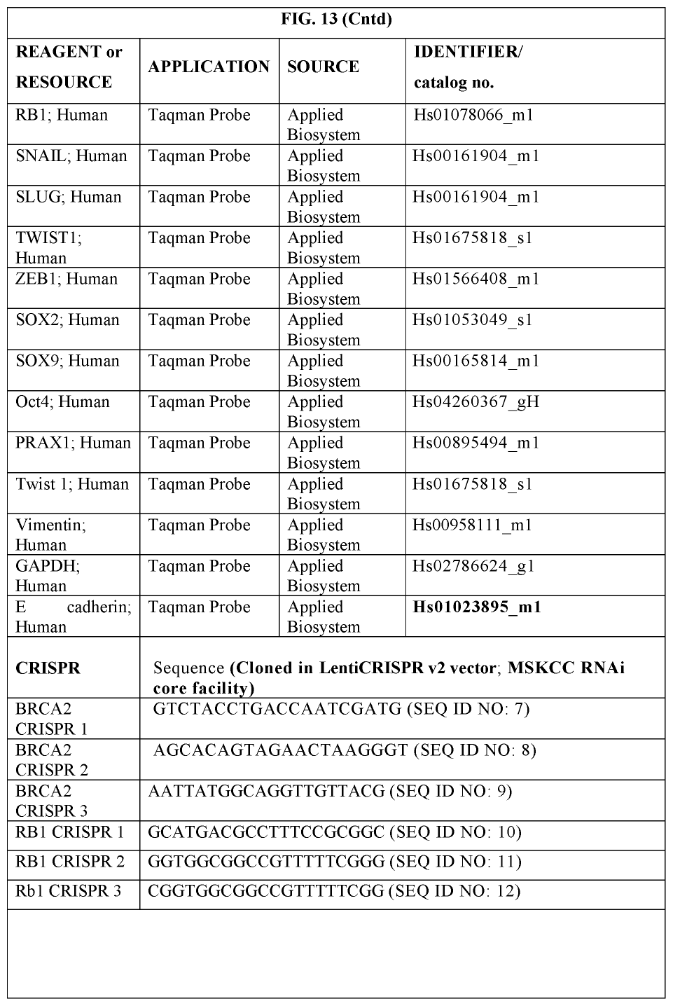

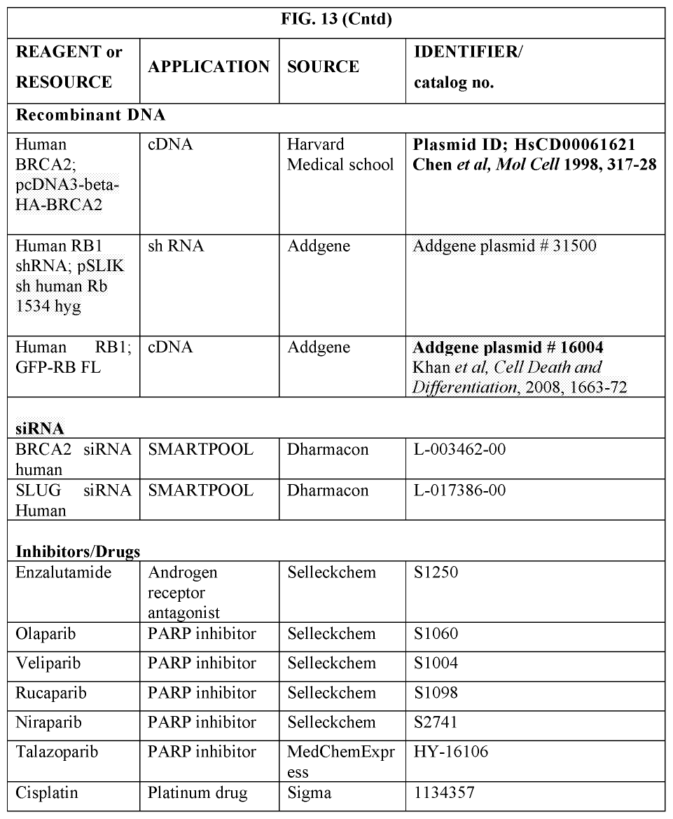

CRISPR, Gene Expression, and Gene Silencing. Lentiviral vectors encoding CRISPR or short hairpin RNA (shRNA) were generated as previously described (Komura et al., Proc Natl Acad Sci USA 113:6259-64 (2016)) and transfected to LNCaP cells using LentiBlast (OZ Biosciences, Marseille, France). Stable cells were generated using puromycin and/or hygromycin selection. Three separate guide RNAs (gRNA) were designed for human BRCA2 and human RB1 (FIG. 13) and cloned the gRNAs into a LentiCRISPRv2-puromycin or hygromycin backbone respectively; a third generation lentiviral backbone that constitutively expresses Cas9. Nontargeting scrambled gRNA (scr gRNA) was used as control. A similar strategy was used for generating 22RV1-RB1 cells and LNCaP-RB1 cells.

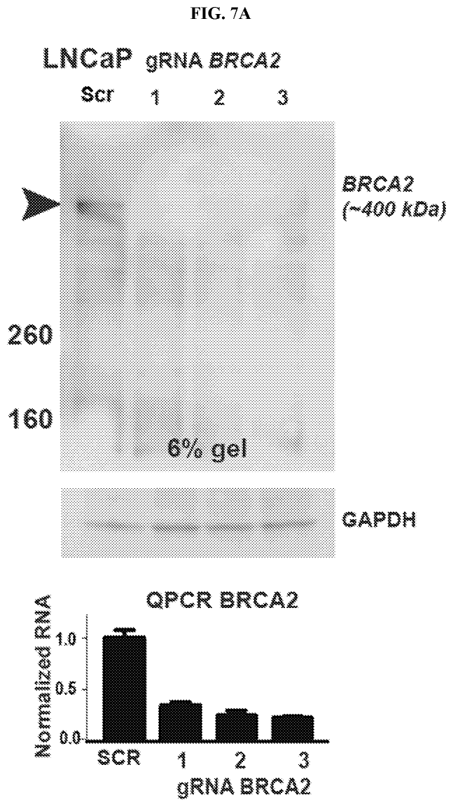

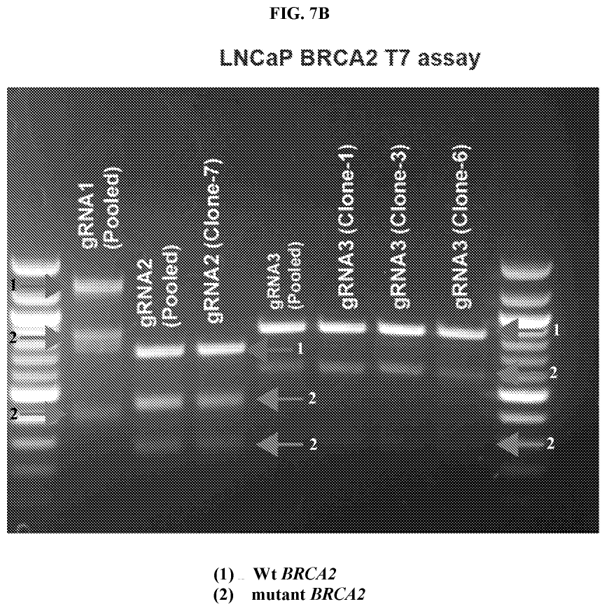

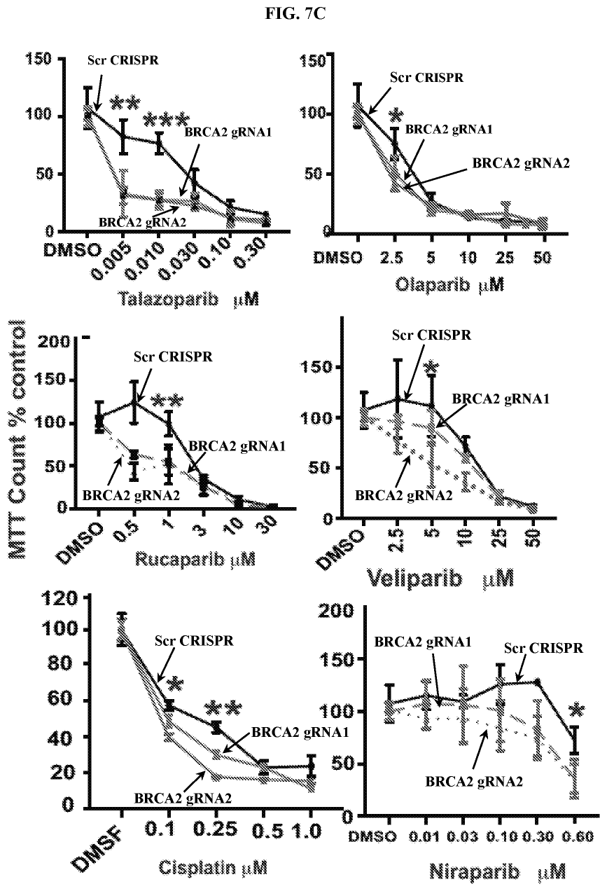

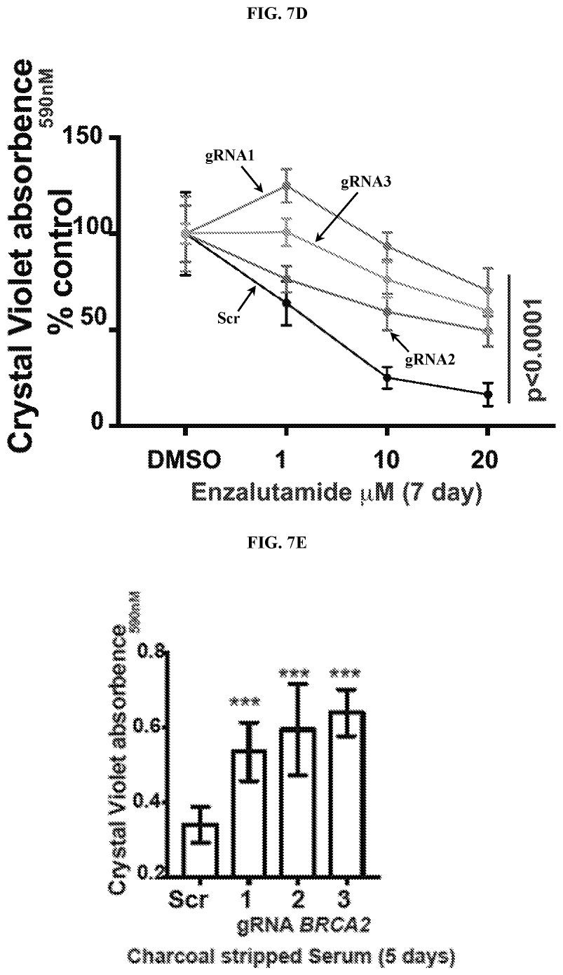

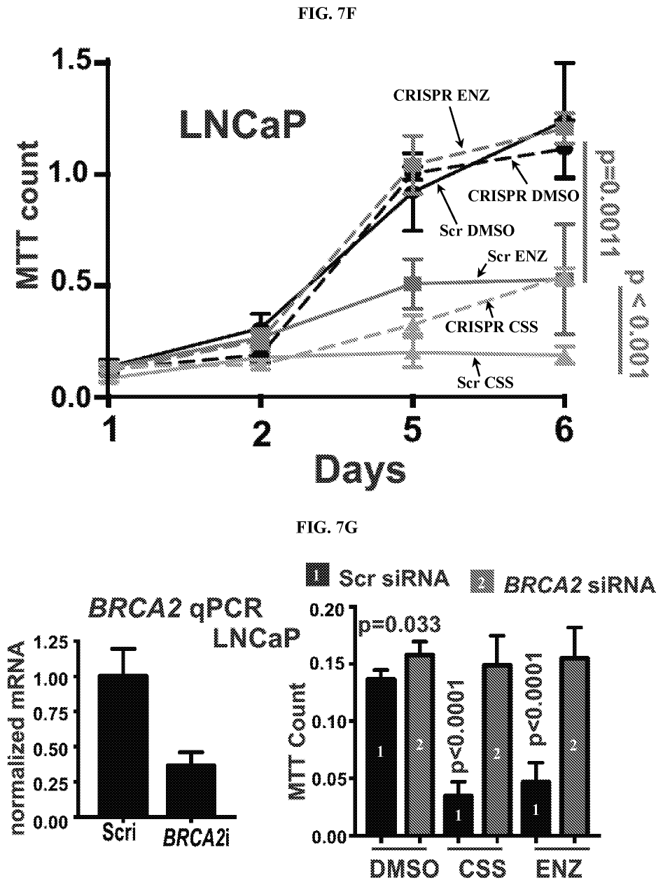

To generate BRCA2 knockout RWPE1 cells, BRCA2 gRNA2 was cloned into LentiCRISPRv2-GFP backbone which constitutively expresses Cas9 and GFP. Lentiviral infected cells were selected by FACS sorting for GFP positive cells (twice) and analyzed by western blot. To generate BRCA2 knockout LNCaP cells by CRISPR/CAS9 methods, LNCaP cells were infected parental with BRCA2 scr gRNA lentivirus, followed by 5 μg/ml puromycin for 5 days. Loss of BRCA2 in the pooled populations of LNCaP cells was analyzed by western blot using BRCA2-specific antibodies and this pooled population of cells was used for the following experiments. For generation of single cell-derived clones, BRCA2 pooled population cells were plated in very low density (500 cells in each 150-mm tissue culture plate in 20 ml of puromycin-supplemented media). After 4 weeks, single cell-derived clones were isolated using PYREX™ cloning cylinders (Fisher Scientific #99-552-21). To determine the genome targeting efficiency of BRCA2 scr gRNA in the pooled population as well as in single cell-derived clones, T7 endonuclease assay was performed using EnGen Mutation detection kit according to manufacturer's protocol (NEB, Ipswich, MA). The primers corresponding to specific gRNA that were used for PCR amplification are listed in FIG. 13. The T7 assay demonstrated a mixed heterozygous population of cells containing wild-type (wt) and mutant BRCA2 DNA (FIG. 7B).

To generate BRCA2-RB1 knockout-knockdown LNCaP cells, parental LNCaP cells were first infected with lentivirus containing BRCA2 gRNA or scr gRNA. Pooled population of the stable cells were established by puromycin selection and analyzed by western blot and qPCR. BRCA2-knockout or scr LNCaP cells were infected with lentivirus containing RB1 shRNA followed by hygromycin selection. BRCA2-knockout or scr (gRNA) LNCaP cells also infected with lentiviral non-targeting shRNA (scr-shRNA) were used as control. Cells within 4-10 passages after stable selection were used for the following experiments.

siRNA or cDNA constructs were transiently transfected in indicated cells using the TransIT-X2 system (Mirus, Madison, WI). A list of CRISPR, cDNA, shRNA, and SMARTpool siRNA constructs is provided in FIG. 13. Efficiency of knockdown and overexpression was verified by qPCR and western blot.

Bioinformatic Analysis of Clinical Cohorts. Bioinformatic analysis of publicly available genomics data from various clinical cohorts was performed using data obtained from cBioPortal (Cerami et al., Cancer Discov 2:401-4 (2012); Gao et al., Sci Signal 6:pl1 (2013)) and Oncomine. Rhodes et al., Neoplasia 6:1-6 (2004). The graphs and Kaplan-Meier survival curves were plotted using GraphPad Prism (version 7, La Jolla, CA). Also used in this study were the cohorts described in the following sources: Armenia et al., Nat Genet 50:645-51 (2018); Baca et al., Cell 153:666-77 (2013); Barbieri et al., Nat Genet 44:685-9 (2012); Beltran et al., Nat Med 22:298-305 (2016); Grasso et al., Nature 487:239-43 (2012); Hieronymus et al., Proc Natl Acad Sci USA 111:11139-44 (2014); Kumar et al., Nat Med 22:369-78 (2016); Robinson et al., Cell 162:454 (2015); Setlur et al., J Natl Cancer Inst 100:815-25 (2008); Taylor et al., Cancer Cell 18:11-22 (2010); Cancer Genome Atlas Research Network, Cell 163:1011-25 (2015); TCGA provisional and pan-cancer prostate, TCGA provisional pan-cancer (unpublished data in cBioPortal); and Zehir et al., Nat Med 23:703-13 (2017).

Western Blot. Cells were washed with HBSS and lysed in radioimmunoprecipitation assay (RIPA) buffer unless otherwise noted (50 mM TRIS-HCl pH 7.4, 150 mM NaCl, 1 mM EDTA, 1% Triton X-100, 1% sodium deoxycholate, and 0.1% SDS) supplemented with protease and phosphatase inhibitors (ThermoFisher Scientific). Protein concentrations were measured using the Bradford protein assay. Western blot was performed using specific antibodies (FIG. 13). For BRCA2 western blot d Novex Tris-Glycine Mini Gels, WedgeWell™ format (6% or 4-20%, ThermoFisher Scientific) were use.

RNA Extraction and qPCR. Total RNA was extracted using the Direct-zol RNA Kit (Zymo Research, Irvine, CA) and reverse transcribed with qScript cDNA SuperMix (Quantabio, Beverly, MA). cDNA corresponding to approximately 10 ng of starting RNA was used for one reaction. qPCR was performed with Taqman Gene Expression Assay (Applied Biosystems, Waltham, MA). All quantifications were normalized to endogenous GAPDH. Probes used for qPCR are listed in FIG. 13.

RNA Sequencing and Pathway Analysis. Total RNA from indicated cells and control LNCaP cells were isolated and analyzed by RNA sequencing by 50 million 2×50 bp reads in the MSK Integrated Genomics Operation Core Facility. RNA sequencing data were analyzed at Partek (St. Louis, MO). Heat maps and volcano plots were developed using Partek manufacturer's instructions. Pathway analysis from RNA sequencing data was performed using gene set enrichment analysis (GSEA) and ToppGene. Chen et al., Nucleic Acids Res 37:W305-11 (2009). The Molecular Signatures Database (MSigDB) is a useful tool to analyze gene set enrichment from the transcriptomic data. Liberzon et al., Bioinformatics 27:1739-40 (2011). Liberzon et al. developed a collection of “hallmarks” gene sets as a part of MSigDB which summarize and represent specific well-defined biological states or processes and display coherent expression. Liberzon et al., Cell Syst 1:417-25 (2015). These “hallmark pathways” summarize information across multiple gene sets and therefore provide more defined biological space for GSEA analysis. Liberzon et al., Cell Syst 1:417-25 (2015). This hallmark signature was used to analyze the RNA sequencing and clinical cohort transcriptome data. Sequencing data are deposited to GEO repository under accession number GSE114155.

For the generation of survival curves using 10-gene (upregulated or downregulated from RNA sequencing) signatures, the Z score for each gene in 10-gene signatures was generated based on the mRNA expression data from the Taylor cohort by using only the subset of primary prostate cancer samples. Taylor et al., Cancer Cell 18:11-22 (2010). mRNA signature score was obtained by summing the Z scores. This generated a unique value for each sample in the cohort; this score was then divided into low and high based on the median. These mRNA scores were then correlated to clinical outcomes in the Taylor cohort. The Kaplan-Meier survival curves were generated and compared using the log-rank test.

3D Matrigel Organoid Assays. 3D organoid assays were performed as previously described. Gao et al., Cell 166:47-62 (2016). Cells were detached using Accutase (Innovative Cell Technologies, San Diego, CA), collected using 70-μm cell strainers, counted (1×10³ cell/well), and re-suspended in serum-free PrEGM BulletKit (Lonza, Morristown, NJ, catalog #CC-3165 & CC-4177) supplemented with 1:50 B-27 supplement (Thermo Fisher Scientific catalog #17504044) and mixed with Matrigel Membrane Matrix (Fisher Scientific CB-40234C) in a 1:1 ratio. The cell and Matrigel mixture were plated on ultra-low attachment plates and allowed to grow for 2 weeks in serum-free PrEGM BulletKit supplemented with 1:50 B-27 medium. Organoids were counted and photographed using GelCount colony counter (Oxford Optronix, Abingdon, England). Organoid diameters more than 100 μm were counted.

Immunofluorescence Study. Cells were plated on cover slips and allowed to grow for 48 hours. Cells were washed with HBSS and fixed in 4% paraformaldehyde for 10 minutes. Cells were permeabilized in 0.2% triton X100 for 20 minutes in room temperature and blocked in blocking solution (2.5% BSA, 2.5% goat and 2.5% donkey serum in HBSS) for 1 hour at room temperature followed by incubation with indicated primary antibody in blocking solution in 4° C. overnight and then secondary antibody for 1 hour at room temperature. For Phalloidin staining, fixed cells were incubated in 1×Alexa Fluor™ 594 Phalloidin (Thermo Fisher Scientific) at 4° C. overnight. Cells were mounted in mounting media containing DAPI and visualized and photographed under a fluorescent microscope.

Cell Proliferation Assay by MTT, BrdU and Crystal Violet. For MTT assay, cells were plated at 2.5×10³ per well in 96-well plates in complete media (10% FBS) or media supplemented with 10% charcoal-stripped serum. Cells were either treated with DMSO or with indicated inhibitors. After indicated times, cells were incubated in 0.5 mg/mL MTT (Invitrogen) for 1 hour at 37° C. MTT crystals were dissolved in isopropanol and absorbance was measured in a BioTek plate reader at 570 nM and represented graphically.

The BrdU assay was performed by BRDU cell proliferation assay kit according to manufacturer's instructions (BrdU cell proliferation assay kit, Cell Signalling #6813). Cells were plated at 2.5×10³ per well in 96-well plates in complete media (10% FBS) or media supplemented with 10% charcoal-stripped serum. Cells were either treated with DMSO or with indicated inhibitors. BrdU incorporation in the proliferating cells was measured in BioTek plate reader at 450 nM and represented graphically. For the Crystal Violet cell proliferation assay, cells (in 96-well plate, treated with indicated drugs or cultured in FBS or CSS supplemented medium) were fixed in chilled 100% methanol for 10 minutes followed by staining with crystal violet (MilliporeSigma) for 2 hours and then washed with water. Crystal violet was dissolved in 1% SDS and absorbance was measured in BioTek plate reader at 595 nM and represented graphically.

Wound Scratch Assay. Control and indicated LNCaP or RWPE1 cells were seeded at a density of 0.5×10⁵ cells per 24-well cell culture plate in complete medium. After 48 hours, a scratch was made with a 10 μL pipette tip in a confluent area of the cell culture dish. Photographs of a selected area of each scratch were taken 48 hours after scratching.

Matrigel Invasion and Boyden Chamber Migration Assay. Matrigel invasion and Boyden chamber migration assays were performed as described earlier. Chakraborty et al., PLoS One 7:e33633 (2012). Briefly, cells in serum-free media (2.5×10³ cells/well for control LNCaP and variants; 1×10³ for PC3M and variants) were added in the top of the Matrigel invasion chamber (Fisher Scientific catalog #08-774-122) or Corning migration chamber (Fisher Scientific catalog #07-200-174). 10% FBS in the lower chamber was used as chemo-attractant. After indicated times, cells in the bottom chamber were fixed in methanol and stained with crystal violet, photographed, and counted under phase-contrast microscopy.

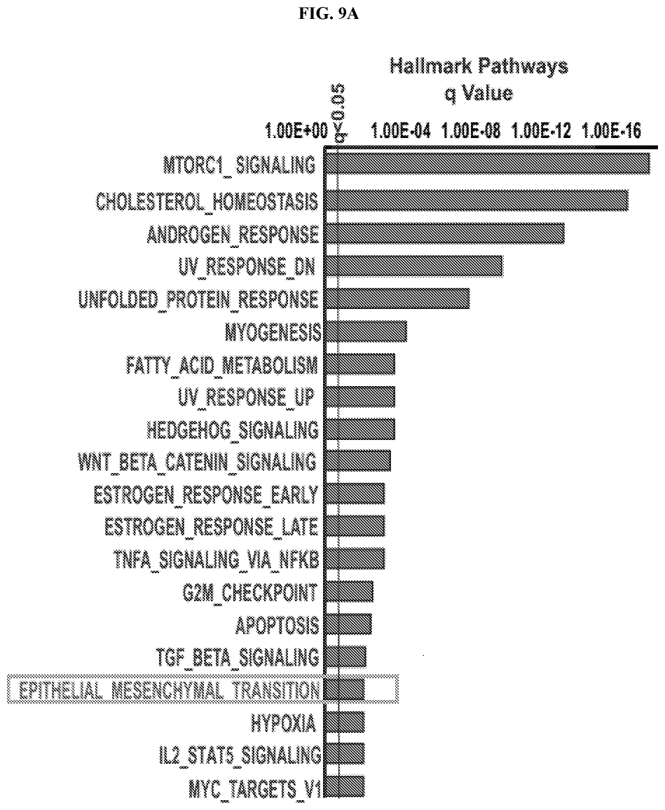

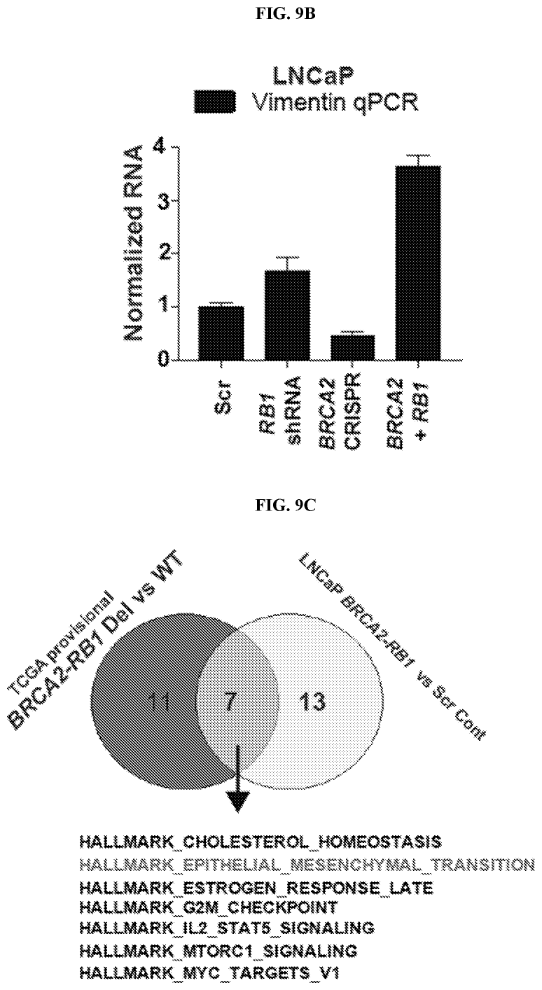

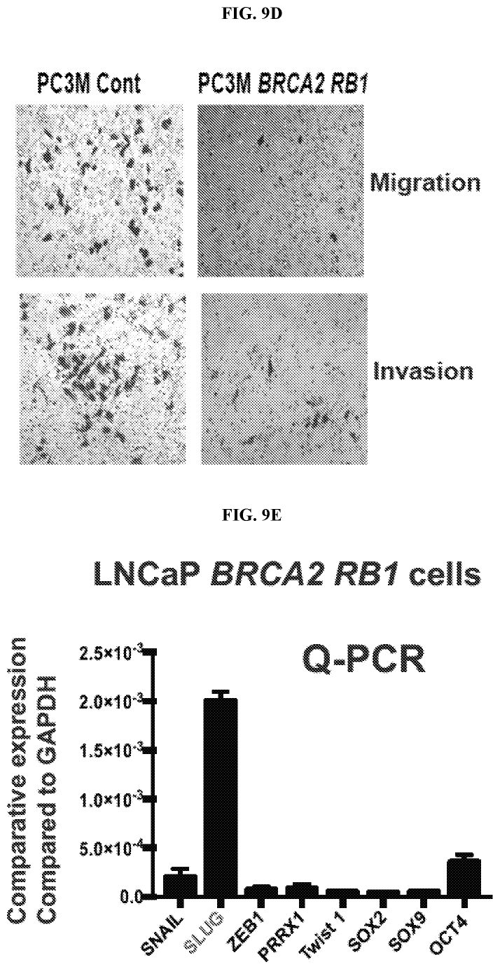

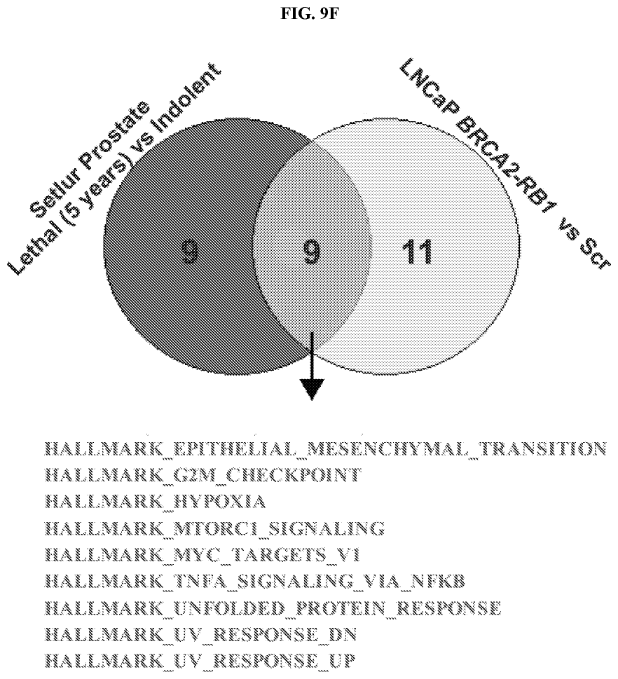

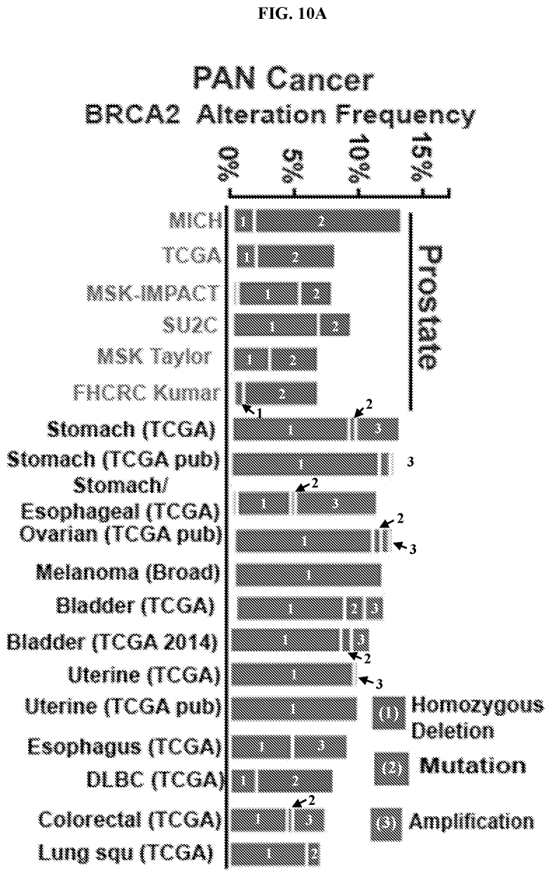

FISH Analysis. All cell lines were harvested and fixed in methanol: acetic acid (3:1). FISH analysis was performed on fixed cells and was based on TCGA data (see, e.g., FIG. 9D). Cancer Genome Atlas Research Network, Cell 163:1011-25 (2015). A 3-color probe was designed to detect loss of BRCA2 (red) and RB1 (orange). Region 13q12 (green) served as the control. The bacterial artificial chromosome (BAC) clones used in the probe-mix were as follows: BRCA2 (RP11-281G19; labelled with red dUTP), RB1 (RP11-305D15; labelled with orange dUTP), and 13q12 (RP11-867N8 and RP11-1031D16; labelled with green dUTP). All RP11 clones were purchased from the Roswell Park Cancer Institute Genomics Shared Resource (Buffalo, NY). Probe labelling, hybridization, post-hybridization washing, and fluorescence detection were performed according to standard laboratory procedures. Prior to hybridization on cell lines, the probe was hybridized on normal peripheral blood (male) and locus specificity was confirmed. Slides were scanned using a Zeiss Axioplan 2i epifluorescence microscope (Carl Zeiss Microscopy, Thornwood, NY) equipped with a 1.4-megapixel CCD camera (CV-M4+CL, JAI, Copenhagen, Denmark) controlled by Isis 5.5.9 imaging software (MetaSystems Group Inc, Waltham, MA).

The entire hybridized area was scanned through a 63× or 100× objective lens to assess quality of hybridization and signal pattern. Following initial scan, for each cell line, a minimum of 100 nuclei were scored and representative cells/regions imaged. A minimum of 25 metaphases were also analyzed and chromosomes counted to infer ploidy. The call for loss was in relation to ploidy; for example, in a near-tetraploid (˜4n) cell line, copy number ≤3 was considered as loss. Three normal lymphoblastoid cell lines (GM06875A, GM07535B, and GM21677), obtained from Corielle Institute (Camden, NJ), were also analyzed and for each cell line, a minimum of 100 nuclei scored to derive the cut-off values (false-positive). The cut-off value for each gene/locus was calculated as the mean of false-positive plus three times the standard deviation and set at 5% for loss (<2 copies) and applicable to diploid cell lines.

Statistical Analysis. Results are reported as mean±SD or ±SEM, unless otherwise noted. Comparisons between groups were performed using an unpaired two-sided Student's t test (P<0.05 was considered significant), unless noted. P-trends were analyzed by one-way ANOVA. Bar graphs were generated using GraphPad Prism software (version 7.0 GraphPad Software, Inc, La Jolla, CA).

### Example 2: Elimination of BRCA2 Leads to Therapy Resistance in Prostate Cancer Cell Lines

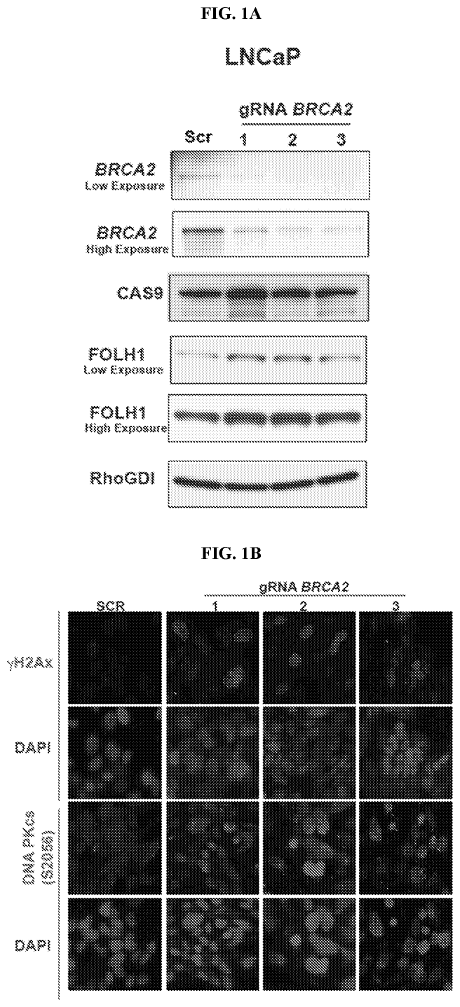

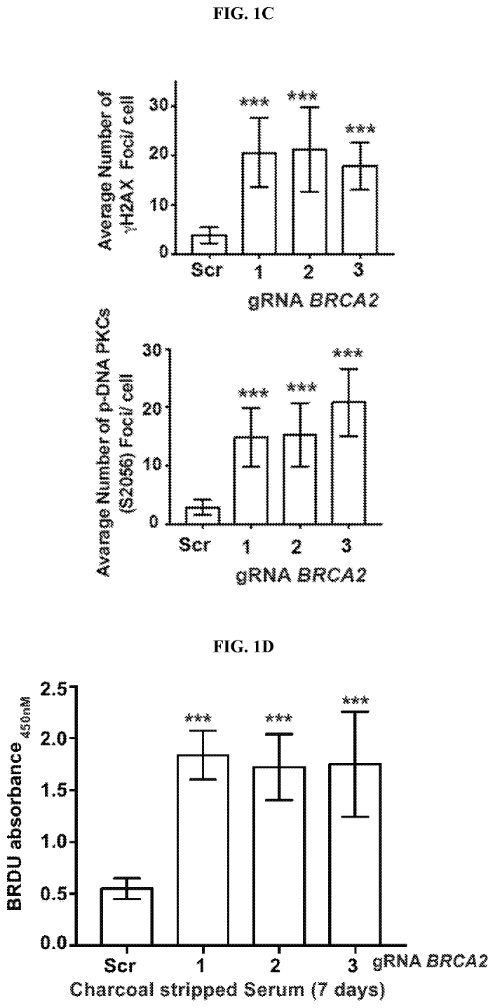

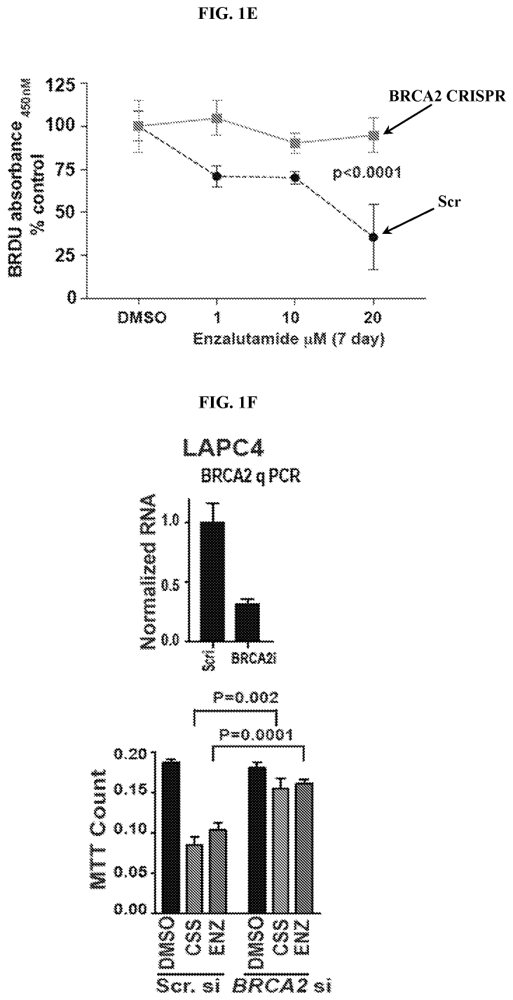

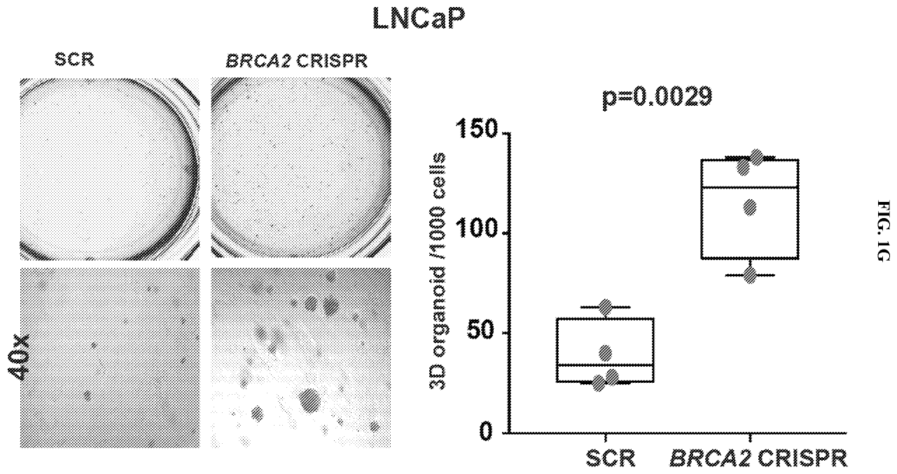

The consequences of BRCA2 deletion were investigated via lentiviral CRISPR/Cas9-mediated stable elimination of BRCA2 in LNCaP cells, a hormone-dependent human prostate cancer cell line. All three gRNAs used herein successfully diminished BRCA2 transcript and protein levels in LNCaP cells (FIGS. 1A, 7A top and bottom panels). Furthermore, the T7 endonuclease assay revealed that all 3 gRNAs induced heterozygous loss of BRCA2 in LNCaP cells (FIG. 7B). As shown in FIG. 7C, BRCA2-null LNCaP cells exhibited enhanced sensitivity to various PARPi and cisplatin. However, the data also showed that BRCA2 knockout LNCaP cells exhibited more sensitivity towards talazoparib (BMN 673) and rucaparib compared to control gRNA (scr) infected cells (FIG. 7C). Higher expression of FOLH1 was detected in BRCA2 knockout LNCaP cells compared to control cells (FIG. 1A). It was observed that elimination of BRCA2 increased phosphorylation of γH2AX in LNCaP cells (FIGS. 1B top panel, 1C top panel), a biomarker for defective repair of double-strand breaks, indicating that CRISPR-mediated elimination of BRCA2 may induce a defect in homologous recombination repair in LNCaP cells. An increase in S2056 autophosphorylation of DNA-PKcs was also observed in BRCA2 knockout LNCaP cells, indicating hyperactivation of DNA-PKcs (FIGS. 1B bottom panel, 1C bottom panel). Furthermore, as shown in FIGS. 1D, 7E and 7F, BRCA2-null LNCaP cells exhibited androgen-independent growth, as evidenced by enhanced 2D growth in androgen-deprived charcoal-stripped medium compared to control LNCaP. Also, the BRCA2-null LNCaP cells exhibited relative resistance to enzalutamide (FIGS. 1E, 7D and 7F), indicating that these cells became castration-resistant. Similarly, RNAi-mediated transient silencing of BRCA2 in LNCaP and LAPC4 (another androgen-dependent human prostate cancer cell line) cells also exhibited resistance to androgen depletion, as evidenced by growth in charcoal-stripped medium or complete media supplemented with enzalutamide (FIGS. 7G and 1F). As shown in FIG. 1G, BRCA2-null LNCaP cells also exhibited enhanced prostatosphere formation in 3D Matrigel cultures (organoids) in the androgen-independent condition, indicating that BRCA2-null LNCaP cells are more tumorigenic compared to control LNCaP cells.

These results demonstrate that BRCA2-mutant prostate cancer cells show defective double-strand break repair, castration-resistance, and an invasive phenotype.

### Example 3: Concomitant Elimination of BRCA2 and RB1 Induces an Invasive Phenotype in Human Prostate Cancer Cells

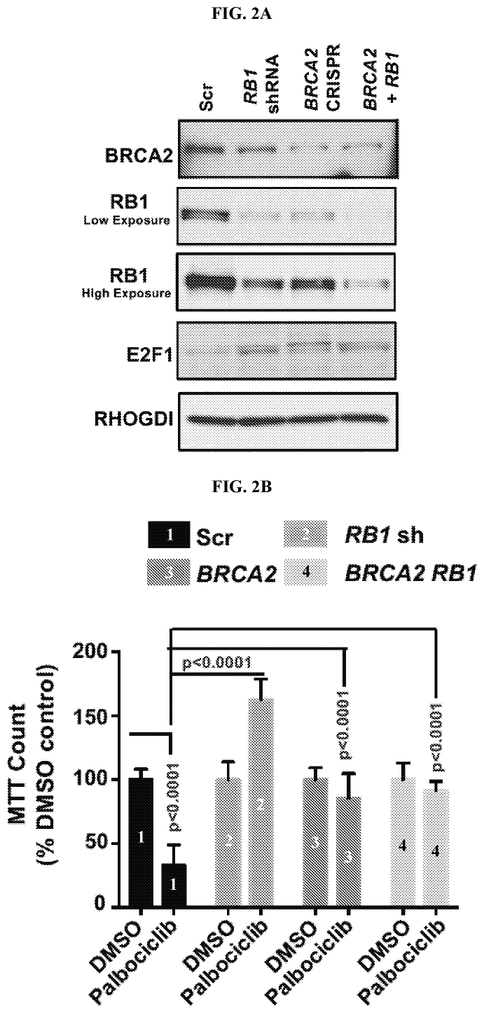

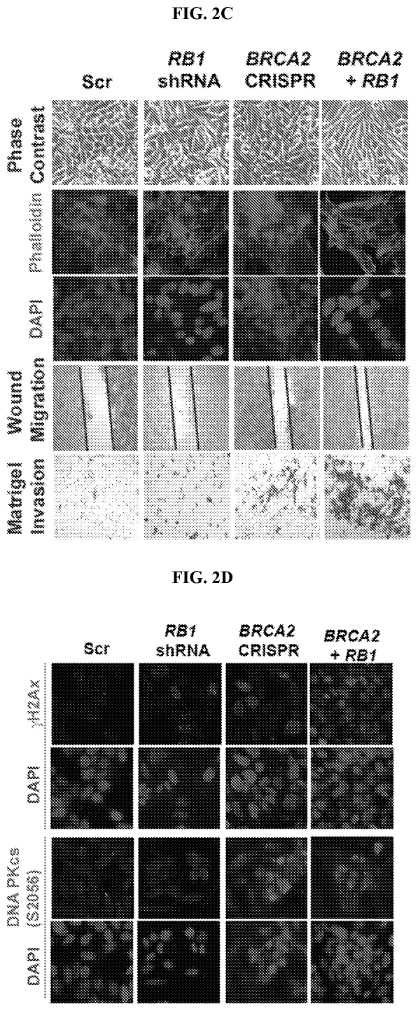

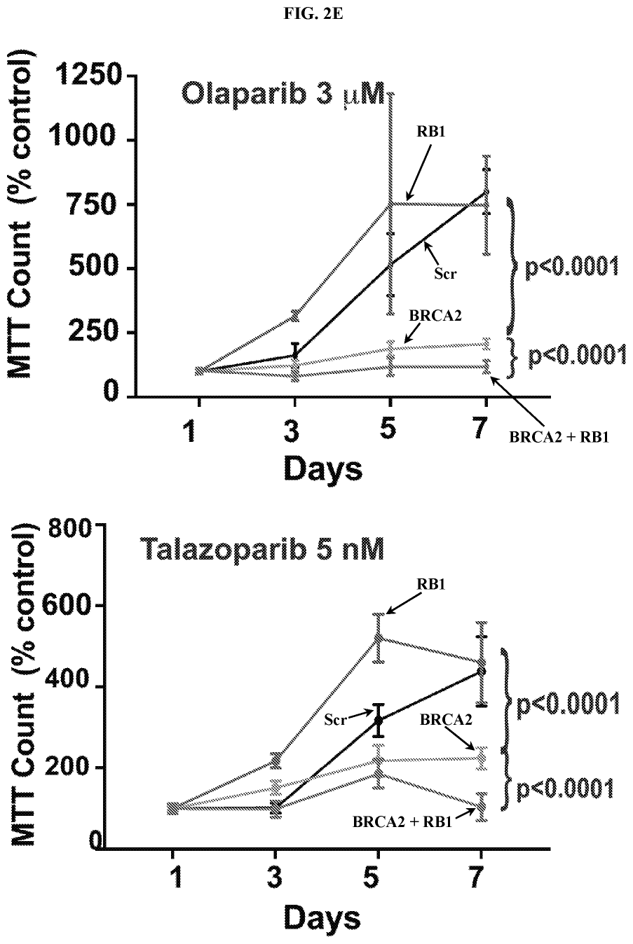

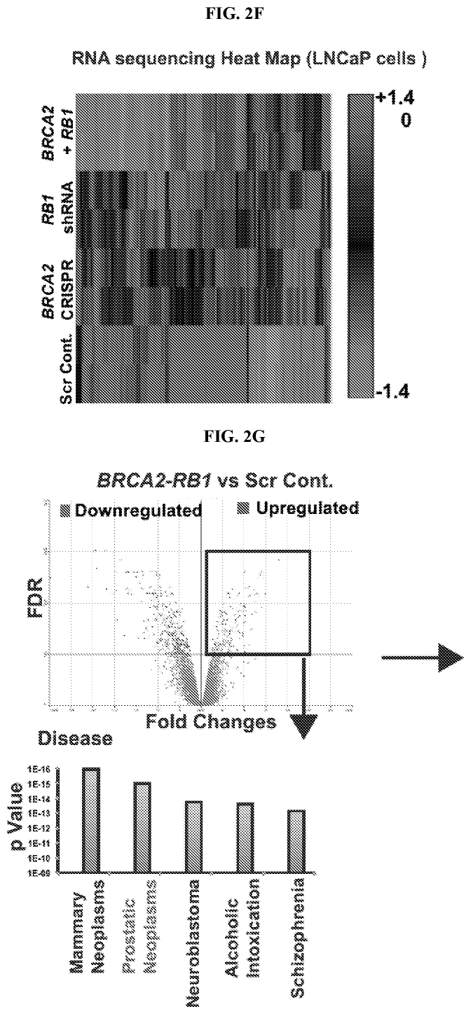

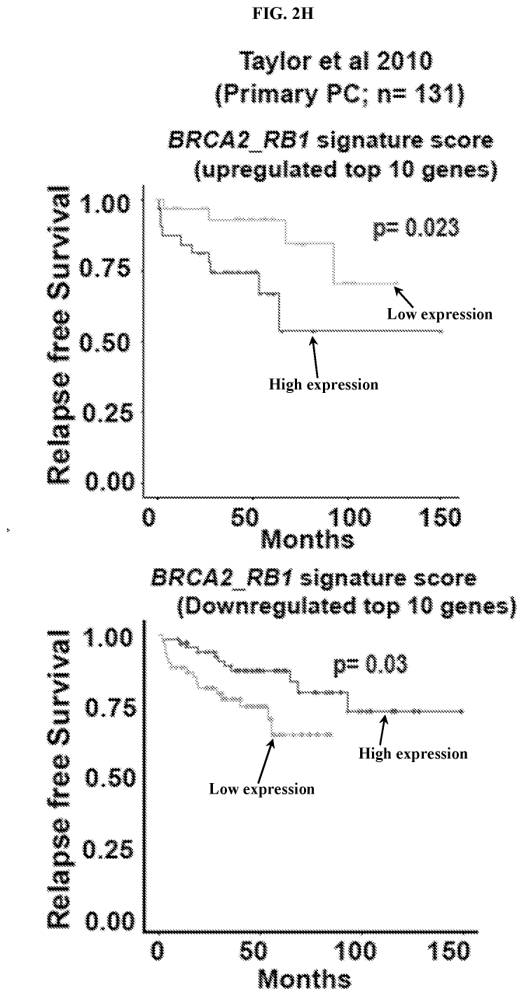

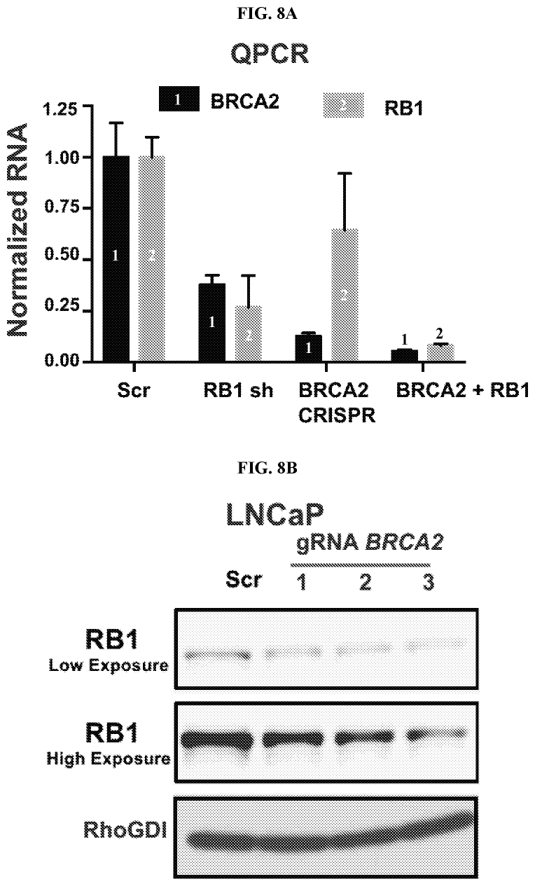

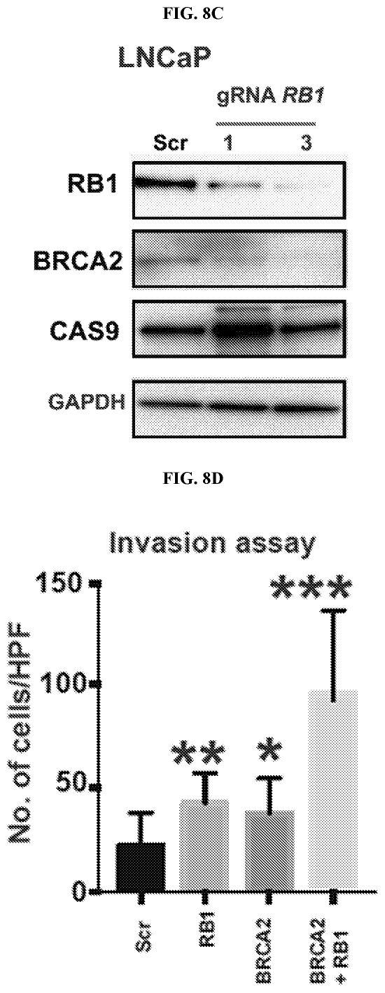

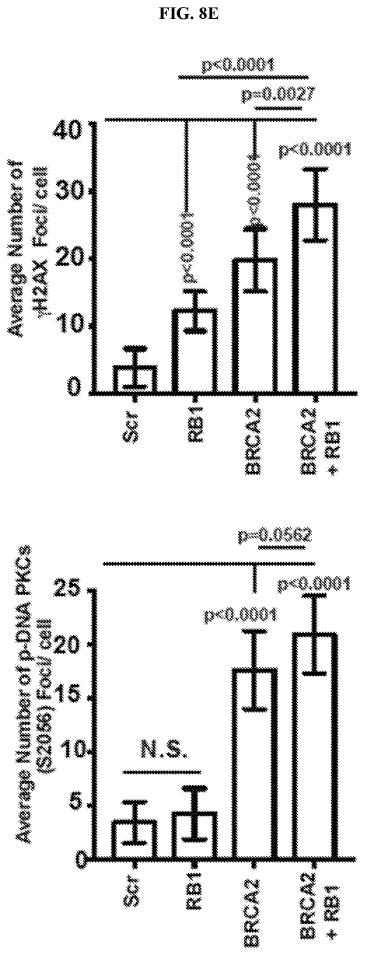

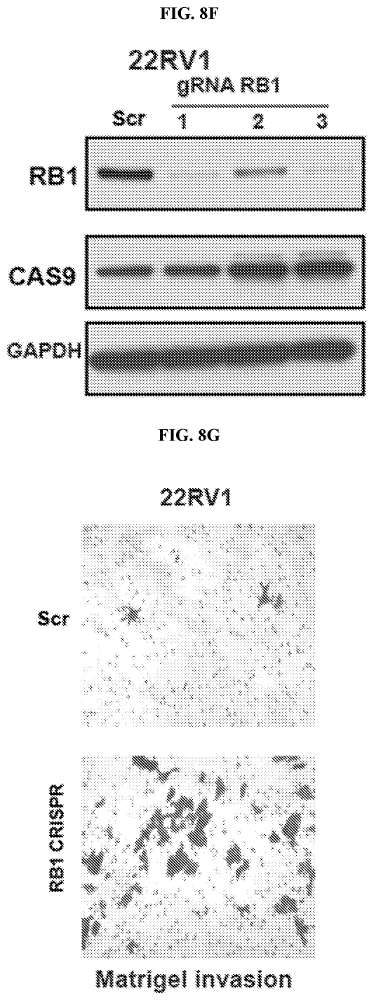

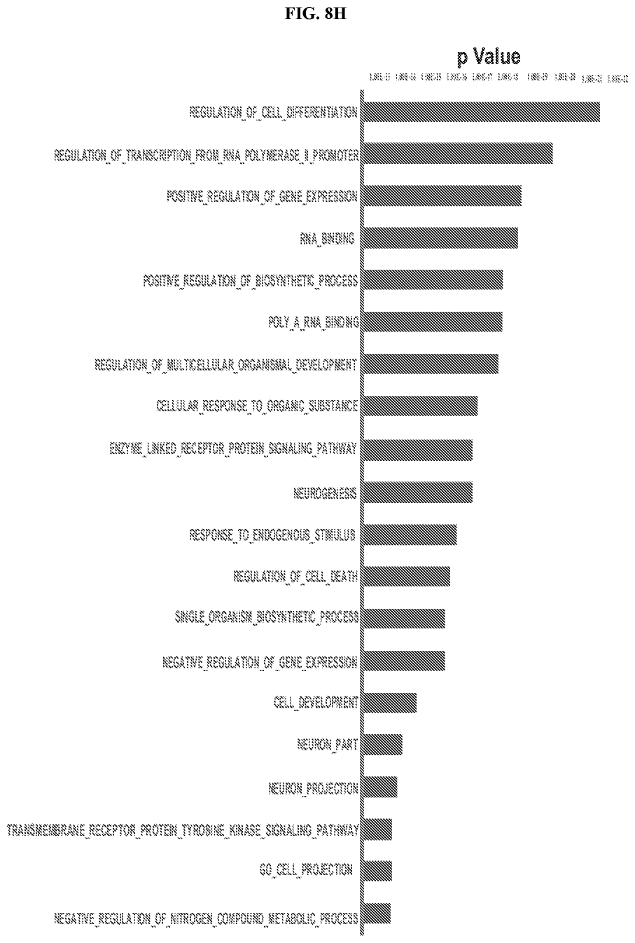

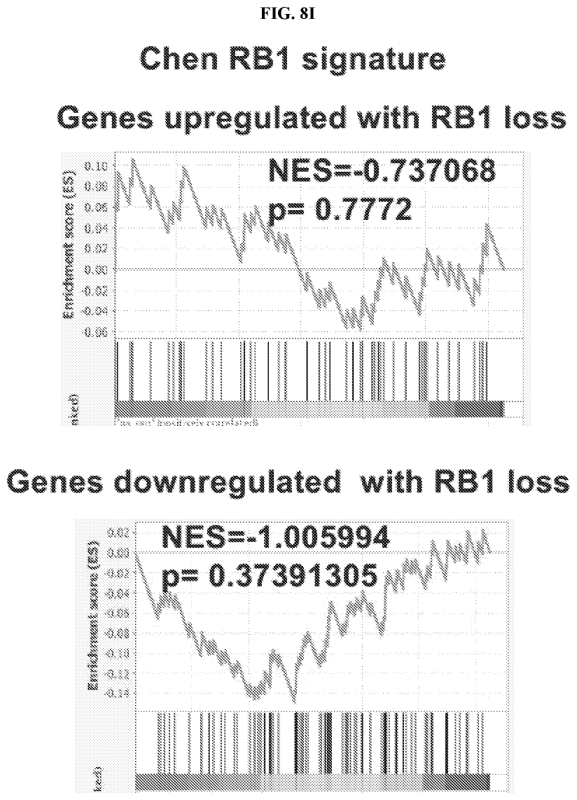

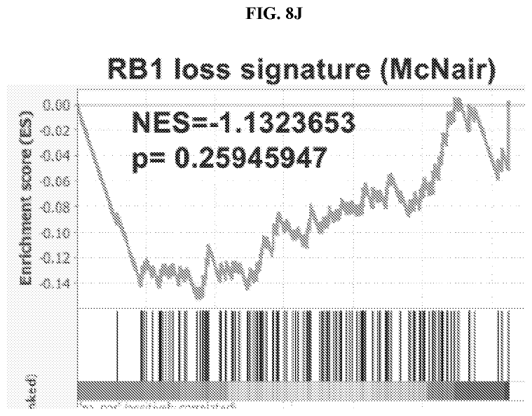

To investigate the direct effect of the BRCA2-RB1 co-deletion on human prostate cancer cells, a shRNA against RB1 (shRB1; in a lentiviral stable expression vector) was introduced into BRCA2-null LNCaP cells, generating BRCA2-RB1 knockdown LNCaP cells (hereafter “LNCaP-BRCA2-RB1”). As shown in FIGS. 2A and 8A, downregulation of BRCA2 protein and mRNA was observed in RB1 knockdown LNCaP cells. Interestingly, it was also observed that loss of BRCA2 attenuated RB1 protein expression in all BRCA2 knockout LNCaP cells (FIGS. 2A and 8B). Similarly, as shown in FIG. 8C, CRISPR-mediated knockout of RB1 also inhibited BRCA2 expression in LNCaP cells, indicating a possible feed-forward loop between BRCA2 and RB1 expression in prostate cancer cells. Induction of E2F-1 was observed in RB1 and/or BRCA2 knockdown/knockout cells (FIG. 2A). Furthermore, BRCA2-RB1 knockout/knockdown LNCaP cells exhibited relative resistance to the CDK4/6 inhibitor palbociclib as determined by MTT assay, as shown in FIG. 2B. These data suggested that depletion of RB1 and/or BRCA2 in LNCaP cells is sufficient to induce canonical downstream pathway suppression by RB1.

As shown in FIG. 2C, LNCaP-BRCA2-RB1 cells exhibited elongated morphology. Immunofluorescence staining using phalloidin showed the remodeling of actin filaments in LNCaP-BRCA2-RB1 cells, further supporting the changes of cellular morphology upon co-loss of BRCA2 and RB1 (FIG. 2C). LNCaP-BRCA2-RB1 cells also exhibited enhanced wound migration and invasion through Matrigel, as shown in FIGS. 2C and 8D. Knockdown/knockout of either RB1 or BRCA2 alone induced an intermediate invasive phenotype (FIGS. 2C and 8D).

As shown in FIGS. 2D and 8E, increased phosphorylation of γH2AX was observed in LNCaP-BRCA2-RB1 cells compared to BRCA2 or RB1 knockout/knockdown LNCaP cells. Furthermore, a very modest increase of S2056 autophosphorylation of DNA-PKcs was observed in LNCaP-BRCA2-RB1 cells compared to BRCA2 knockout LNCaP cells (FIGS. 2D and 8F). RB1 loss alone only caused a modest increase of phosphorylation of γH2AX but not S2056 autophosphorylation of DNA-PKcs compared to control LNCaP cells, as shown in FIGS. 2D and 8F. As shown in FIG. 2E, treatment with the PARPi olaparib and talazoparib caused more cell growth inhibition in LNCaP-BRCA2-RB1 cells than on BRCA2-null LNCaP cells. Any inhibitory effect of olaparib or talazoparib on RB1 knockdown cells could not be detected compared to control LNCaP cells. These data suggested that co-loss of BRCA2 and RB1 increases sensitivity to PARPi in prostate cancer cells compared to BRCA2 loss alone. In contrast, RB1 loss alone was not associated with sensitivity of prostate cancer cells to PARPi (FIG. 2E).

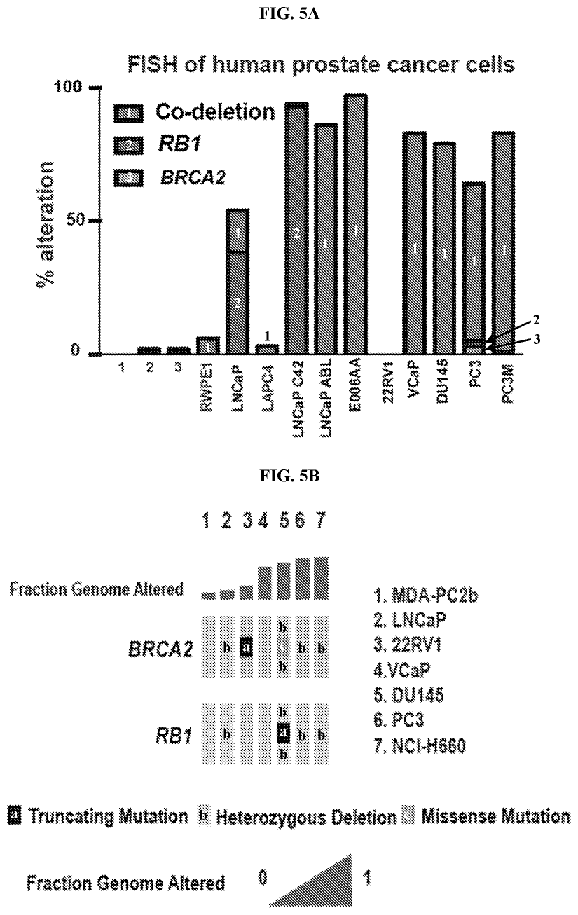

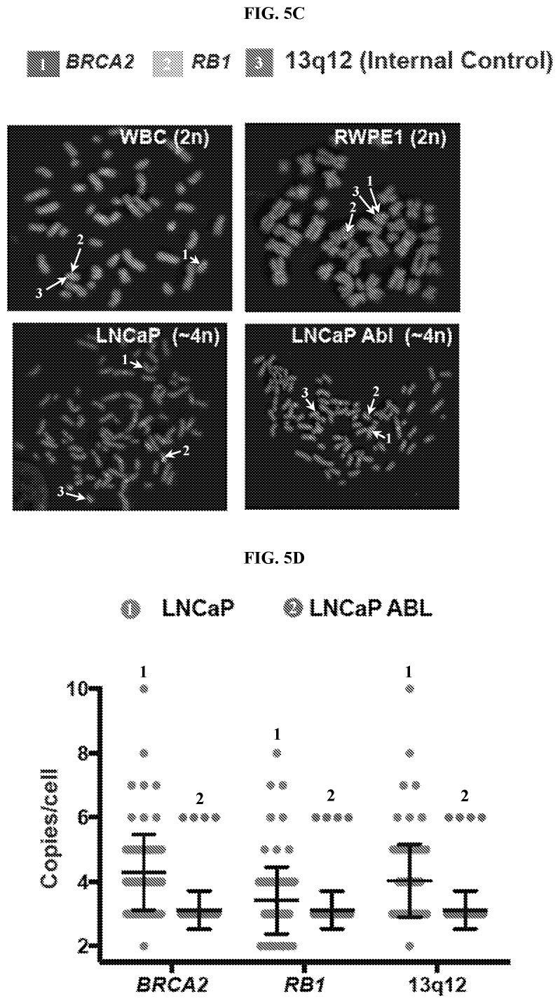

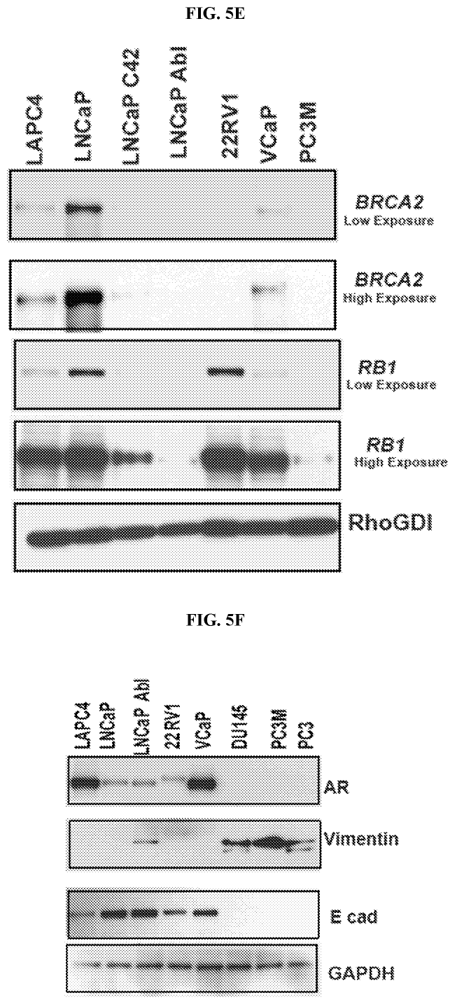

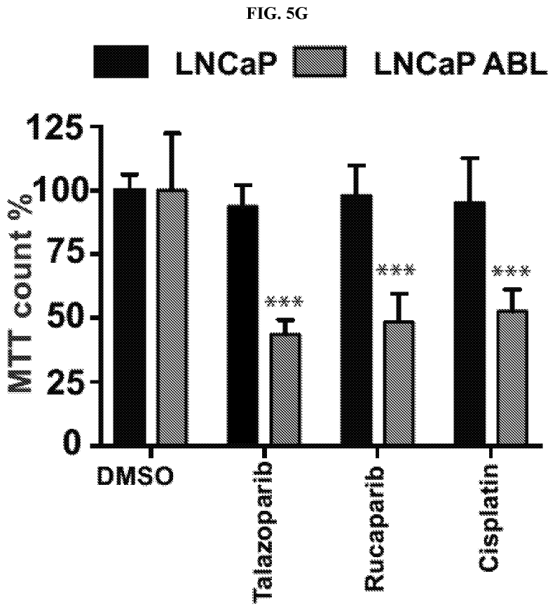

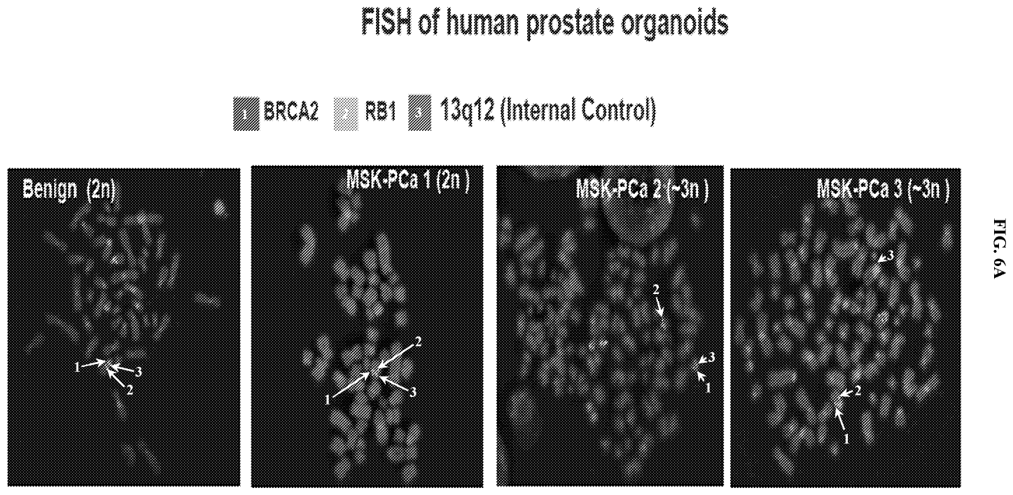

To further confirm the effect of co-loss of BRCA2 and RB1 on the invasive phenotype of prostate cancer cells, RB1 was knocked out in 22RV1 cells which harbor oncogenic mutation of BRCA2 (T3033Nfs*11; FIG. 5B). As shown in FIG. 8G, RB1 knockout 22RV1 cells exhibited higher Matrigel invasion compared to control 22RV1 cells.

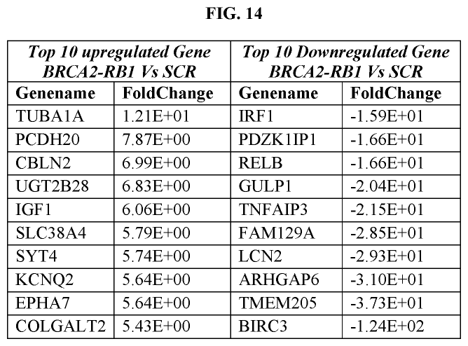

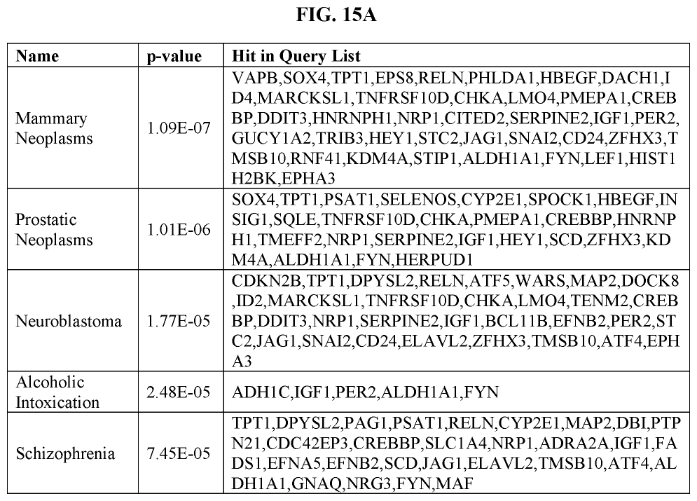

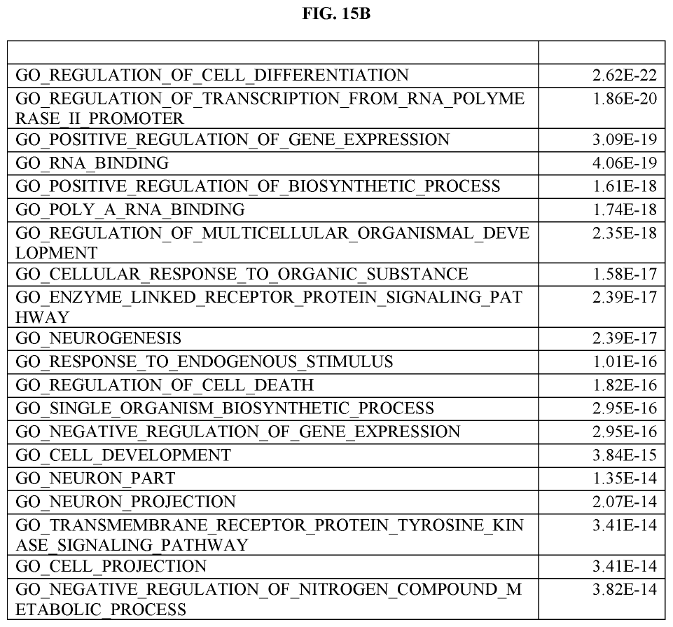

To understand the molecular consequence of BRCA2-RB1 loss, RNA sequencing from the LNCaP-BRCA2-RB1 cells was performed. Interestingly, a gradation of changes in gene expression was observed in these cells compared to knockdown of either BRCA2 or RB1 alone, which provided further evidence of an additive effect of BRCA2-RB1 co-loss in LNCaP cells (FIG. 2F). As shown in FIG. 2G (top and bottom panels), pathway analysis of upregulated genes in LNCaP-BRCA2-RB1 cells showed that the gene signature was prostate cancer-specific (FIGS. 15A-15B). Using single-sample GSEA (ssGSEA), a 10-gene signature was developed from the 10 mRNAs most upregulated and most downregulated by co-loss of BRCA2 and RB1 in LNCaP cells, as shown in FIG. 14. Both 10-gene signatures strongly predicted early relapse in localized prostate cancer in the Taylor cohort (FIG. 2H). In addition, GSEA was performed on the upregulated transcriptome of LNCaP-BRCA2-RB1 cells (FIGS. 8H and 15A-15B) and induction of several essential molecular pathways, including regulation of cell differentiation and transcription, were observed to be enriched upon co-loss of BRCA2 and RB1 in LNCaP cells. However, any correlation between previously published RB1 signatures (McNair et al., J Clin Invest. 128(1):341-58 (2018); Chen et al., Cancer Research 25(14):4290-9 (2019)) and the LNCaP cell-derived BRCA2-RB1 signature could not be to detected (FIGS. 8I and 8J)

These results demonstrate that BRCA2-RB double mutant prostate cancer cells show an invasive phenotype. These results also show that BRCA2-RB double mutant cancer cells are more sensitive to the PARP inhibitors of the present technology than BRCA2 or RB single mutant. Accordingly, the methods disclosed herein are useful for selecting a prostate cancer patient for treatment with a PARP inhibitor, and/or treating or preventing metastatic castration-resistant prostate cancer in a patient in need thereof.

### Example 4: Co-Elimination of BRCA2 and RB1 Leads to EMT

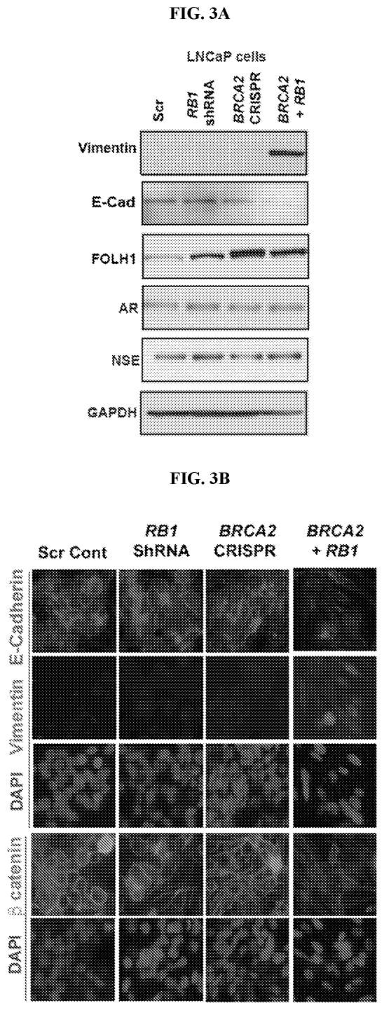

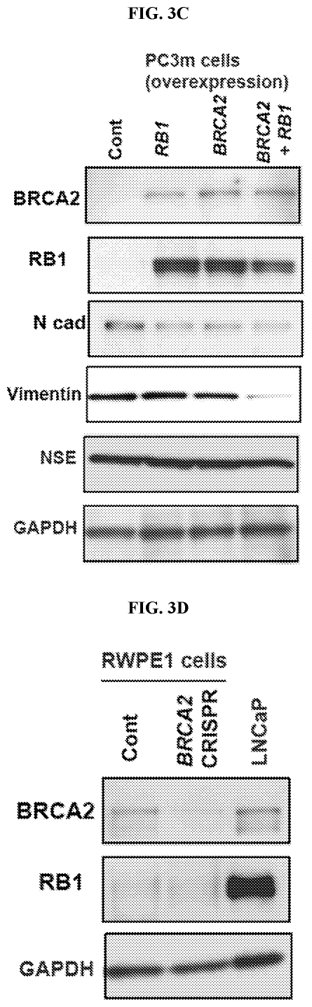

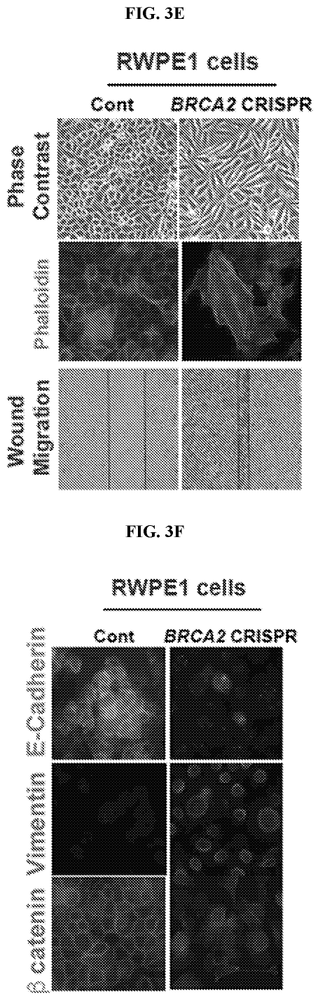

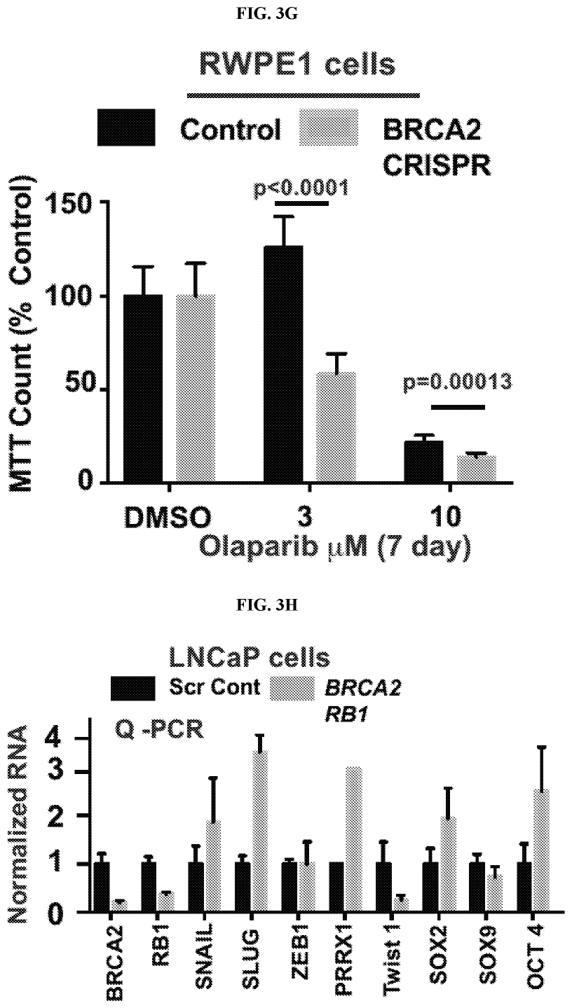

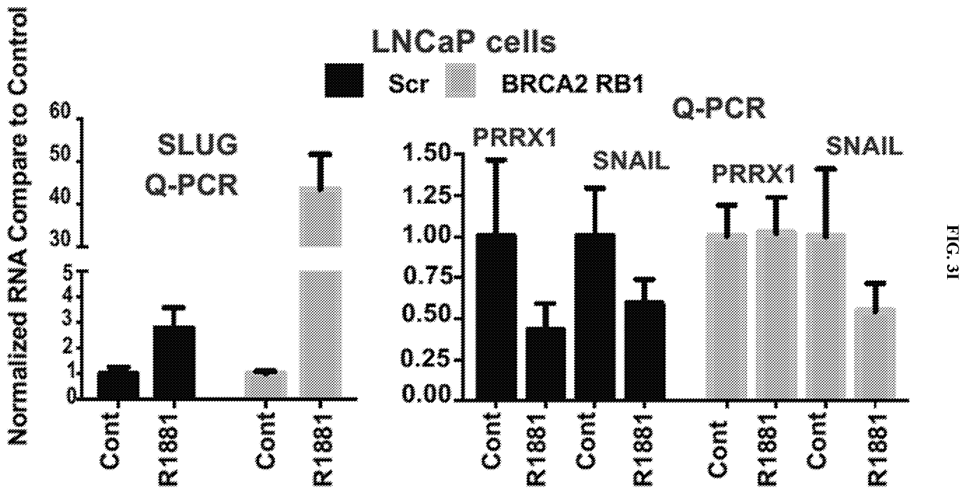

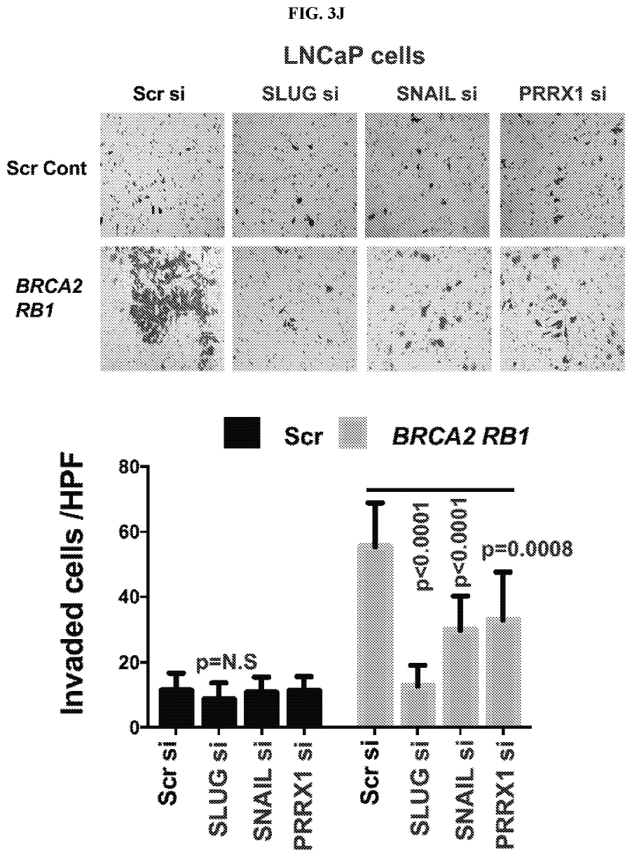

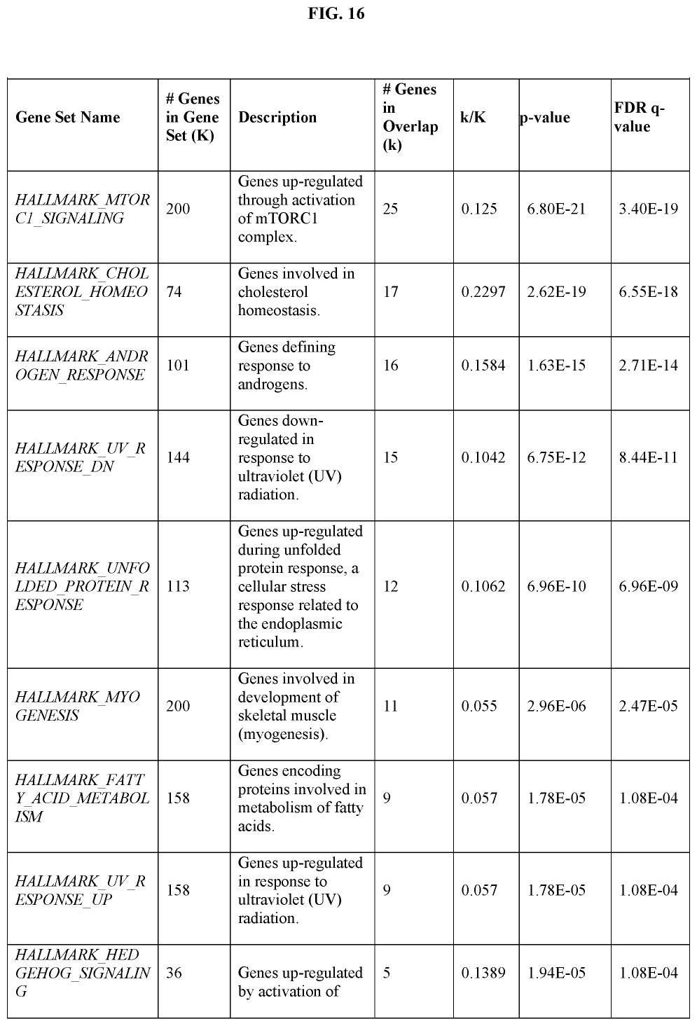

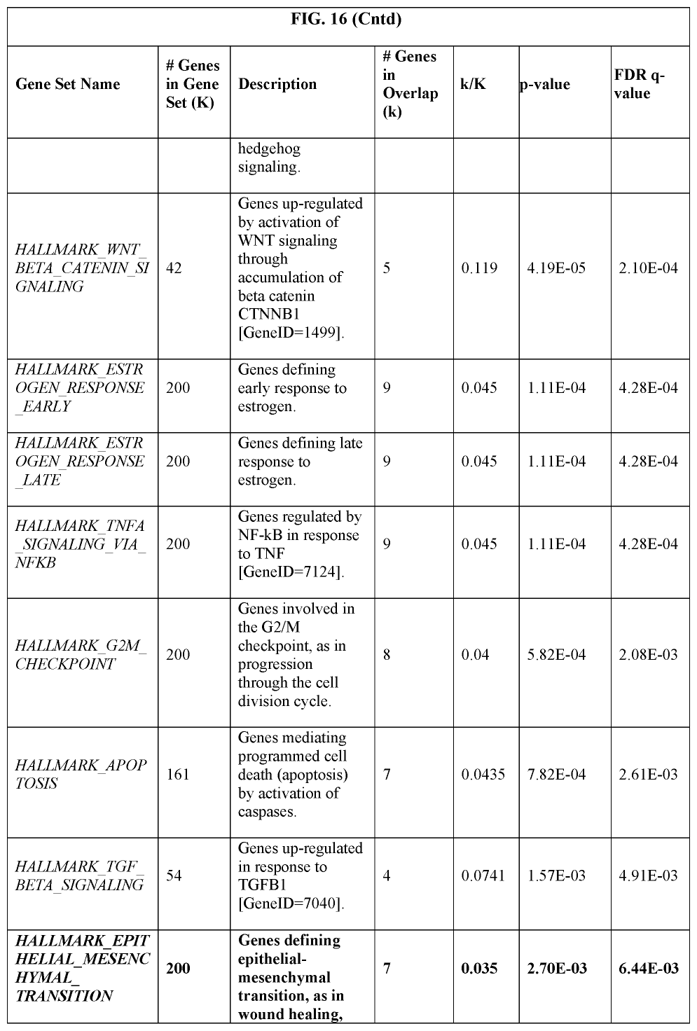

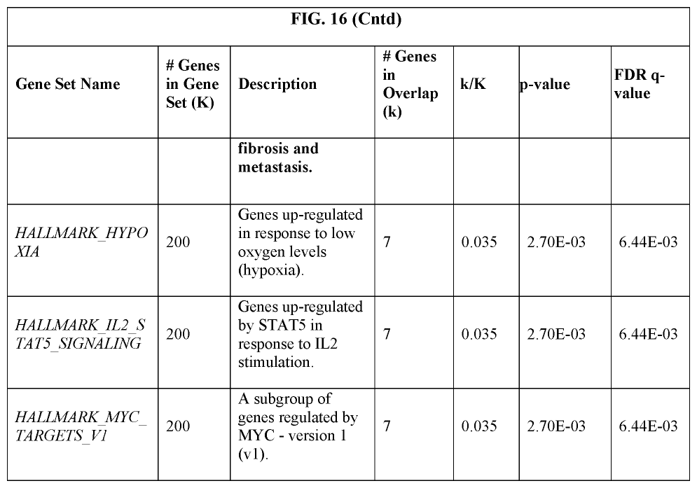

These observations prompted investigation of the molecular mechanism by which the invasive phenotype resulting from co-loss of BRCA2 and RB1 in LNCaP cells occurs. The “hallmark pathways” analysis was performed using GSEA in the upregulated transcriptome of LNCaP-BRCA2-RB1 cells (FIG. 2G top panel). As shown in FIGS. 9A and 16, an increased expression of several EMT and de-differentiation-related signaling pathways (mTORC1, Hedgehog, TNFα-NFKB, TFGβ) was observed, including enrichment of the hallmark EMT signaling pathway. Decreased expression of E-cadherin and increased expression of the mesenchymal marker vimentin (both translational and transcriptional) was observed in the double knocked down cells compared to control LNCaP cells (FIGS. 3A and 9B). As shown in FIG. 3B, immunofluorescence staining also showed loss of cell membrane E-cadherin and β-catenin and gain of vimentin in the LNCaP-BRCA2-RB1 cells. These observations are consistent with the elongated morphology and actin cytoskeleton remodeling of LNCaP-BRCA2-RB1 cells shown in FIG. 2C. Moreover, these findings further supported the observation that LNCaP-BRCA2-RB1 cells undergo an EMT-like transformation, while knockdown of BRCA2 or RB1 alone induce a partial EMT-like phenotype (FIGS. 3A-3B). However, any changes in expression of AR or the neuroendocrine marker NSE were not detected in double knockout/knockdown LNCaP cells compared to control cells (FIG. 3A).

BRCA2 and RB1 was overexpressed in highly aggressive mesenchymal-like PC3M cells which exhibit low endogenous BRCA2 and RB1. As shown in FIG. 3C, overexpression of BRCA2 and RB1 inhibited vimentin and N-cadherin expression in PC3M cells; however, NSE expression remains unchanged. Interestingly, it was also observed that overexpression of either of the genes (BRCA2 or RB1) auto-regulated the expression of the other in PC3M cells (FIG. 3C), further indicating the feed-forward loop between BRCA2 and RB1 in prostate cancer. BRCA2 and RB1 also exhibited diminished Boyden chamber migration and Matrigel invasion in overexpressed PC3M cells compared to control cells, as shown in FIG. 9D.

To further validate whether loss of BRCA2 and RB1 is sufficient to induce EMT in prostate cancer cells, the immortalized benign human prostate cells RWPE1 were used. RWPE1 cells express significantly lower RB1 protein compared to parental LNCaP cells due to their expression of a single copy of human papilloma virus 18 (HPV 18) (FIG. 3D). CRISPR was used to knockout BRCA2 from RWPE1 cells (FIG. 3D) and BRCA2 knockout RWPE1 cells exhibited elongated morphology and remodeling of actin filament (FIG. 3E). As shown in FIG. 3E, enhanced wound migration was also observed in BRCA2 knockout RWPE1 cells. Immunofluorescence staining also showed loss of cell membrane E-cadherin and β-catenin and gain of vimentin in the BRCA2-knockout RWPE1 cells as shown in FIG. 3F. As shown in FIG. 3G, BRCA2-null RWPE1 cells also exhibited enhanced sensitivity to PARPi olaparib.

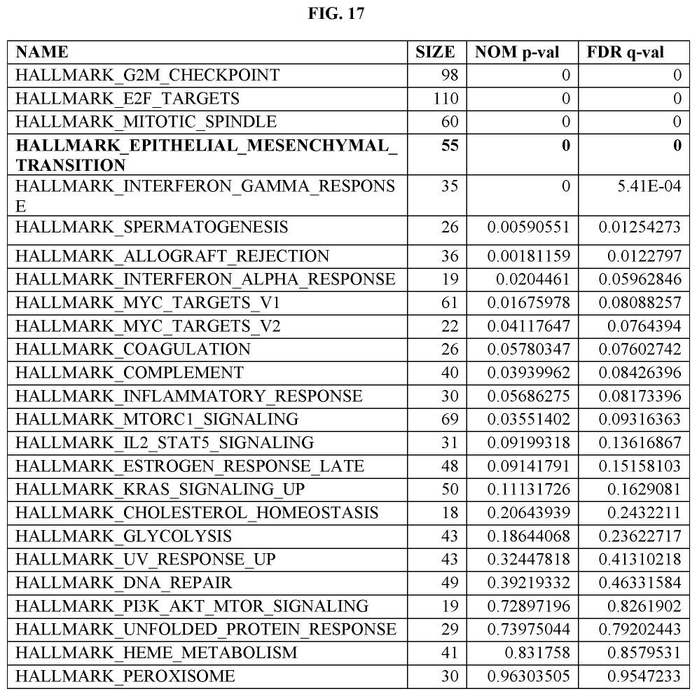

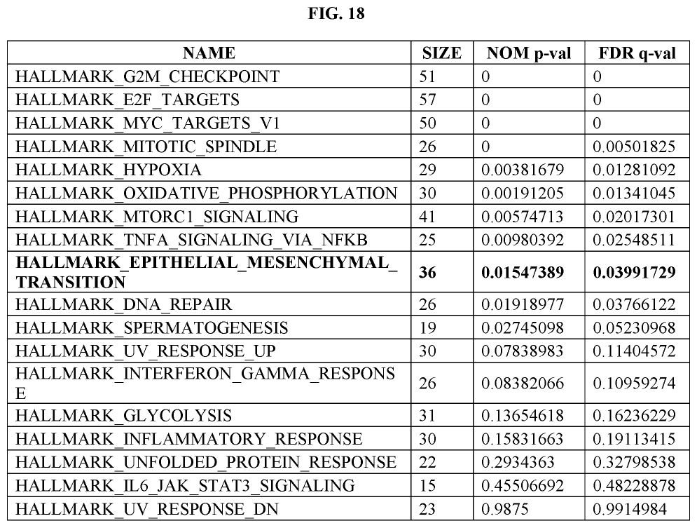

The transcriptome that is enriched in the BRCA2-RB1 co-deleted TCGA provisional prostate cancer cohort was analyzed and GSEA hallmark pathway analyses were performed (FIG. 17 and data not shown). As shown in FIGS. 9C and 17, EMT was observed to be one of the common pathways enriched in the BRCA2-RB1-null cell line and TCGA cohort. More importantly, the analysis of the Setlur prostate cancer cohort (lethal vs indolent) using Oncomine suite and GSEA also demonstrated enrichment (P=0.015, q [adjusted P-value based on false discovery rate (FDR)]=0.039, normalized enrichment score [NES]=1.764) of the EMT pathway (FIGS. 9F and 18), indicating the clinical significance of EMT in lethal prostate cancer.

To determine which transcriptional factors were involved in EMT transformation, the expression of previously demonstrated EMT-related transcription factors was analyzed by qPCR. As shown in FIG. 3H, upregulation of EMT transcription factors SLUG (SNAI2) and SNAIL (SNAI1) and transcriptional co activator PRRX1 was observed in LNCaP-BRCA2-RB1 compared to LNCaP cells. Relative SLUG expression was significantly (>100-fold) higher compared to other EMT transcription factors in LNCaP-BRCA2-RB1 cells (FIG. 3I). Previously SLUG had been demonstrated as an androgen-regulated transcription factor which facilitates castration resistance in prostate cancer. Wu et al., Mol Endocrinol. 26(9):1496-507 (2012). Accordingly, it observed that treatment with androgen (R1881) significantly increased SLUG, but not SNAIL or PRRX1 mRNA in LNCaP-BRCA2-RB1 cells as shown in FIG. 3I. As shown in FIG. 3J, siRNA-mediated knockdown of SLUG, SNAIL or PRRX1 showed inhibition of invasiveness compared to control siRNA-transfected LNCaP-BRCA2-RB1 cells or control (scr) LNCaP cells.

These results demonstrate that BRCA2-RB mutant prostate cancer cells show an upregulation of EMT transcription factors.

### Example 5: Frequent Deletion of BRCA2 in Prostate Cancer

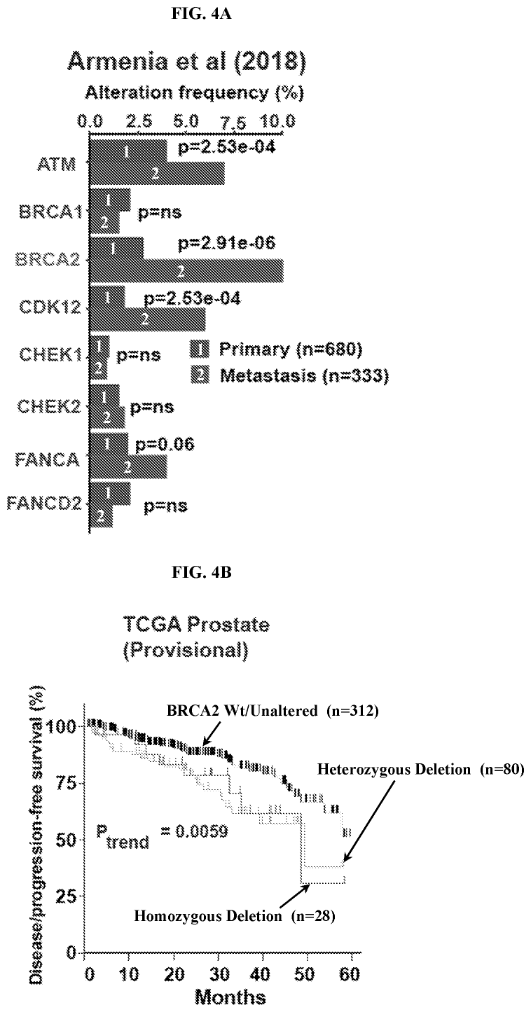

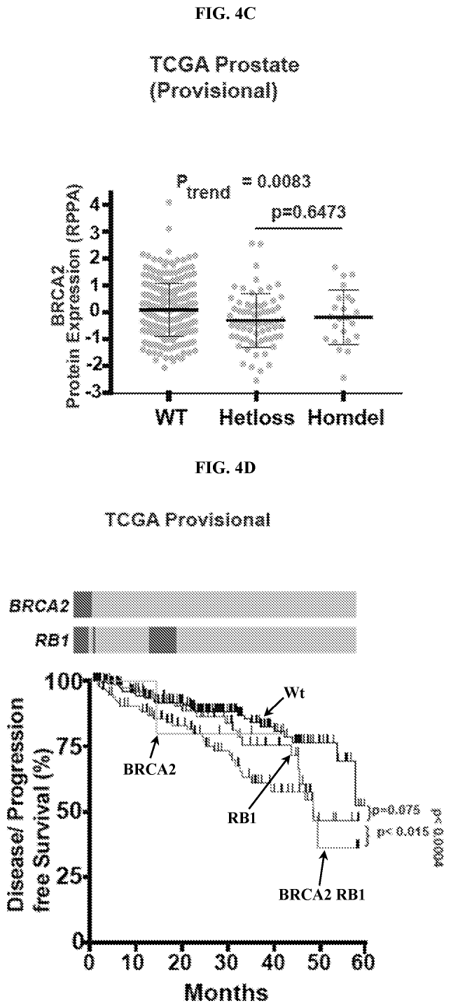

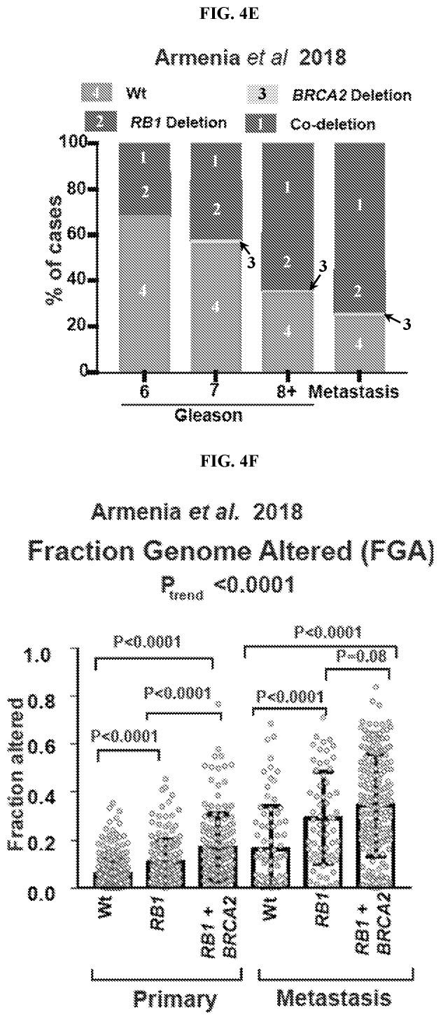

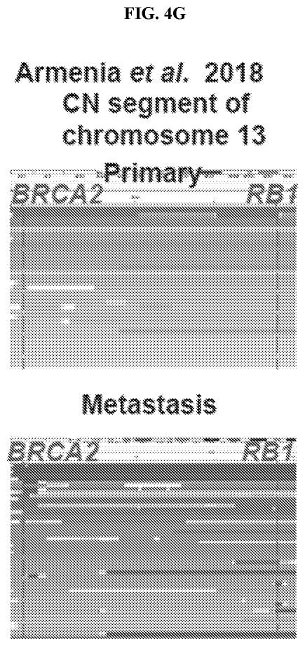

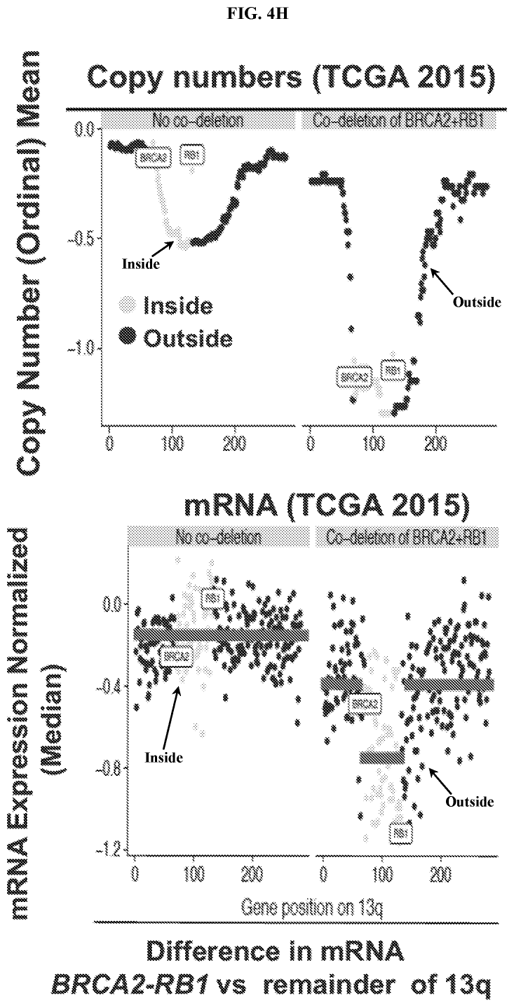

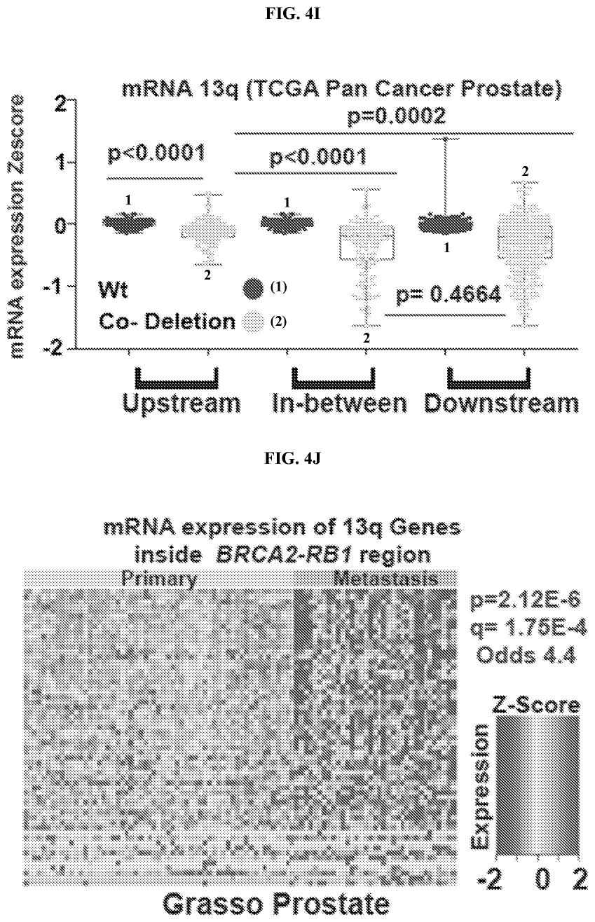

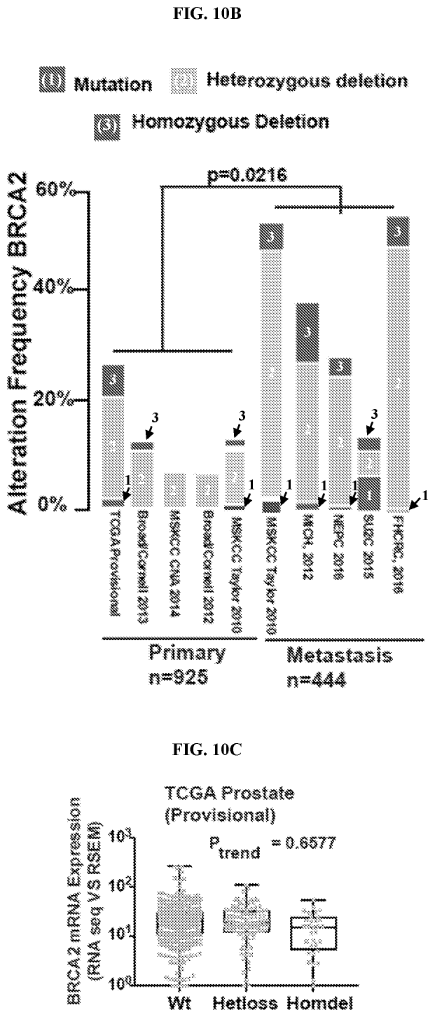

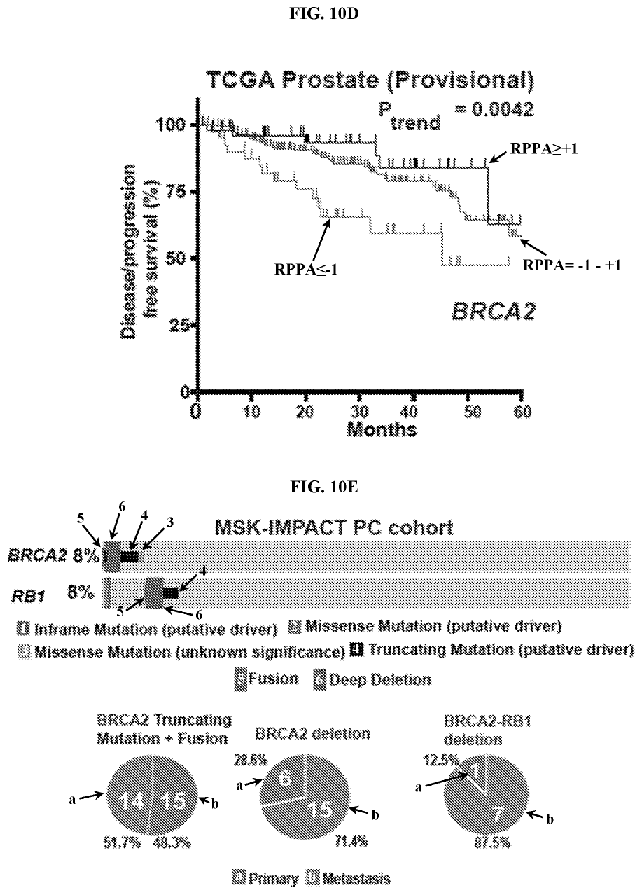

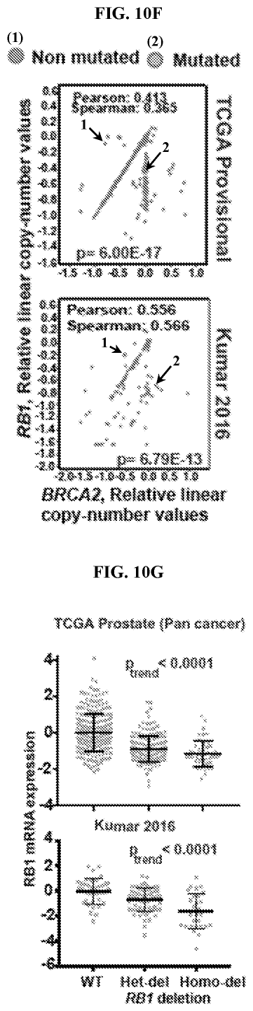

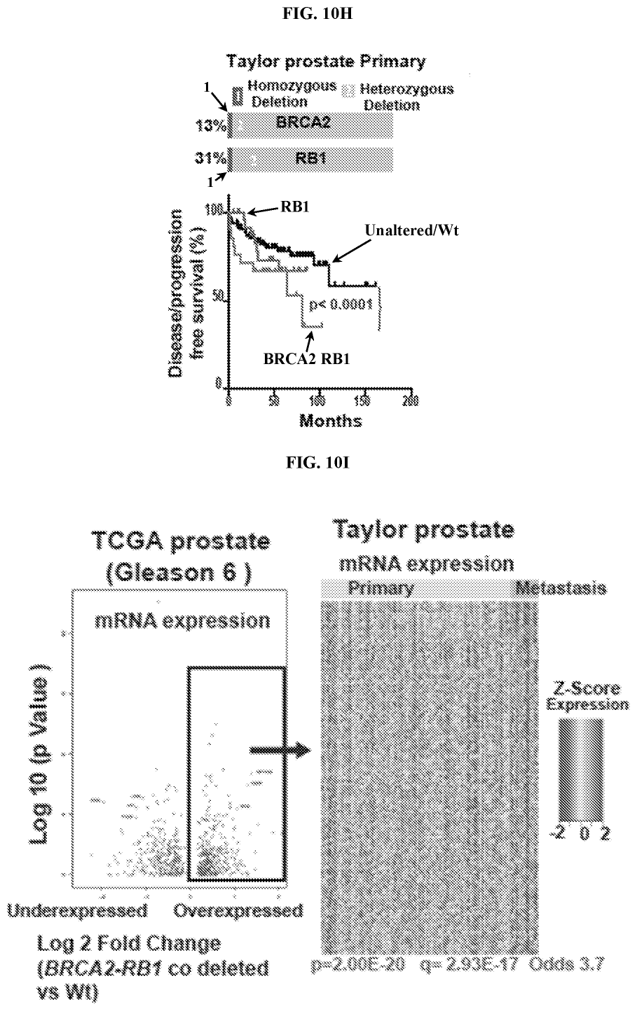

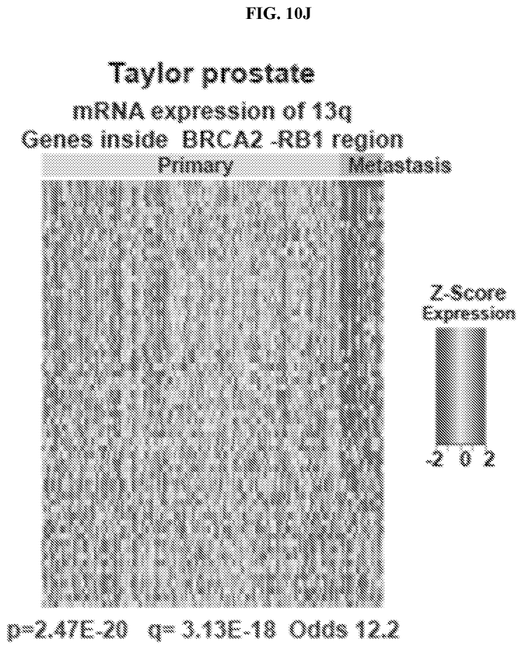

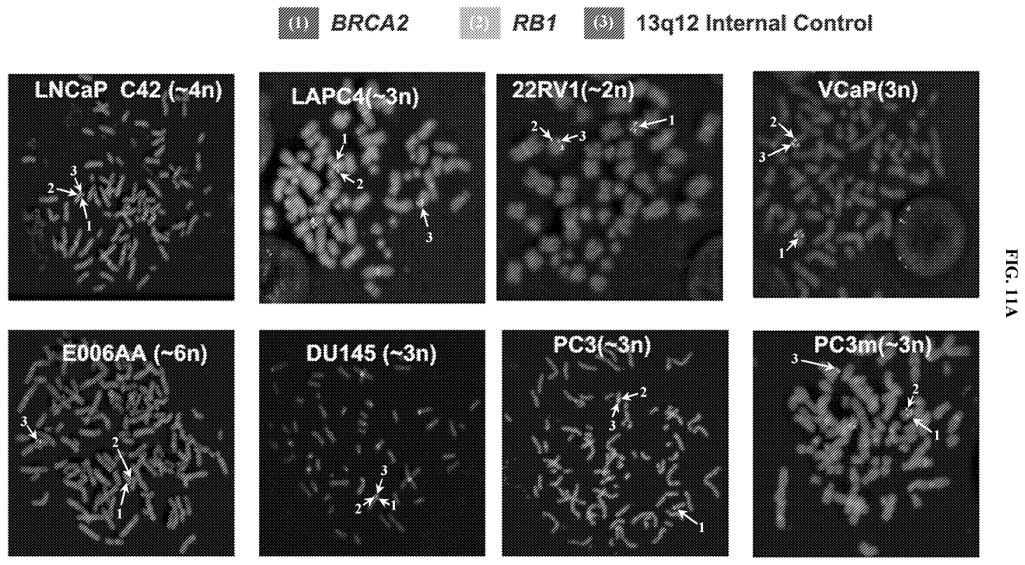

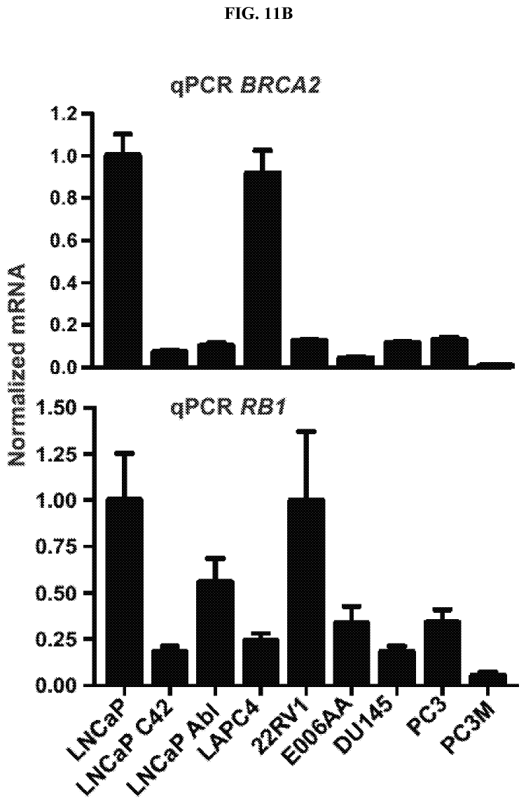

BRCA2 status was analyzed in a pan-cancer dataset derived from cBioPortal for Cancer Genomics, where BRCA2 is frequently altered (BRCA2 alteration frequency >5% of cases; number of cases >50). As shown in FIG. 10A, more frequent homozygous deletions of BRCA2 were observed in prostate cancer (localized and mCRPC) than in other cancers (whereas other cancers exhibited frequent mutational events). In the Armenia et al. prostate cancer dataset, which contains both primary (localized) and mCRPC cases (Armenia et al. Nat Genet. 50(5):645-51 (2018)), BRCA2 alterations were observed in ˜10% of mCRPC cases compared to only ˜2.5% in primary cases; P=2.91e-06; (data not shown). BRCA2 alterations were more common than other major DDR pathway components, and were enriched in mCRPC relative to localized disease, suggesting it is associated with, if not a driver of, aggressive disease (FIG. 4A and data not shown). Note that the Armenia cohort was not designed to determine germline mutations of DDR pathway components.

Further in-depth analysis of the BRCA2 status in multiple independent publicly available and published prostate cancer datasets (from cBioPortal) revealed that a significant fraction of localized as well as metastatic cases exhibit deletion (homozygous and heterozygous) of BRCA2, which had not been previously described (FIG. 10B). This analysis also revealed that BRCA2 alterations (homozygous or heterozygous deletions, as well as mutations, denoted as BRCA2 alterations throughout this study) were significantly enriched (P=0.0216) in this combined mCRPC dataset (n=444) compared to a primary (localized) dataset (n=925) (FIG. 10B). While the TCGA provisional cohort was not designed to look at clinical outcomes (overall survival), in the available data, BRCA2 deletion is significantly associated with shorter disease/progression-free survival (5 years; Ptrend=0.0059), as shown in FIG. 4B. Interestingly, any difference in disease progression could not be detected between patients with homozygous and heterozygous BRCA2 deletions (FIG. 4B). These observations suggests that even heterozygous loss of BRCA2 may be associated with a more aggressive form of prostate cancer.

As shown in FIG. 4C, homozygous and even heterozygous deletion of BRCA2 significantly reduced BRCA2 protein levels as determined by reverse phase protein array (RPPA) (Ptrend=0.0083). Any difference in BRCA2 protein expression between heterozygous and homozygous cases could not be detected (FIG. 4C). However, in the same TCGA prostate cancer cohort, a relationship between BRCA2 deletion (either homozygous or heterozygous) and BRCA2 mRNA expression was not detected (FIG. 10C). Heterozygous deletion of BRCA2 is sufficient to reduce protein level but not mRNA level, indicating that single copy loss may lead to haploinsufficiency of BRCA2 protein expression. As shown in FIG. 10D, decreased BRCA2 protein expression is significantly correlated with shorter disease-free survival. Taken together, for the first time it was demonstrated the potential clinical significance of heterozygous deletion of BRCA2 in primary prostate cancer through loss of BRCA2 protein expression.

These results demonstrate that homozygous or heterozygous deletion of BRCA2 plays a significant role in more aggressive form of prostate cancer. These results also suggest that more aggressive form of prostate cancer harboring homozygous or heterozygous deletion of BRCA2 are sensitive to the PARP inhibitors of the present technology. Accordingly, the methods disclosed herein are useful for selecting a prostate cancer patient for treatment with a PARP inhibitor, and/or treating or preventing metastatic castration-resistant prostate cancer in a patient in need thereof.

### Example 6: BRCA2 is Frequently Co-Deleted with RB1 in Aggressive Prostate Cancer

A prior sequencing study revealed that co-deletion (heterozygous and homozygous) of RB1 and BRCA2 is present in a significant fraction of primary prostate cancers (˜ 25% in TCGA provisional cohort (FIG. 4D, top panel). Cancer Genome Atlas Research Network. Cell 163(4):1011-25 (2015). Interestingly, in the MSK-IMPACT prostate cancer cohort (36), BRCA2 homozygous deletion, not mutation, was observed to be enriched in metastatic cases and co-occurs with homozygous RB1 deletion (FIG. 10E). In the TCGA and Taylor prostate cancer datasets, patients with primary prostate cancer who have BRCA2-RB1 co-deletion have significantly shorter disease/progression-free survival compared to patients with deletion of neither or of RB1 alone (FIGS. 4D (bottom panel) and 10H (bottom panel), while deletion of BRCA2 without RB1 is rare (FIGS. 4D (top panel) and 10H (top panel). Also, as shown in FIG. 10F, BRCA2 copy number and RB1 copy number are correlated in both primary prostate cancer (TCGA) and mCRPC (Kumar) cohorts. However, note that unlike BRCA2, RB1 mRNA expression was significantly associated with RB1 genomic deletion (heterozygous and homozygous) in primary (TCGA) and mCRPC (Kumar) cohorts, as shown in FIG. 10G.

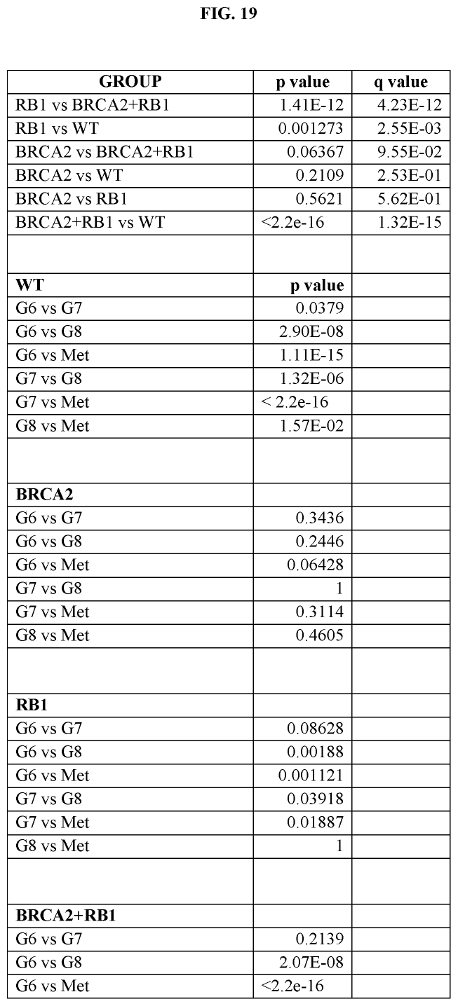

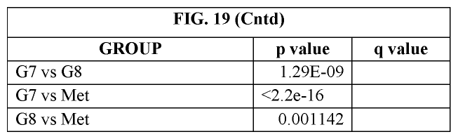

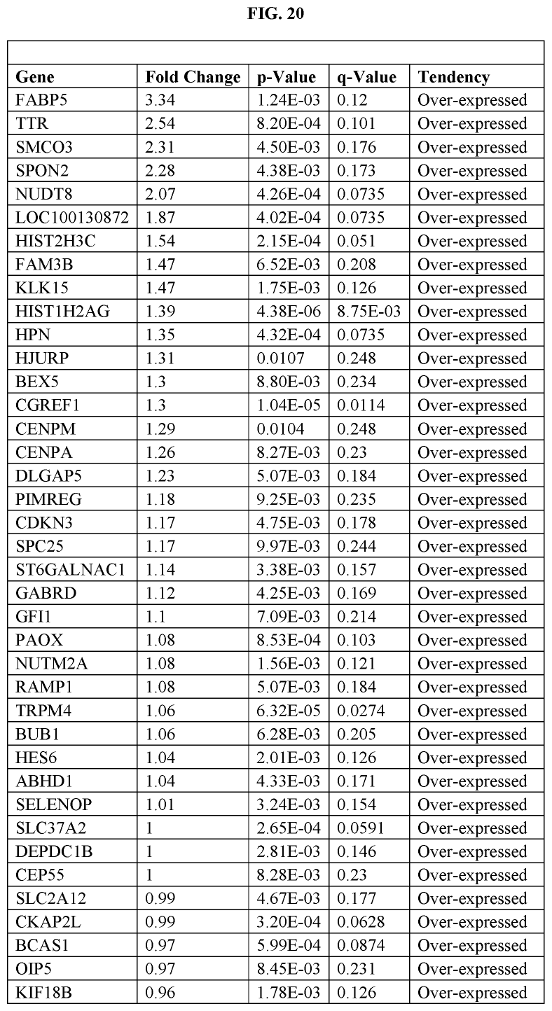

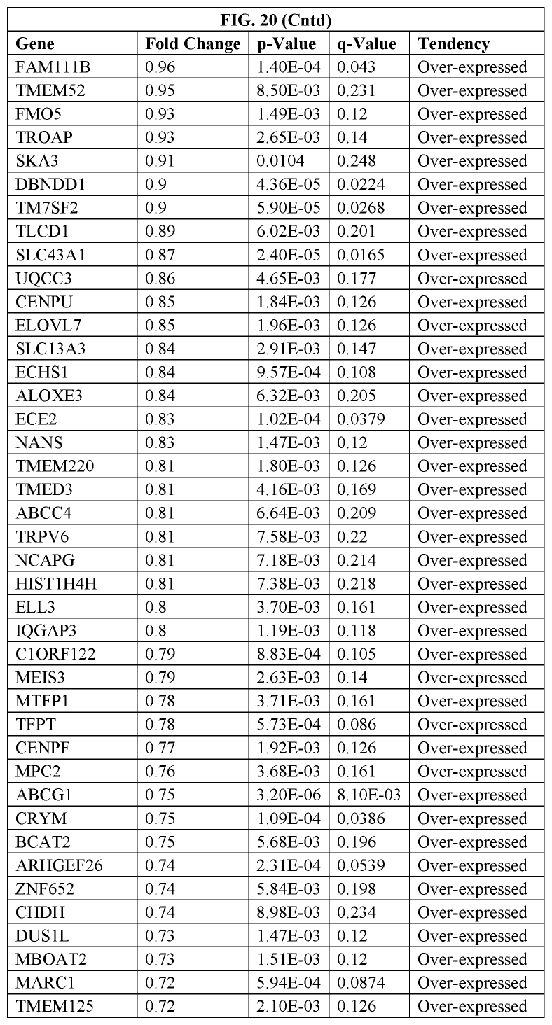

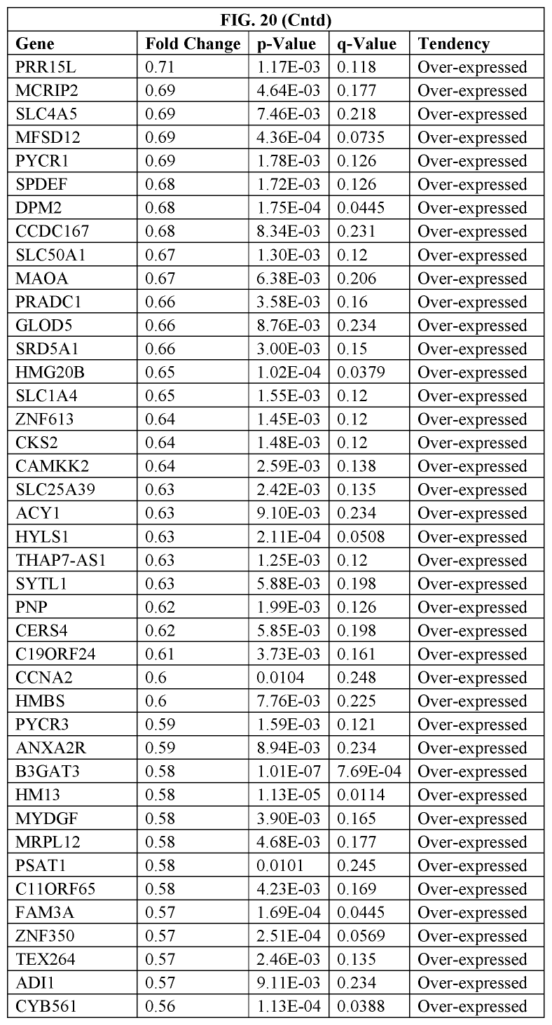

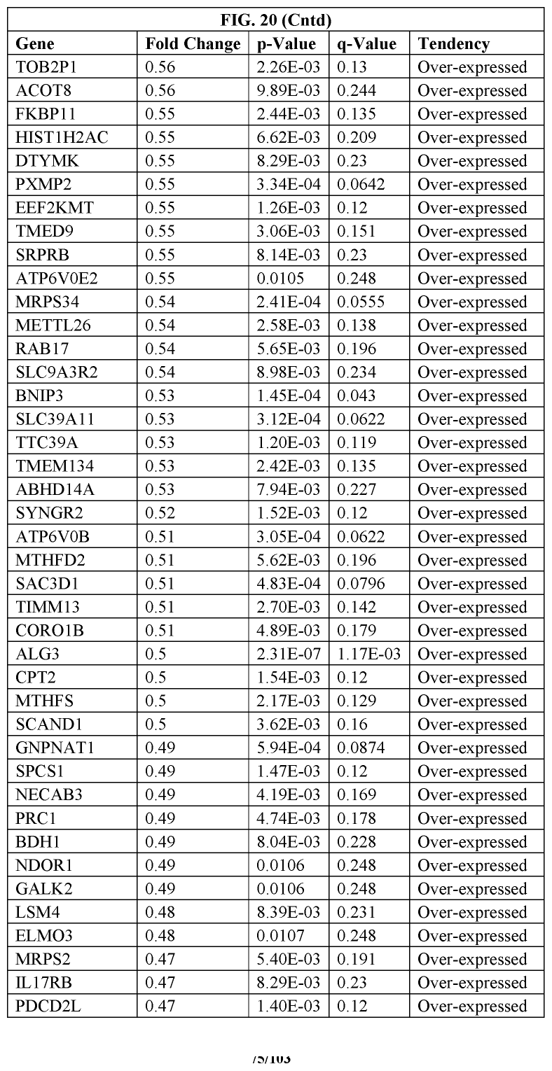

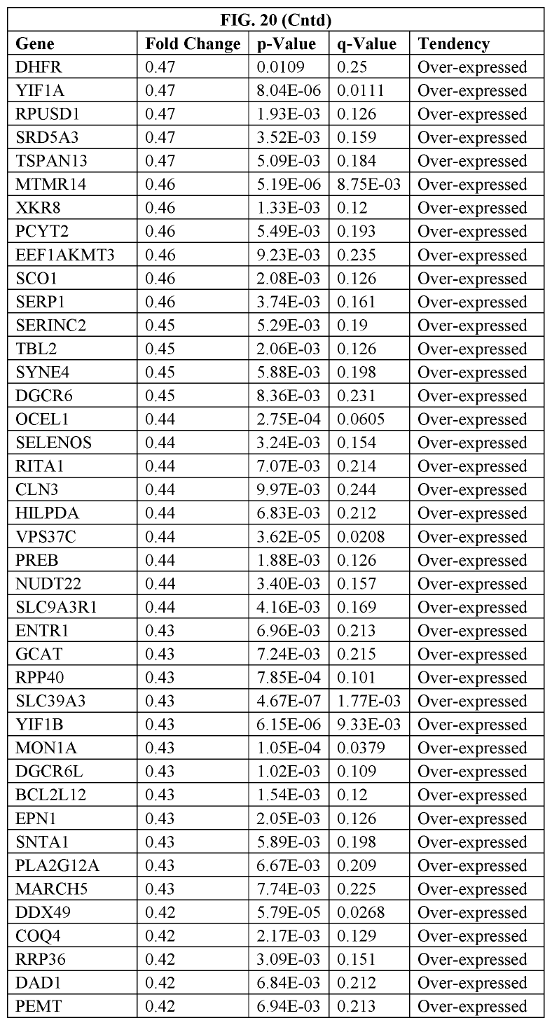

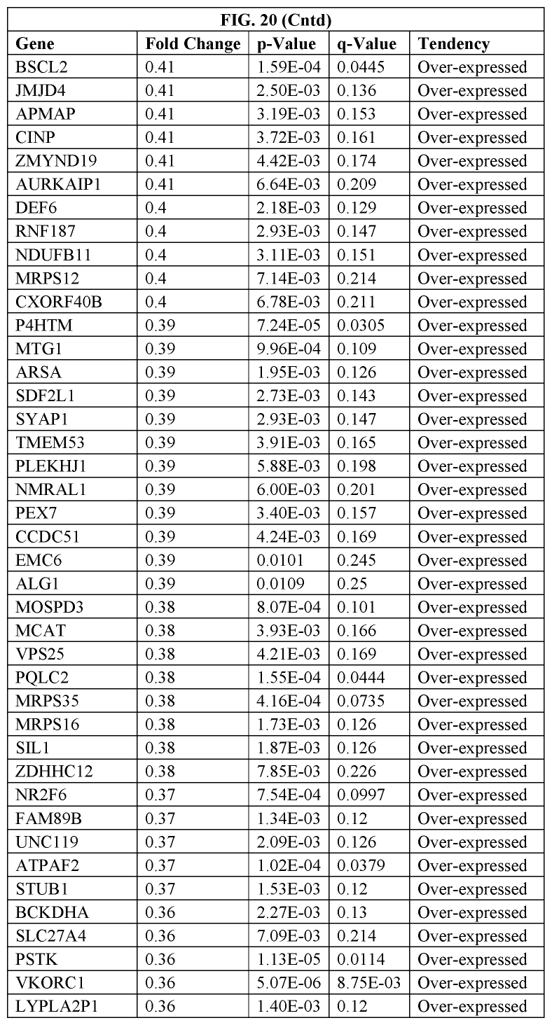

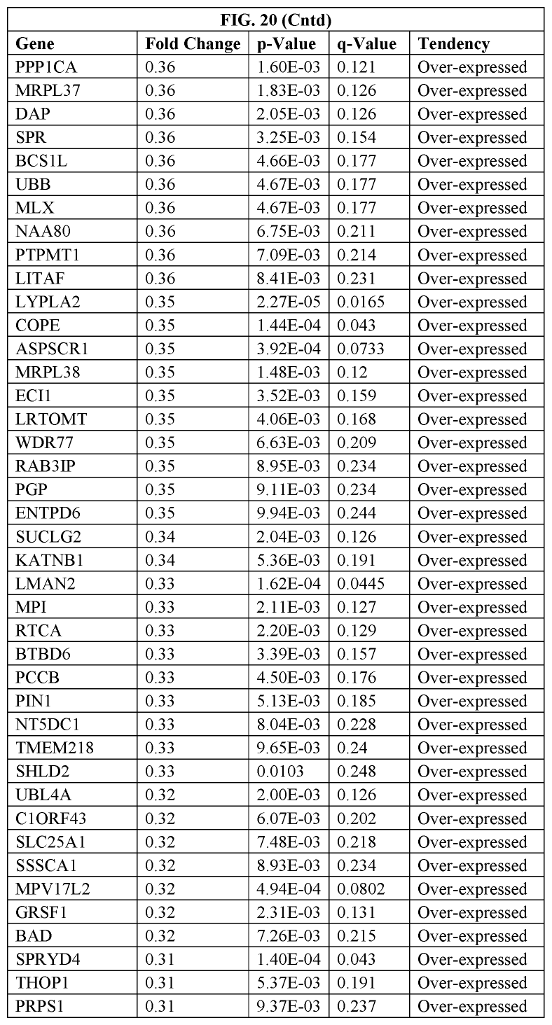

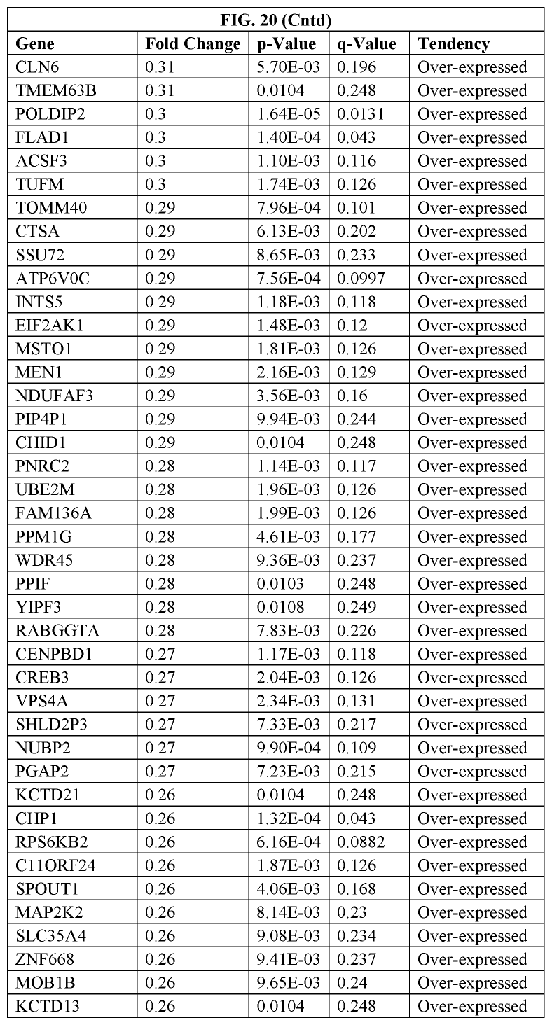

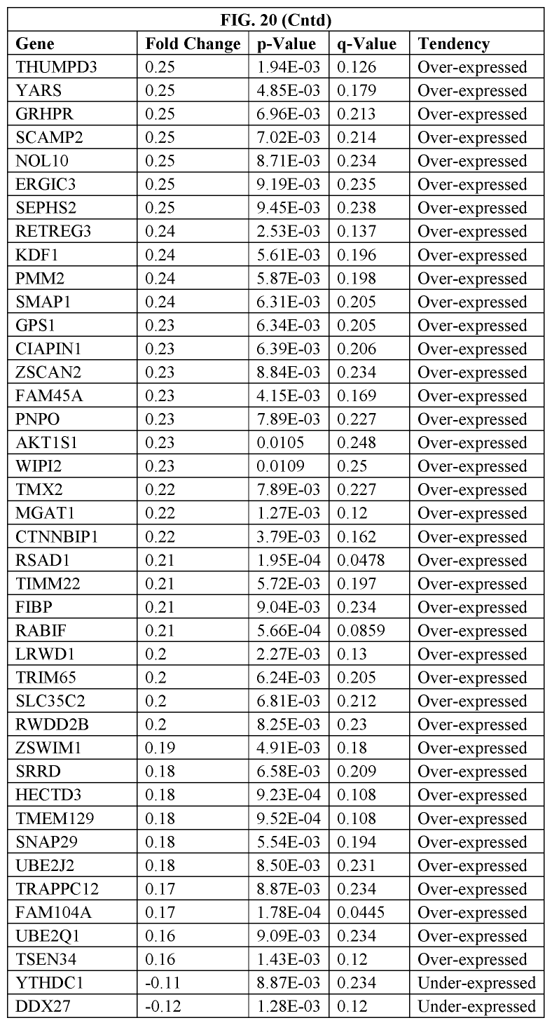

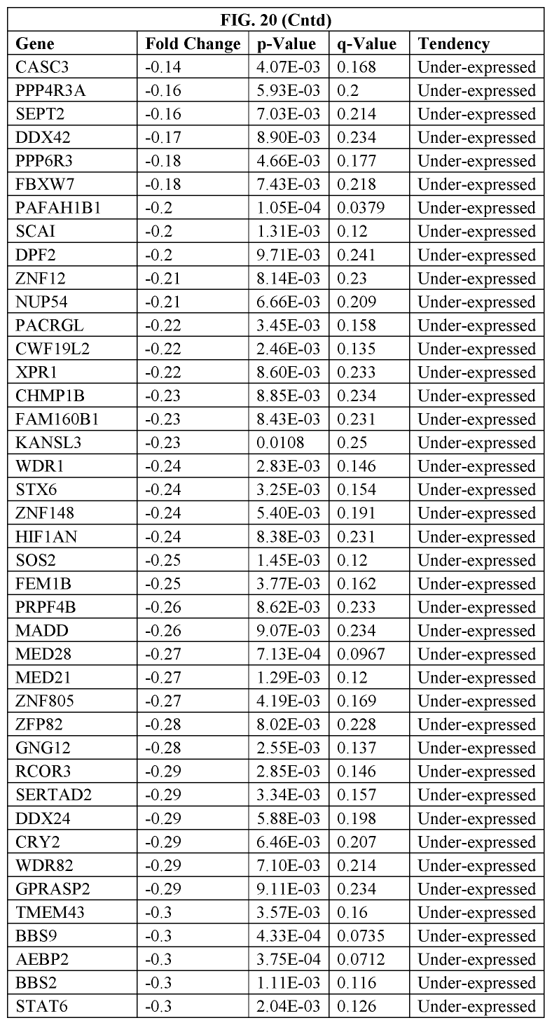

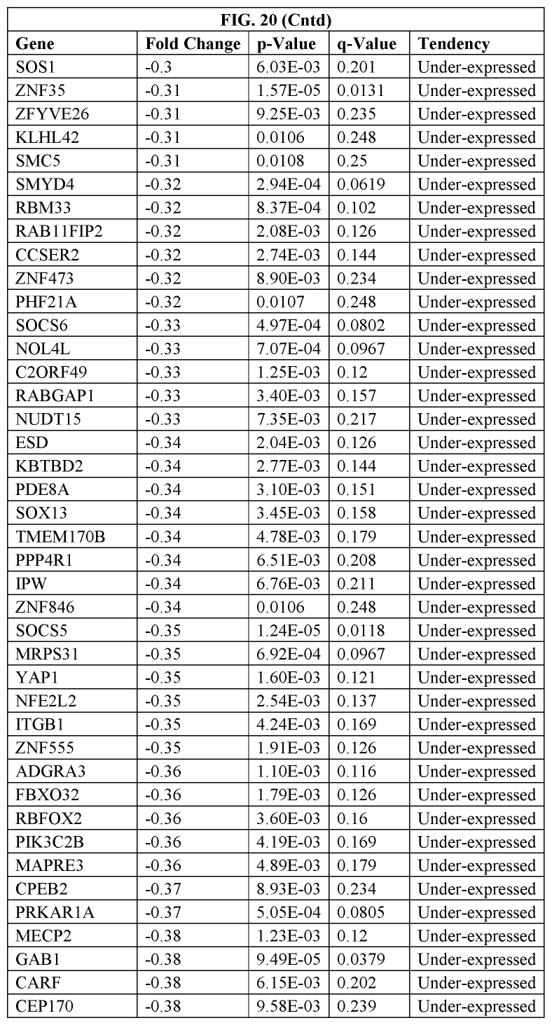

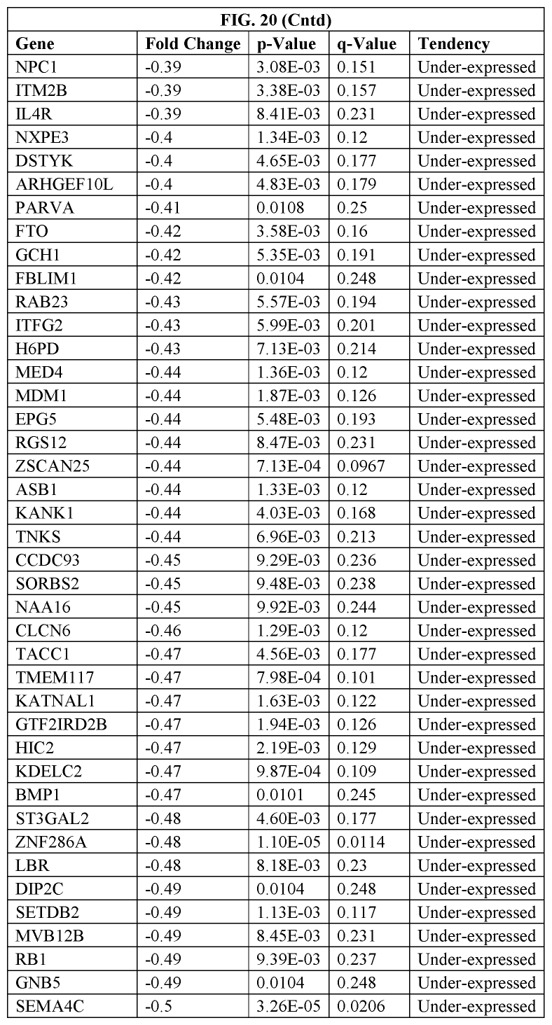

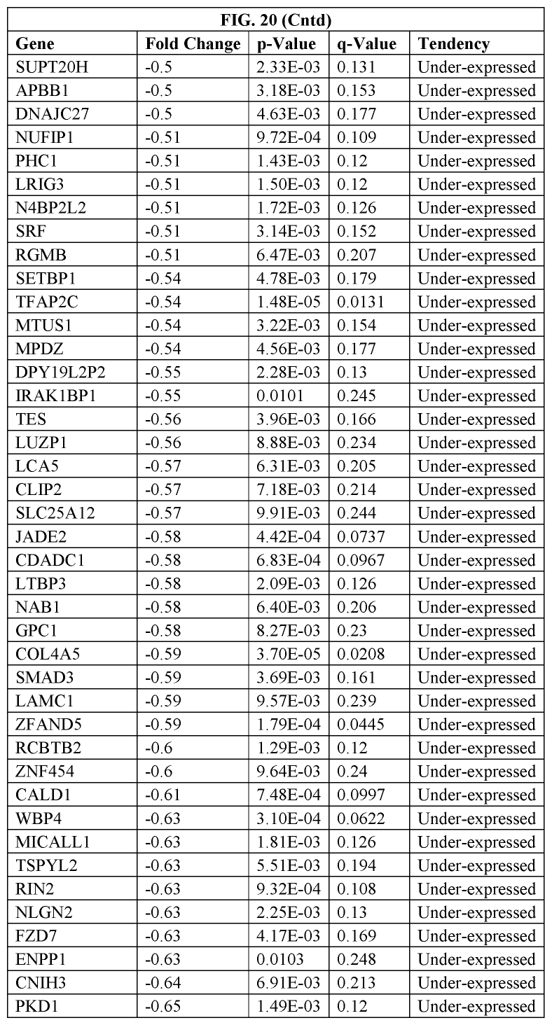

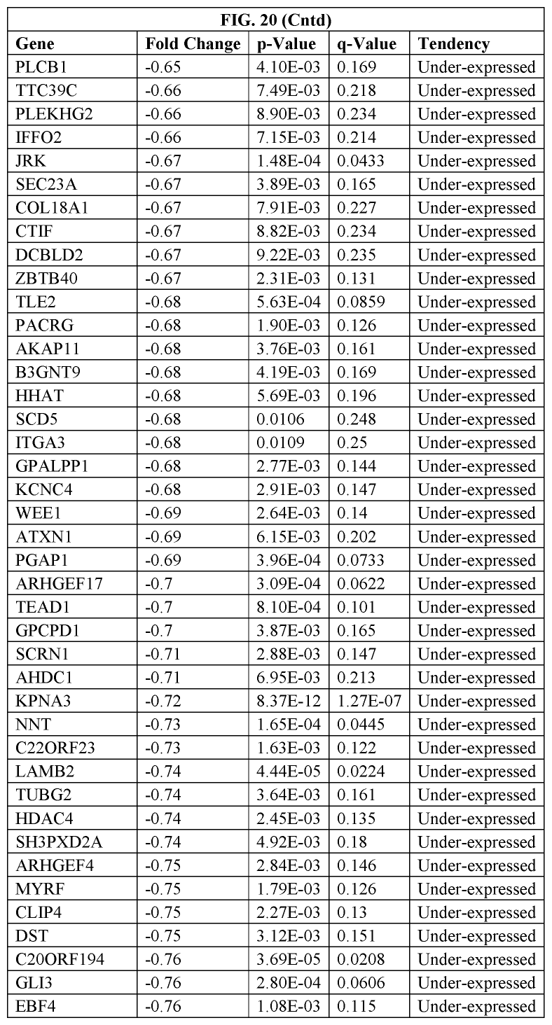

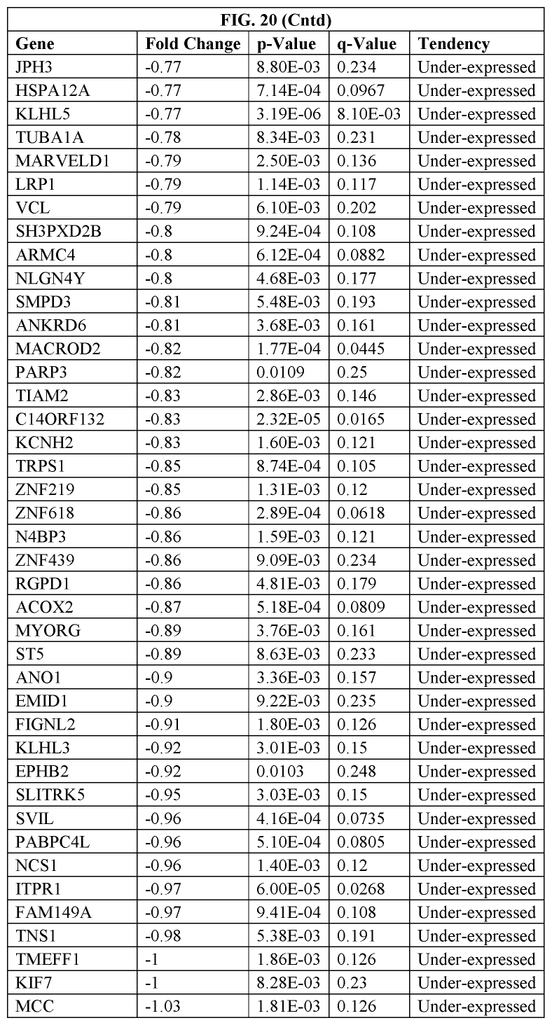

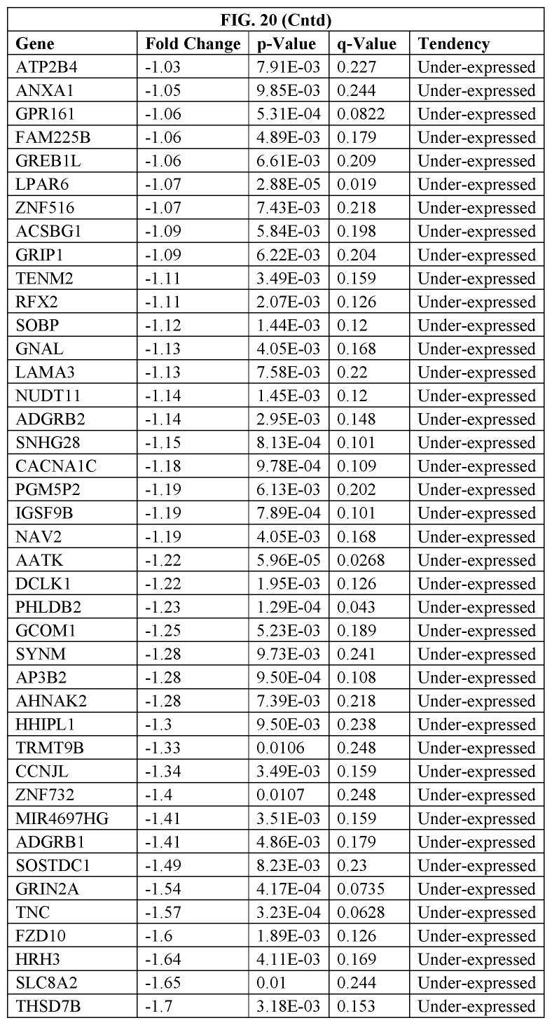

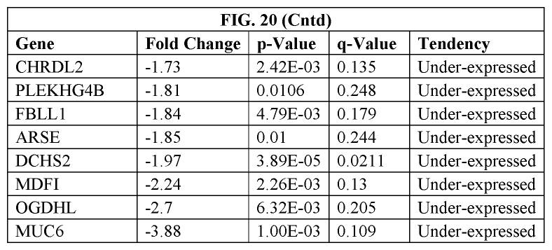

Co-deletion of BRCA2-RB1 was significantly enriched in high Gleason grade prostate cancer as well as in metastases (FIGS. 4E and 19). However, as shown in FIG. 4E, deletion of RB1 alone is not significantly associated with stage or progression to metastasis. The details of the co-deletion and P-values of each stage are summarized in FIG. 19. It was also observed that ˜10% of low-grade (Gleason 6) cases harbor genomic co-deletion of BRCA2 and RB1 (FIG. 10I). The mRNA expression of the genes that are upregulated due to co-deletion of BRCA2 and RB1 in Gleason 6 disease in TCGA provisional prostate cancer cohort was established (FIGS. 10I and 20). To further assess the importance of the BRCA2-RB1 co-deletion in low grade primary prostate cancer, the BRCA2-RB1 loss Gleason 6 gene signature from TCGA was compared to the metastatic prostate cancer signature using Oncomine suite. Rhodes et al., Neoplasia. 6(1):1-6 (2004). In the Taylor cohort, was observed enrichment of this BRCA2-RB1 loss Gleason 6 gene signature in metastatic prostate cancer (p=2.00E-20, odds 3.7), as shown in FIG. 10I.

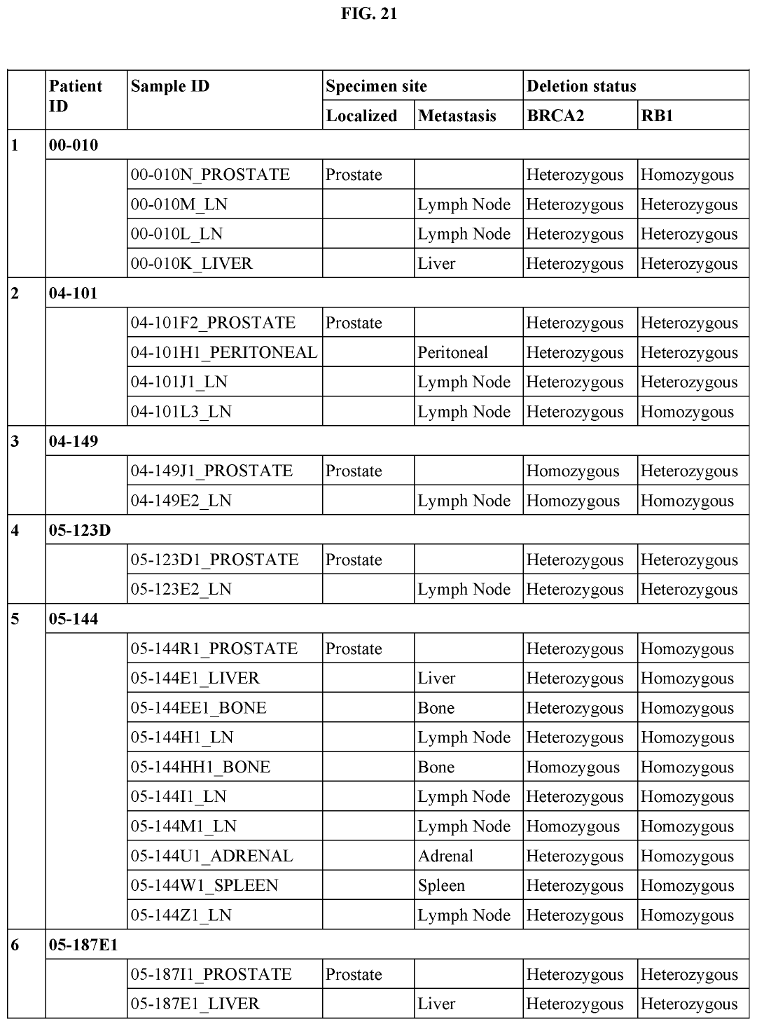

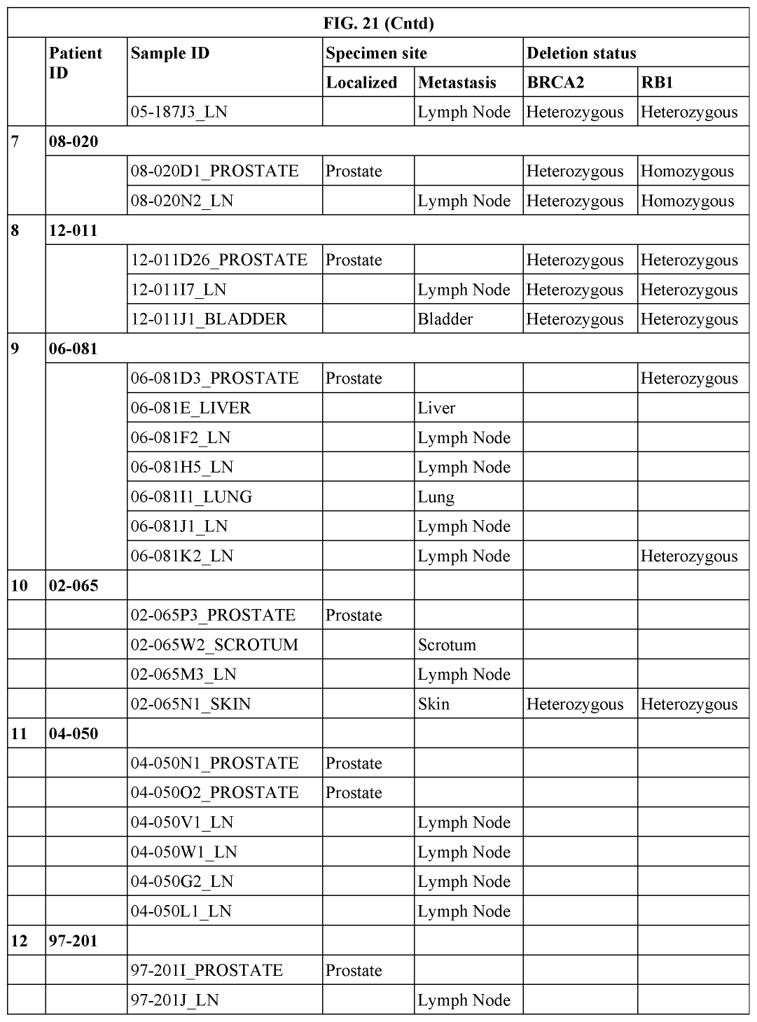

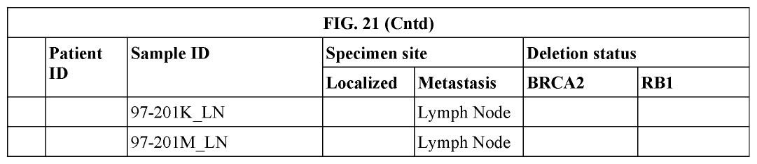

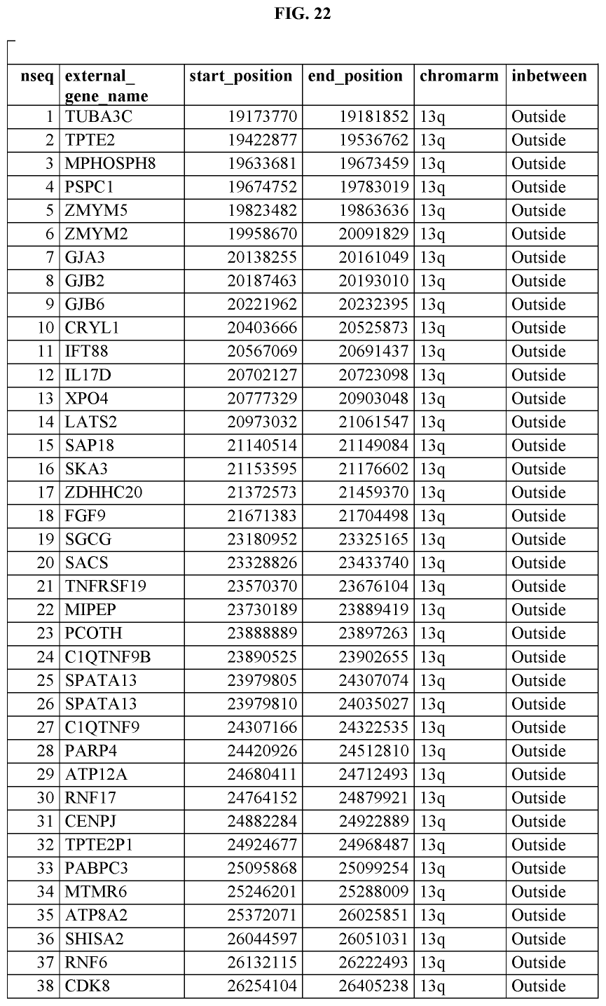

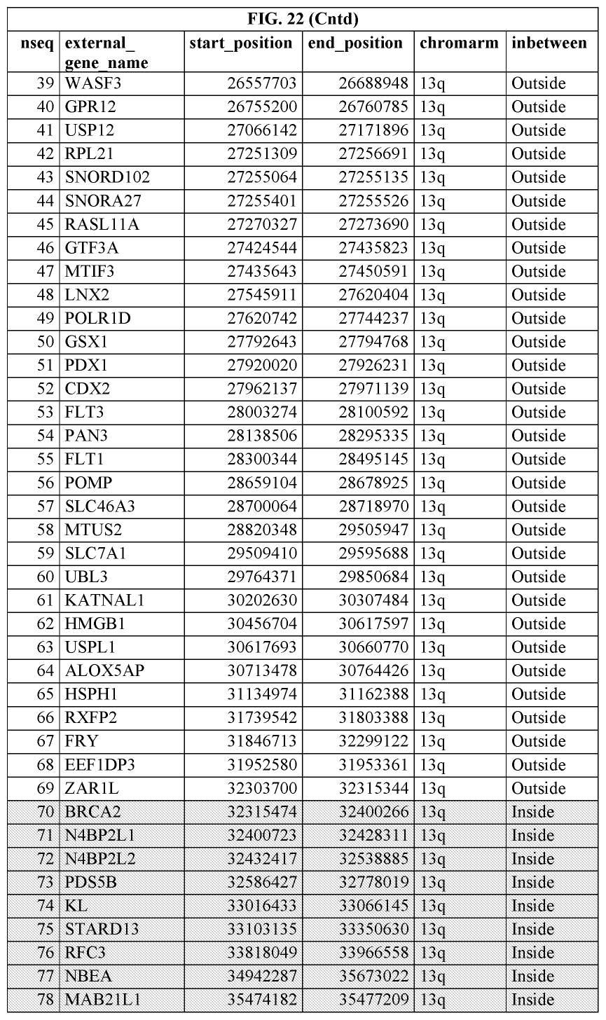

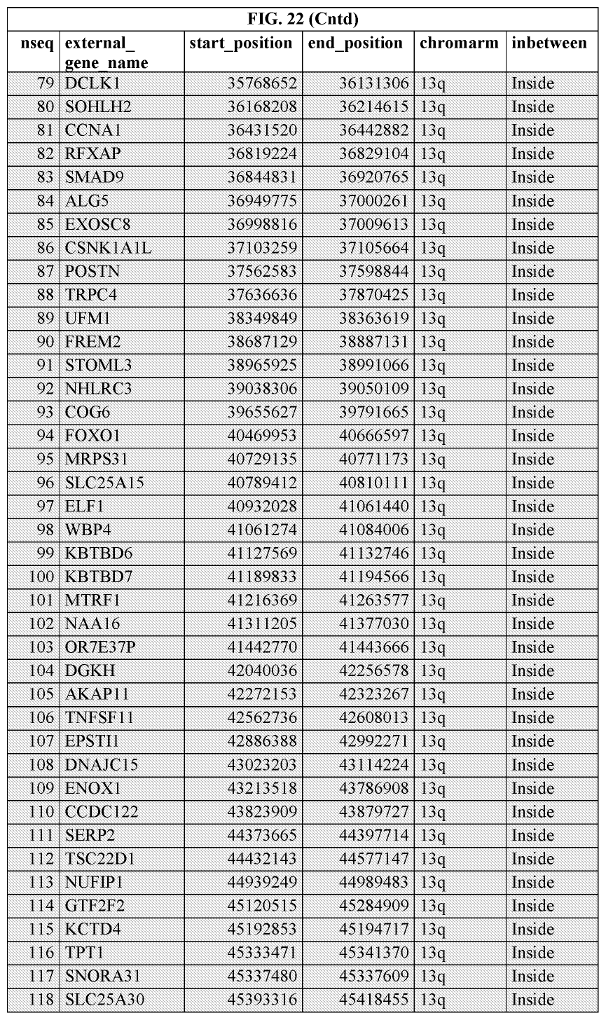

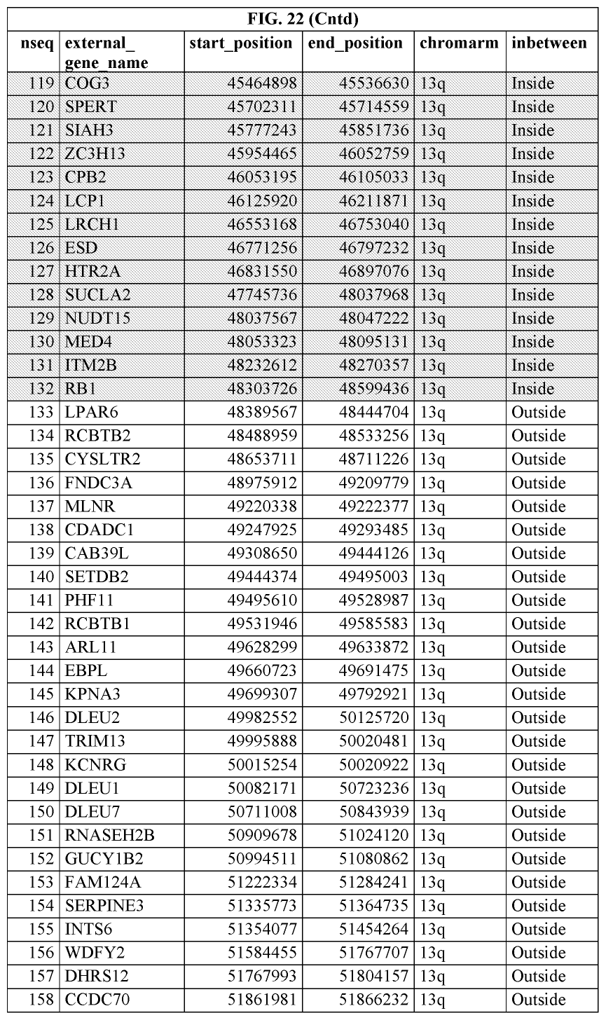

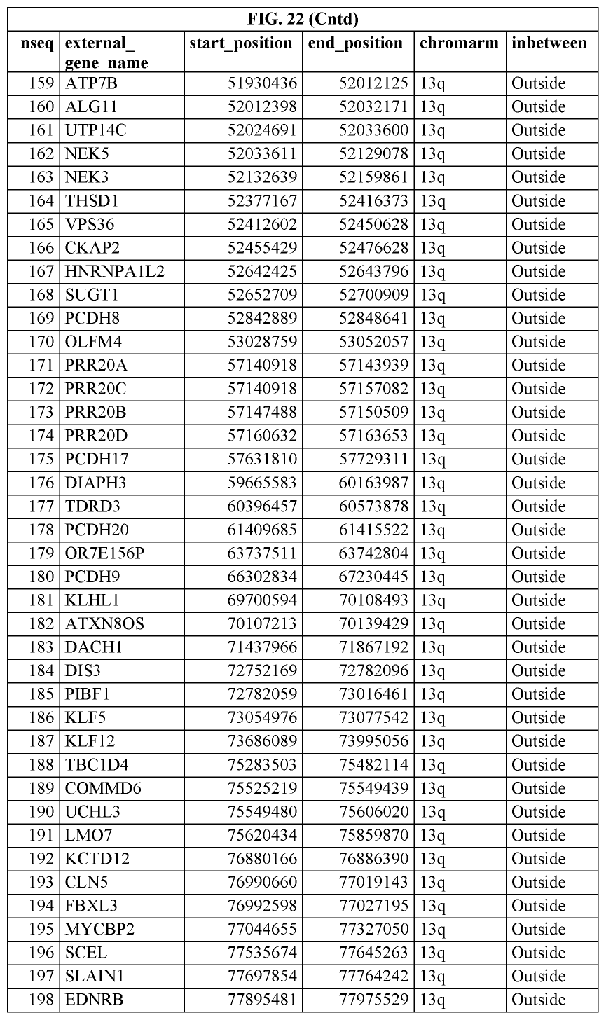

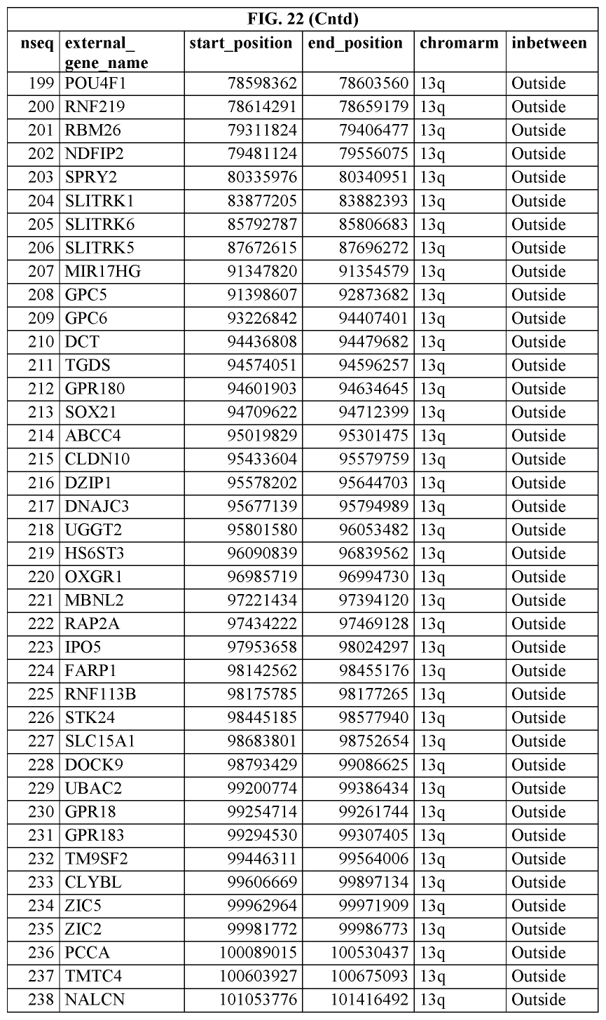

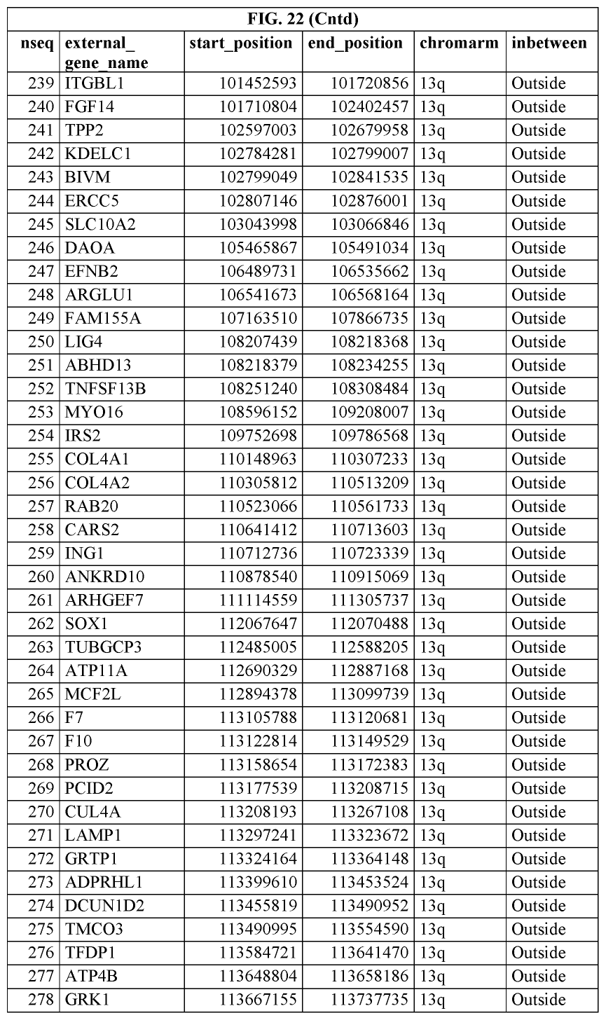

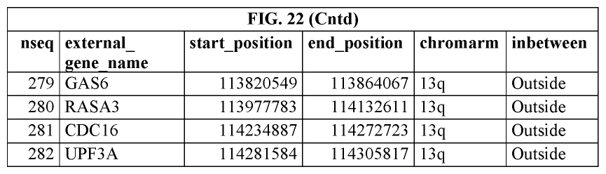

This study was extended to match (localized and metastatic) prostate cancer samples in the Kumar et al. cohort to further assess the direct association between co-deletion of both genes and metastatic progression. FIG. 21 displays the 12 mCRPC patients in the Kumar et al. cohort that had matched localized and metastatic samples. As shown in FIG. 21, all 8 patients (66.7%) who had co-deletion of BRCA2 and RB1 in their localized tumors retained their BRCA2-RB1 co-deletion in all of their metastatic tumors, indicating that this co-deletion may be critical to metastatic progression. Interestingly, for the one patient (06-081) who had an RB1 deletion alone in his localized prostate tumor, the RB1 deletion was not seen in all his metastatic tumors. These data suggest that co-deletion of BRCA2 and RB1 in primary disease is likely a driver to mCRPC.

In an analysis of the Armenia et al. dataset, which contains both primary and mCRPC cases, it was found that BRCA2-RB1 co-loss in early prostate cancer appeared to be significantly associated with increased fraction of genome altered, as shown in FIG. 4F. Fraction of genome altered is a biomarker associated with genomic instability and also appeared to be associated with prostate tumor aggressiveness, suggesting that BRCA2-RB1-null tumors are likely aggressive in nature.

These results demonstrate that BRCA2-RB1 co-loss in prostate cancer is likely a driver to metastatic castration-resistant prostate cancer (mCRPC). Accordingly, the methods disclosed herein are useful for selecting a prostate cancer patient for treatment with a PARP inhibitor, and/or treating or preventing metastatic castration-resistant prostate cancer in a patient in need thereof.

### Example 7: Deletion of BRCA2-RB1 Region of Chromosome 13q in Prostate Cancer

As shown in FIG. 4G, copy number segment analysis of primary and mCRPC samples from the Armenia et al. dataset indicated frequent deletion of the BRCA2-RB1 region of chromosome 13q. Copy number loss of other genes located in the BRCA2-RB1 region was also observed in patients who harbored the co-deletion of BRCA2 and RB1 (FIG. 4H, top panel). To further assess the nature of this deletion the mRNA of all the protein coding genes on chromosome 13q was analyzed (FIGS. 4H (bottom panel) and 22). As shown in FIG. 4H, the mRNA expression of chromosome 13q genes between BRCA2 and RB1 was lower in BRCA2-RB1 deleted patients compared to wild-type patients in the TCGA 2015 cohort. More in-depth analysis in the TCGA pan-cancer prostate cohort (extended TCGA 2015 cohort) showed that the mRNA expression of genes located downstream of BRCA2 was significantly lower than for genes located upstream of BRCA2 in patients who harbored a co-deletion of BRCA2 and RB1 (FIG. 4I). These data indicate an interstitial deletion of the BRCA2-RB1 region in prostate cancer rather than deletion of the entire chromosome 13q arm.

An association between the loss of mRNA expression of BRCA2-RB1 region genes in the mCRPC cohorts compared to primary (localized) prostate cancer was observed. Loss of expression of these genes was seen (to a greater degree) in mCRPC compared to primary cases in the Grasso (p=2.12E-6, OR 4.4) and Taylor (p=2.47E-20, OR 12.2) cohorts (Grasso: primary n=59, mCRPC n=35; Taylor primary n=131, mCRPC n=19; FIGS. 4J and 10J). Note that in the Grasso cohort, the mCRPC specimens were isolated by rapid autopsy from metastatic sites. Grasso et al., Nature. 487(7406):239-43 (2012).

Taken together, these data suggest that an interstitial deletion of the BRCA2-RB1 region of chromosome 13q may be associated with castration resistance and metastasis. Accordingly, the methods disclosed herein are useful for selecting a prostate cancer patient for treatment with a PARP inhibitor, and/or treating or preventing metastatic castration-resistant prostate cancer in a patient in need thereof.

### Example 8: Castration-Resistant Aggressive Human Prostate Cancer Cells Exhibit Genomic Co-Deletion of BRCA2 and RB1

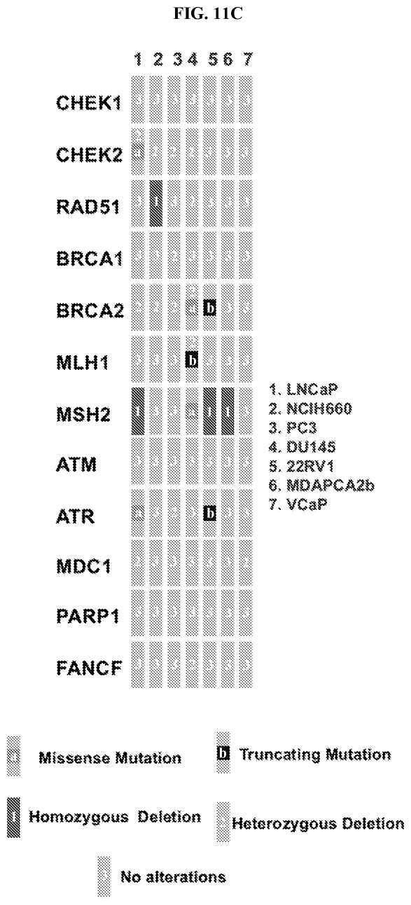

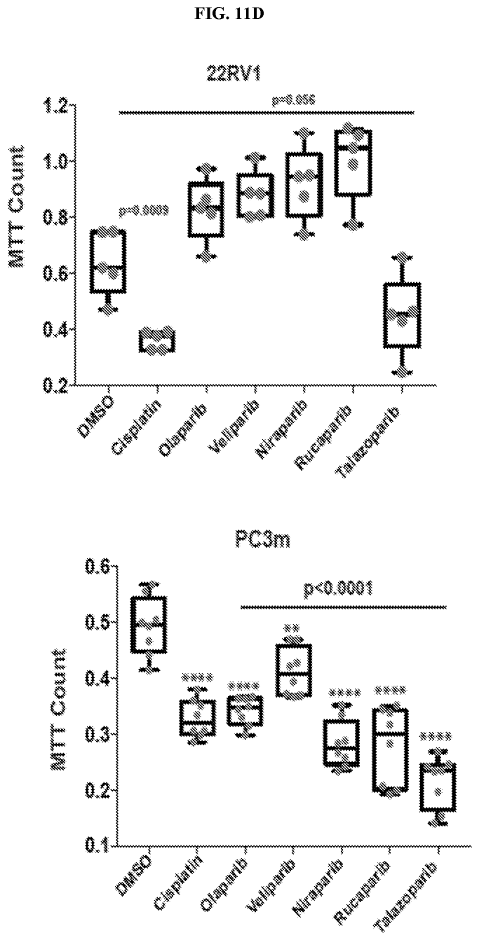

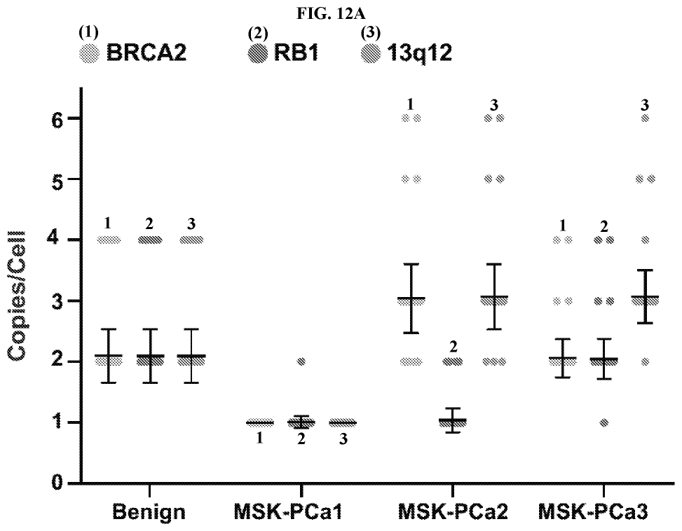

To further confirm that in prostate cancer BRCA2 is frequently deleted with RB1 rather than alone, a 3-color FISH probe was developed to apply to human cells. The probes were validated on human peripheral blood and immortalized prostate cells (RWPE-1), in which almost every cell exhibits 2 copies of BRCA2 and RB1 (FIGS. 5A, 5C, 11A, and data not shown). As shown in FIGS. 5A and 11A, human CRPC cell lines E006AA, DU145, PC3, and PC3M exhibited uniform heterozygous co-deletion of BRCA2 and RB1 (additional data not shown). Heterozygous co-deletion of BRCA2 and RB1 is associated with high fraction of genome altered in PC3 and DU145 cells but not in 22RV1 and MDA PC2B cells (absence of co-deletion) or in LNCaP cells (partial co-deletion) in The Cancer Cell Line Encyclopedia, as shown in FIG. 5B. Barretina et al., Nature. 483(7391):603-7 (2012). The detailed analysis of the BRCA2-RB1 copy number and ploidy of individual prostate cancer cell lines was also performed (data not shown). Most importantly, heterozygous co-deletion of BRCA2 and RB1 was detected in VCaP cells (not noted in sequencing study), which also display a high fraction of genome altered (FIGS. 5A, 5B and 11A).

As shown in FIGS. 5A-5C, ˜60% of parental LNCaP cells were found to harbor loss of one or more copies of RB1, including ˜10% with co-deletion of BRCA2 (additional data not shown). Heterogeneity in chromosome number (ploidy, 2-10 copies of chromosome/cell) was observed in LNCaP cells indicating the heterogeneous nature of the parental LNCaP cell line (FIG. 5D and data not shown). Previous studies have identified a castration-resistant low-PSA subpopulation among parental LNCaP cells. Qin et al., Cell Stem Cell 10(5):556-69 (2012). This is consistent with the current observation and suggests the clonal expansion of a subpopulation of LNCaP cells in the castrate environment as demonstrated previously. Qin et al., Cell Stem Cell 10(5):556-69 (2012). Interestingly, the LNCaP-derived hormone-independent LNCaP-Abl cell line (able to grow in androgen-independent culture condition) exhibits uniform co-loss of 1 of 4 copies of BRCA2 and RB1, further indicating this co-deletion is directly associated with ADT resistance and also may indicate a clonal expansion of castration-resistant BRCA2-RB1-deleted population from parental LNCaP cells, as shown in FIGS. 5A, 5C, and 5D (additional data not shown).

As shown in FIGS. 5E and 11B, the protein and mRNAs of both genes were consistently decreased in these cell lines. Although the castration-resistant LNCaP subclone C42 exhibited uniform heterozygous deletion of RB1 only, attenuation of BRCA2 protein and mRNA was observed as well. This indicates that an additional mechanism of loss of BRCA2 in RB1-deleted cells may lead to the castration-resistant phenotype (FIGS. 5A, 5E and 11B).

The immunoblot analysis showed that the human CRPC cell lines DU145, PC3, and the PC3 derivative PC3M which exhibited uniform heterozygous co-deletion of BRCA2 and RB1 as shown in FIGS. 5A and 11A, and also exhibited the EMT-like phenotype, including upregulation of vimentin and loss of E-cadherin expression (FIG. 5F). However, LAPC4, 22RV1 (mutant BRCA2 but wild-type RB1), and LNCaP (RB1 partial deletion but wild-type BRCA2) exhibited more epithelial-like characteristics as shown in FIG. 5F. Co-deletion of BRCA2-RB1 in LNCaP-Abl cells was also associated with upregulation of vimentin protein expression, which is consistent with the current observations (FIG. 5F).

As shown in FIG. 5G, co-deletion of BRCA2-RB1 in the LNCaP-Abl cell line was consistently associated with sensitivity to various PARPi (rucaparib and talazoparib) and platinum drugs compared to parental LNCaP cells. Note that although parental LNCaP cells harbor several defects in various DDR genes (FIG. 11C), the LNCaP subline LNCaP-Abl exhibited more PARPi-mediated cell growth inhibition compared to parental LNCaP cells (FIG. 5G). Although the COSMIC cancer cell line dataset showed that LNCaP cells harbor a deletion-frameshift mutation of BRCA2 (p.D946fs*14), sequencing studies from The Cancer Cell Line Encyclopedia (FIG. 5B) and the Taylor prostate dataset were unable to detect such BRCA2 mutation in parental LNCaP cells. A prior study also showed that LNCaP cells express a wild-type BRCA2 transcript. These data suggest that heterozygous co-deletion of BRCA2 and RB1 in LNCaP-Abl cells is sufficient to reduce the mRNA expression of both genes and therefore induce sensitivity to PARPi (FIG. 5G). Similarly, PC3M cells, which also harbor genomic co-deletion of BRCA2 and RB1, show sensitivity to various PARPi or platinum drugs (FIG. 11D, bottom). In contrast, it was observed that the 22RV1 cell line, which harbors a T3033Nfs*11 mutation in BRCA2, showed sensitivity to cisplatin and modest sensitivity to talazoparib but not to other PARPi (FIG. 11D, top panel).

Taken together, these results indicate that co-loss of BRCA2-RB1 is a cell line-independent event and is frequently associated with castration resistance and leads to heightened sensitivity to PARP inhibitors. Accordingly, the methods disclosed herein are useful for selecting a prostate cancer patient for treatment with a PARP inhibitor, and/or treating or preventing metastatic castration-resistant prostate cancer in a patient in need thereof.

### Example 9: Organoids Derived from Human mCRPC Patients Harbor Co-Heterozygous Deletion of BRCA2 and RB1

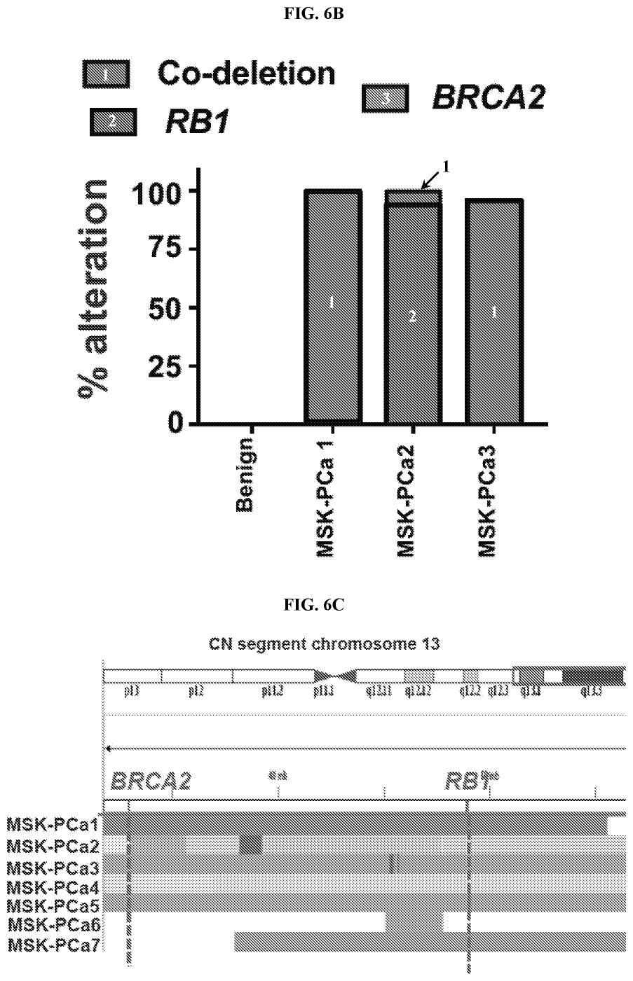

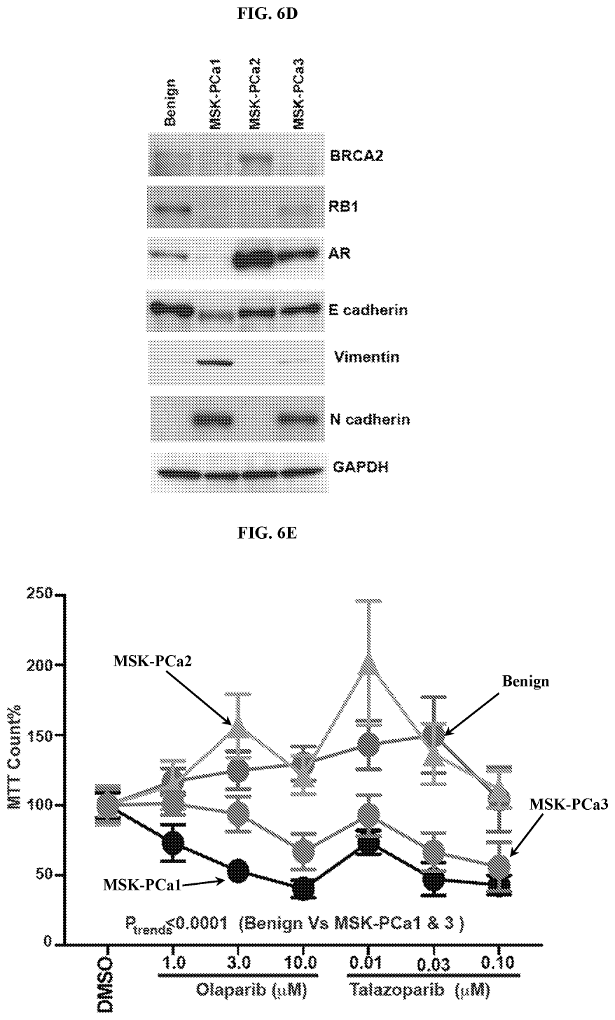

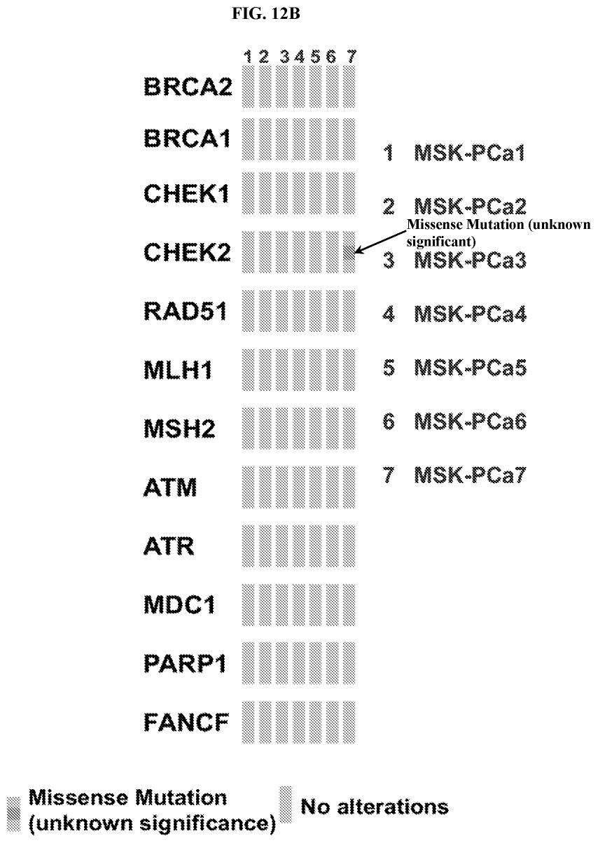

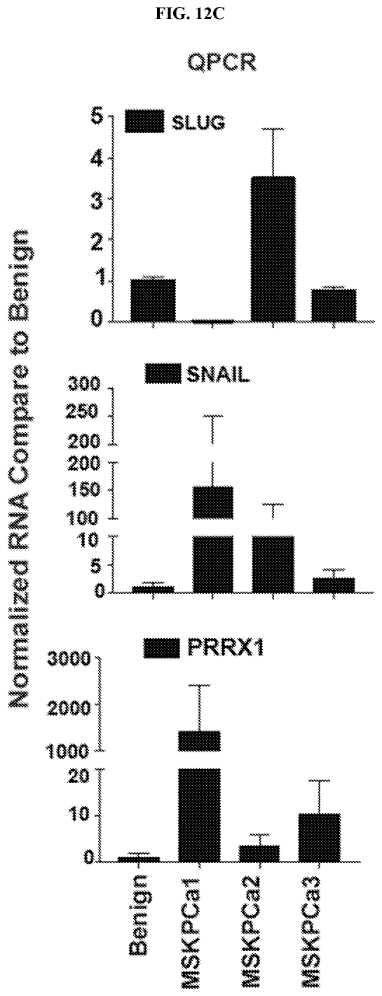

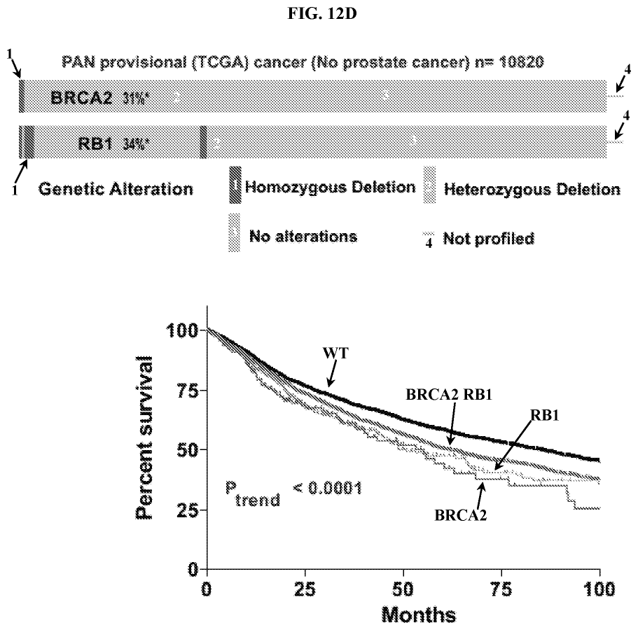

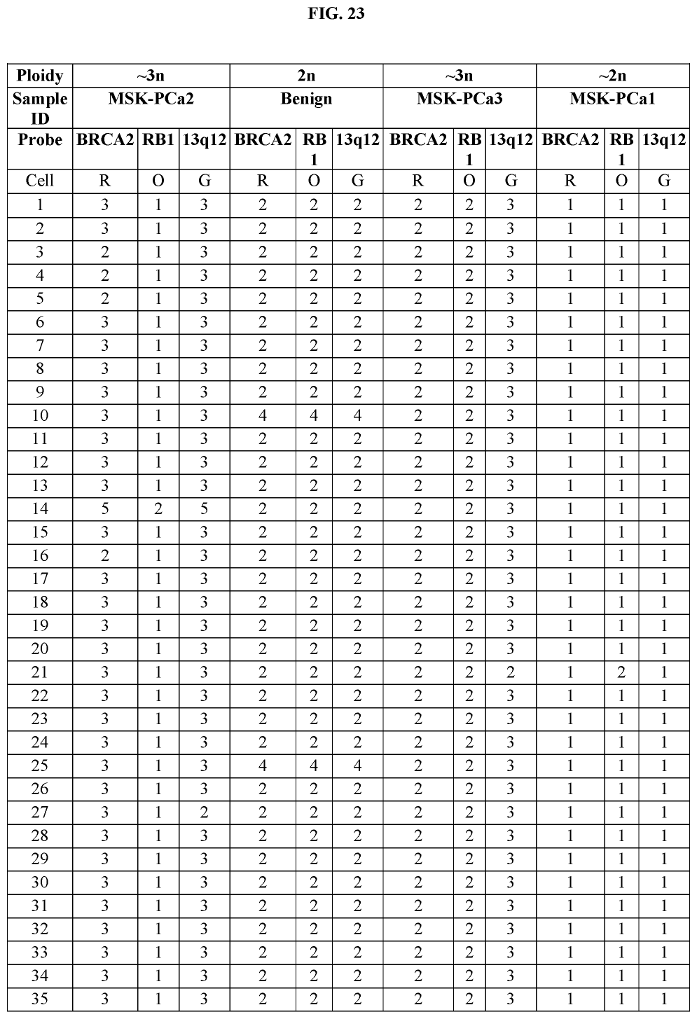

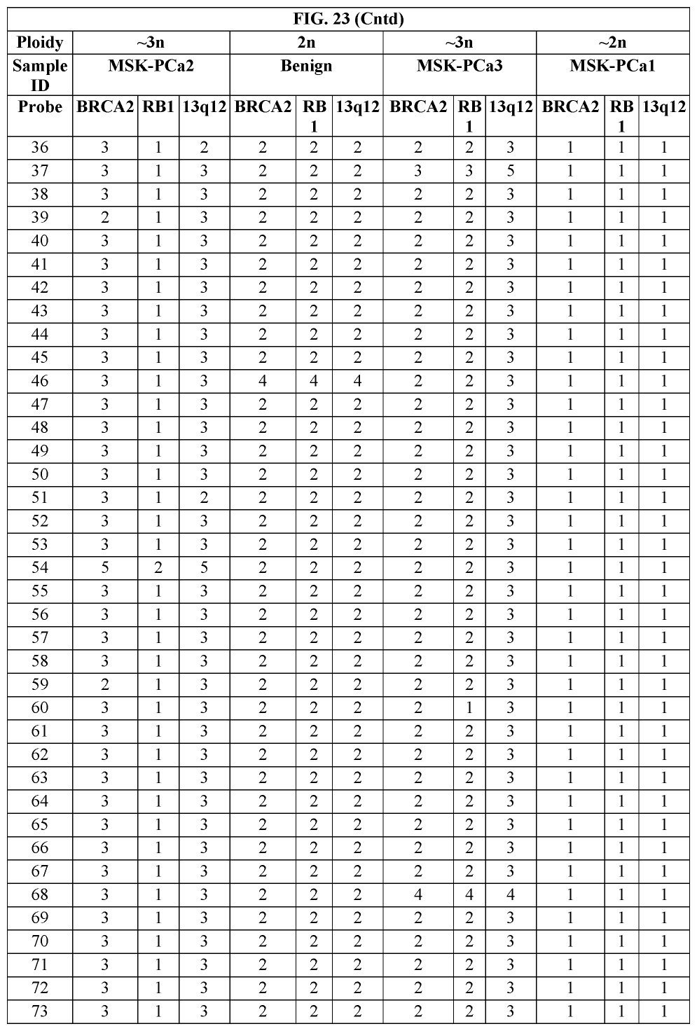

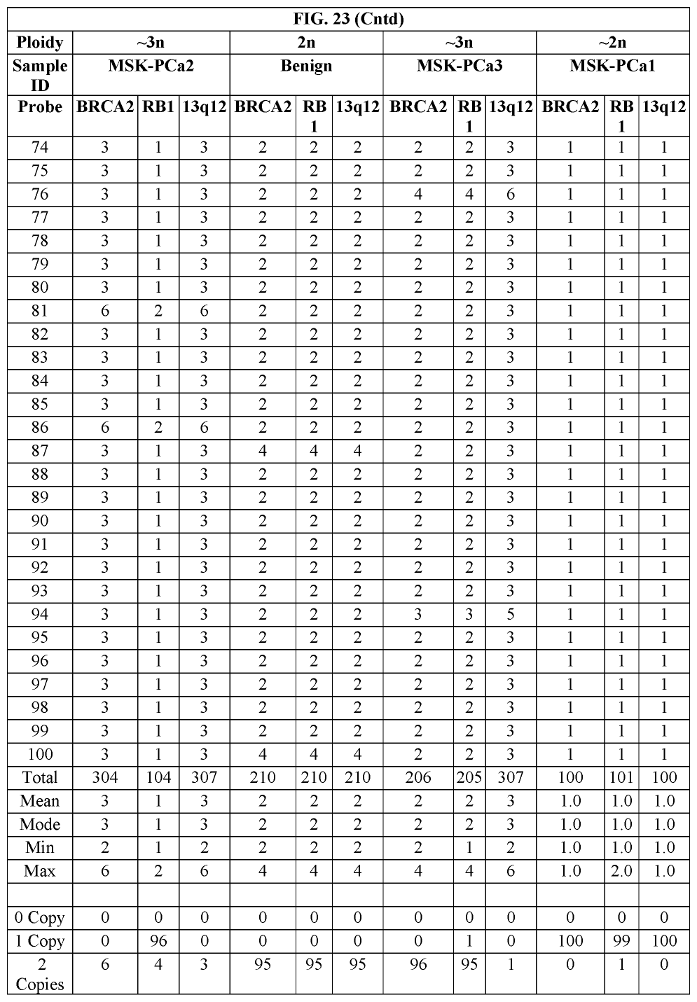

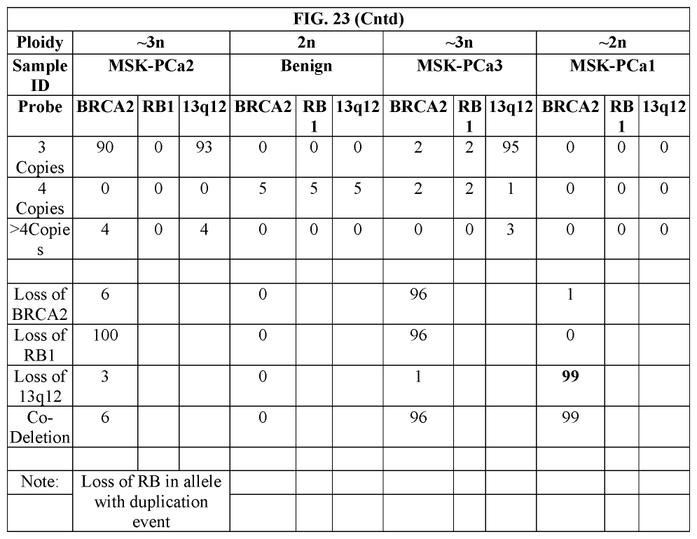

3D organoid cultures of human cancers have shown extreme promise in cancer research. Organoids can potentially be used as avatars for human cancer to study the molecular mechanisms of candidate genes and the effect of drugs. Earlier prostate organoids (MSK-PCa 1-7) were successfully developed from patients with CRPC. These organoids successfully retained the genetic characteristics of patients and grew in vitro as well as in immunodeficient mice. The BRCA2-RB1 status was tested by 3-color FISH in three mCRPC organoids which were originally isolated from metastatic sites from castration-resistant tumors. As a control, a benign prostate organoid was also analyzed by FISH. It was observed that organoid MSK-PCal and MSK-PCa3 exhibited heterozygous co-deletion (˜100% of cells) of BRCA2 and RB1; however, MSK-PCa2 largely (94%) exhibited heterozygous deletion of RB1 only (FIGS. 6A, 6B, 12A, and 23). As shown in FIG. 6C, the copy number segment analysis of the prostate cancer organoids matched the FISH analysis, showing co-deletion of BRCA2 and RB1 in MSK-PCa1 and MSK-PCa3, and deletion of RB1 only in MSK-PCa2. Heterozygous deletion of BRCA2 and RB1 was consistent with loss of their protein expression as identified in the previous observation in TCGA prostate cancer cohort (FIG. 4C). Upregulation of BRCA2 protein expression was observed in the MSK-PCa2 organoid, which may be due to an extra copy of chromosome 13 (FIGS. 12A and 23) rather than due to transcriptional activity of BRCA2. As shown in FIG. 6D, MSK-PCal and MSK-PCa3 also showed upregulation of mesenchymal markers N-cadherin and vimentin (the latter only in MSK-PCal), indicating the EMT-like phenotype of these cells. However, MSK-PCa2 exhibited more epithelial morphology (FIG. 6D). Higher SNAIL and PRRX1 mRNA expression was also observed in the MSK-PCal organoid (FIG. 12C)

As shown in FIG. 6E, growth reduction of MSK-PCal and MSK-PCa3 was observed compared to the benign organoid when treated with the PARPi olaparib and talazoparib. However, PARPi did not have an inhibitory effect on the growth of the MSK-PCa2 organoid. Interestingly, none of the organoids harbored any other known mutation in DDR genes (FIG. 12B), indicating that co-heterozygous deletion of BRCA2 and RB1 is sufficient to sensitize cells to PARPi treatment-mediated growth inhibition.

BRCA2-RB1 deletion (heterozygous and homozygous) was observed in ˜30% of all cancer types determined from TCGA pan-cancer cohort (without prostate cancer n=10,820) (FIG. 12D (top panel) and data not shown). Deletion of either BRCA2 or RB1 or co-deletion was associated with shorter overall survival (ptrend<0.0001) (FIG. 12D, bottom panel), indicating that loss of BRCA2 or RB1 alone may also play an important role in disease progression in the pan-cancer scenario.

These results show that prostate cancer patients harboring a co-deletion in BRCA2- and RB1 are sensitive to treatment with PARP inhibitors. Accordingly, the methods disclosed herein are useful for selecting a prostate cancer patient for treatment with a PARP inhibitor, and/or treating or preventing metastatic castration-resistant prostate cancer in a patient in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any

1. A method for selecting a prostate cancer patient for treatment with a PARP inhibitor comprising:
(a) detecting a co-deletion in BRCA2 and RB1 in a biological sample obtained from a prostate cancer patient; and
(b) administering a PARP inhibitor to the prostate cancer patient, optionally wherein the co-deletion comprises a frameshift mutation or a nonsense mutation in each of BRCA2 and RB1.

2. The method of claim 1, wherein the prostate cancer patient is diagnosed with or at risk for metastatic castration-resistant prostate cancer.

3. The method of claim 1, wherein the co-deletion in BRCA2 and RB1 is homozygous or heterozygous.

4. The method of claim 1, wherein the patient has not previously received an anti-cancer therapy, optionally wherein the anti-cancer therapy is chemotherapy, radiation therapy, surgery or any combination thereof.

5. (canceled)

6. The method of claim 1, wherein the PARP inhibitor is selected from the group consisting of olaparib, rucaparib, niraparib, talazoparib, veliparib, an inhibitory nucleic acid targeting PARP, and an anti-PARP neutralizing antibody, optionally wherein the inhibitory nucleic acid targeting PARP is a shRNA, a siRNA, a sgRNA, a ribozyme, or an anti-sense oligonucleotide.

7. (canceled)

8. The method of claim 1, wherein the prostate cancer is castration-resistant prostate cancer or primary (localized) prostate cancer.

9. The method of claim 1, wherein the co-deletion in BRCA2 and RB1 is detected via polymerase chain reaction (PCR), reverse transcriptase polymerase chain reaction (RT-PCR), next-generation sequencing, Northern blotting, Southern blotting, microarray, dot or slot blots, fluorescent in situ hybridization (FISH), electrophoresis, chromatography, or mass spectroscopy.

10. The method of claim 1, wherein the biological sample is blood, plasma, serum, or a prostate tissue sample.

11. The method of claim 1, wherein the patient harbors a mutation in TP53 and/or ATM.

12. The method of claim 1, wherein the co-deletion results in the production of non-functional BRCA2 and RB1 polypeptides.

13. A method for treating or preventing metastatic castration-resistant prostate cancer in a patient in need thereof comprising administering to the patient an effective amount of a PARP inhibitor, wherein the patient harbors a co-deletion in BRCA2 and RB1, and wherein the co-deletion comprises a frameshift mutation or a nonsense mutation in each of BRCA2 and RB1.

14. The method of claim 13, wherein the co-deletion results in the production of non-functional BRCA2 and RB1 polypeptides.

15. The method of claim 13, wherein the co-deletion in BRCA2 and RB1 is homozygous or heterozygous.

16. The method of claim 13, wherein the patient has not previously received an anti-cancer therapy.

17. The method of claim 16, wherein the anti-cancer therapy is chemotherapy, radiation therapy, surgery or any combination thereof.

18. The method of claim 13, wherein the PARP inhibitor is selected from the group consisting of olaparib, rucaparib, niraparib, talazoparib, veliparib, an inhibitory nucleic acid targeting PARP, and an anti-PARP neutralizing antibody.

19. The method of claim 18, wherein the inhibitory nucleic acid targeting PARP is a shRNA, a siRNA, a sgRNA, a ribozyme, or an anti-sense oligonucleotide.

20. The method of claim 13, wherein the prostate cancer is castration-resistant prostate cancer or primary (localized) prostate cancer.

21. The method of claim 13, wherein the co-deletion in BRCA2 and RB1 in the patient is detected via polymerase chain reaction (PCR), reverse transcriptase polymerase chain reaction (RT-PCR), next-generation sequencing, Northern blotting, Southern blotting, microarray, dot or slot blots, fluorescent in situ hybridization (FISH), electrophoresis, chromatography, or mass spectroscopy.

22. The method of claim 13, wherein the patient harbors a mutation in TP53 and/or ATM.
